(19) United States
(12) Patent Application Publication
(10) Pub. No.: US 2008/0293716 A1
(43) Pub. Date: Nov. 27, 2008

(54) Chemical Compounds

(75) Inventors: David Harold Drewry, Durham, NC (US); Robert Neil Hunter, III, Durham, NC (US); David Kendall Jung, Durham, NC (US); James Andrew Linn, Durham, NC (US); Clark Sehon, King of Prussia, PA (US); Robert A. Stavenger, King of Prussia, PA (US)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(73) Assignee: SMITHKLINE BEECHAM CORPORATION, PHILADELPHIA, PA (US)

(21) Appl. No.: 10/597,475

(22) PCT Filed: Jan. 28, 2005
(86) PCT No.: PCT/US2005/003478

§ 371 Date: Aug. 12, 2008

Related U.S. Application Data
(60) Provisional application No. 60/540,605, filed on Jan. 30, 2004.

(51) Int. Cl. A61K 31/5377; C07D 409/04; A61K 31/4436; C07D 413/14

(52) U.S. Cl. 514/235.5; 546/280.4; 514/336; 544/131

(57) ABSTRACT

There is provided a compound of Formula (I) or a salt, solvate, or physiologically functional derivative thereof:

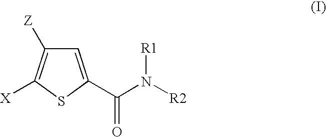

### FIELD OF THE INVENTION

The present invention relates to thiophene amide derivatives, compositions and medicaments containing the same, as well as processes for the preparation and use of such compounds, compositions and medicaments. Such thiophene amide derivatives are potentially useful in the treatment of diseases associated with inappropriate tyrosine and/or serine/threonine kinase activity.

### BACKGROUND OF THE INVENTION

An important large family of enzymes is the protein kinase enzyme family. Currently, there are about 500 different known protein kinases. Protein kinases serve to catalyze the phosphorylation of an amino acid side chain in various proteins by the transfer of the γ-phosphate of the ATP-Mg²⁺ complex to said amino acid side chain. These enzymes control the majority of the signaling processes inside cells, thereby governing cell function, growth, differentiation and destruction (apoptosis) through reversible phosphorylation of the hydroxyl groups of serine, threonine and tyrosine residues in proteins. Studies have shown that protein kinases are key regulators of many cell functions, including signal transduction, transcriptional regulation, cell motility, and cell division. Several oncogenes have also been shown to encode protein kinases, suggesting that kinases play a role in oncogenesis. These processes are highly regulated, often by complex intermeshed pathways where each kinase will itself be regulated by one or more kinases. Consequently, aberrant or inappropriate protein kinase activity can contribute to the rise of disease states associated with such aberrant kinase activity. Due to their physiological relevance, variety and ubiquitousness, protein kinases have become one of the most important and widely studied family of enzymes in biochemical and medical research.

The protein kinase family of enzymes is typically classified into two main subfamilies: Protein Tyrosine Kinases and Protein Serine/Threonine Kinases, based on the amino acid residue they phosphorylate. The serine/threonine kinases (PSTK), includes cyclic AMP- and cyclic GMP-dependent protein kinases, calcium- and phospholipid-dependent protein kinase, calcium- and calmodulin-dependent protein kinases, casein kinases, cell division cycle protein kinases and others. These kinases are usually cytoplasmic or associated with the particulate fractions of cells, possibly by anchoring proteins. Aberrant protein serine/threonine kinase activity has been implicated or is suspected in a number of pathologies such as rheumatoid arthritis, psoriasis, septic shock, bone loss, many cancers and other proliferative diseases. Accordingly, serine/threonine kinases and the signal transduction pathways which they are part of are important targets for drug design. The tyrosine kinases phosphorylate tyrosine residues. Tyrosine kinases play an equally important role in cell regulation. These kinases include several receptors for molecules such as growth factors and hormones, including epidermal growth factor receptor, insulin receptor, platelet derived growth factor receptor and others. Studies have indicated that many tyrosine kinases are transmembrane proteins with their receptor domains located on the outside of the cell and their kinase domains on the inside. Much work is also under progress to identify modulators of tyrosine kinases as well.

A major signal transduction systems utilized by cells is the RhoA-signalling pathways. RhoA is a small GTP binding protein that can be activated by several extracellular stimuli such as growth factor, hormones, mechanic stress, osmotic change as well as high concentration of metabolite like glucose. RhoA activation involves GTP binding, conformation alteration, post-translational modification (geranylgeranyllization and farnesylation) and activation of its intrinsic GTPase activity. Activated RhoA is capable of interacting with several effector proteins including ROCKs and transmit signals into cellular cytoplasm and nucleus.

ROCK1 and 2 constitute a family of kinases that can be activated by RhoA-GTP complex via physical association. Activated ROCKs phosphorylate a number of substrates and play important roles in pivotal cellular functions. The substrates for ROCKs include myosin binding subunit of myosin light chain phosphatase (MBS, also named MYPT1), adducin, moesin, myosin light chain (MLC), LIM kinase as well as transcription factor FHL. The phosphorylation of theses substrates modulate the biological activity of the proteins and thus provide a means to alter cell's response to external stimuli. One well documented example is the participation of ROCK in smooth muscle contraction. Upon stimulation by phenylephrine, smooth muscle from blood vessels contracts. Studies have shown that phenylephrine stimulates alpha-adrenergic receptors and leads to the activation of RhoA. Activated RhoA in turn stimulates kinase activity of ROCK1 and which in turn phosphorylates MBS. Such phosphorylation inhibits the enzyme activity of myosin light chain phosphatase and increases the phosphorylation of myosin light chain itself by a calcium-dependent myosin light chain kinase (MLCK) and consequently increases the contractility of myosin-actin bundle, leading to smooth muscle contraction. This phenomena is also sometimes called calcium sensitization. In addition to smooth muscle contraction, ROCKs have also been shown to be involved in cellular functions including apoptosis, cell migration, transcriptional activation, fibrosis, cytokinesis, inflammation and cell proliferation. Moreover, in neurons ROCK plays a critical role in the inhibition of axonal growth by myelin-associated inhibitory factors such as myelin-associated glycoprotein (MAG). ROCK-activity also mediates the collapse of growth cones in developing neurons. Both processes are thought to be mediated by ROCK-induced phosphorylation of substrates such as LIM kinase and myosin light chain phosphatase, resulting in increased contractility of the neuronal actin-myosin system.

Inhibitors of ROCKs have been suggested for use in the treatments of a variety of diseases. They include cardiovascular diseases such as hypertension, chronic and congestive heart failure, cardiac hypertrophy, restenosis, chronic renal failure and atherosclerosis. In addition, because of its muscle relaxing properties, it is also suitable for asthma, male erectile dysfunctions, female sexual dysfunction and over-active bladder syndrome. ROCK inhibitors have been shown to possess anti-inflammatory properties. Thus they can be used as treatment for neuroinflammatory diseases such as stroke, multiple sclerosis, Alzheimer's disease, Parkinson's disease, amyotrophic lateral sclerosis and inflammatory pain, as well as other inflammatory diseases such as rheumatoid arthritis, irritable bowel syndrome, inflammatory bowel disease. In addition, based on their neurite outgrowth inducing effects, ROCK inhibitors could be useful drugs for neuronal regeneration, inducing new axonal growth and axonal rewiring across lesions within the CNS. ROCK inhibitors are therefore likely to be useful for regenerative (recovery) treatment of CNS disorders such as spinal cord injury, acute neuronal injury (stroke, traumatic brain injury), Parkinsons disease, Alzheimers disease and other neurodegenerative disorders. Since ROCK inhibitors reduce cell proliferation and cell migration, they could be useful in treating cancer and tumor metastasis. Furthermore, there is evidence suggesting that ROCK inhibitors suppress cytoskeletal rearrangement upon virus invasion, thus they also have potential therapeutic value in anti-viral and anti-bacterial applications. ROCK inhibitors may also be useful for the treatment of insulin resistance and diabetes.

The present inventors have discovered novel thiophene amide compounds, which are inhibitors of ROCK activity. Such derivatives are useful in the treatment of disorders associated with inappropriate ROCK activity.

### SUMMARY OF THE INVENTION

In one aspect of the present invention, there is provided a compound of Formula (I) or a salt, solvate, or physiologically functional derivative thereof:

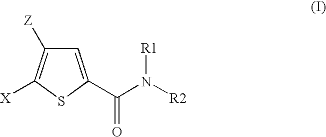

wherein:
R1 is hydrogen or C₁₋₆alkyl;
R2 is selected from the group consisting of C₁₋₆alkyl, C₁₋₄alkylNR⁷R⁸ (wherein R⁷ and R⁸ are independently H or C₁₋₄alkyl), aryl, CH(CH₂OH)aryl, arylC₁₋₆alkyl, aryloxyC₁₋₆alkyl, heteroaryl, heteroarylC₁₋₆alkyl, heterocyclyl and heterocyclylC₁₋₆alkyl, wherein in each case the aryl, heteroaryl or heterocyclyl moiety is optionally substituted by one to five groups selected from the group consisting of halogen, NH₂, hydroxy, cyano, C₁₋₄alkyl, —OCH₂O—, C₁₋₄alkoxy, haloC₁₋₄alkyl, haloC₁₋₄alkoxy, aryl, aryloxy, C₁₋₄alkoxycarbonyl, C₁₋₄hydroxyalkyl, C₁₋₄alkanoyl, C₁₋₄alkylsulfonyl, (CH₂)₀₋₄NHCOOC₁₋₄alkyl, and a group R₃R₄NSO₂ (wherein R₃ and R₄ are independently hydrogen or C₁₋₄alkyl) and a 5- or 6-membered heteroaryl group;
or R1 and R2, together with the nitrogen atom to which they are joined, form a 5- or 6-membered monocyclic heterocyclic ring or a 9- or 10-membered bicyclic heterocyclic ring wherein at least the ring which contains the nitrogen atom to which R1 and R2 are joined is non-aromatic, and wherein the 5- or 6-membered monocyclic heterocyclic ring or the 9or 10-membered bicyclic heterocyclic ring is optionally substituted by one to four groups selected from the group consisting of halogen, hydroxy, cyano, C₁₋₄alkanoyl, oxo, C₁₋₄alkyl, C₁₋₄alkoxy, haloC₁₋₄alkyl, haloC₁₋₄alkoxy, aryl, aryloxy and C₁₋₄alkoxycarbonyl;
X is indazolyl, pyrazolyl or a group

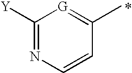

wherein
G is CH or N; and
Y is hydrogen or a group NR5R6 (wherein R5 and R6 are independently hydrogen, C₁₋₆alkyl), (CH₂)₀₋₆-phenyl (wherein the phenyl group is optionally substituted by halogen or OC₁₋₄alkyl);
   and
Z is hydrogen, halogen, cyano or a 5- or 6-membered heteroaryl.

### DETAILED DESCRIPTION OF THE INVENTION

As used herein, the term “C₁₋₄alkyl” refers to a straight or branched alkyl which contains one, two, three or four carbon atoms in all isomeric forms. Examples include methyl, ethyl, propyl, isopropyl, butyl, isobutyl, sec-butyl and tert-butyl. As used herein, the term “C₁₋₆alkyl” refers to a straight or branched alkyl which contains one, two, three, four, five or six carbon atoms in all isomeric forms. Examples include, in addition to those listed above for C₁₋₄alkyl: pentyl, neopentyl, sec-pentyl, n-pentyl, isopentyl, tert-pentyl and hexyl.

As used herein the term C₁₋₄hydroxy alkyl refers to a hydroxy group attached through a C₁₋₄alkylene group.

As used herein, the term “C₁₋₄alkanoyl” refers to an alkanoyl group having from 1 to 4 carbon atoms, such as methanoyl (or “formyl”), ethanoyl (or “acetyl”), propanoyl, isopropanoyl, butanoyl, isobutanoyl and sec-butanoyl.

As used herein, the term “aryl” refers to phenyl or a 8- to 11-membered bicyclic aromatic group wherein Examples include phenyl, indenyl, azulenyl and naphthyl,

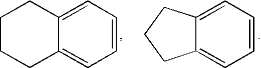

As used herein, the term “arylC₁₋₆alkyl” refers to an aryl group attached through a C₁₋₆alkylene group. The C₁₋₆alkylene group may be in any suitable isomeric form. Examples of arylC₁₋₆alkyl include benzyl, phenethyl (including phenyl-CH₂CH₂— and phenyl-C(CH₃)—) and naphthylmethyl.

As used herein, the term “aryloxy” refers to an aryl group attached via an oxygen atom. Examples of aryloxy include phenyloxy and naphthyloxy.

As used herein, the term “aryloxyC₁₋₆alkyl” refers to an aryloxy group which is attached through a C₁₋₆alkylene group. The C₁₋₆alkylene group may be in any suitable isomeric form. Examples of aryloxyC₁₋₆alkyl include phenoxyethyl.

As used herein, the terms “heteroaryl” and “heteroaromatic group” refer to a 5- or 6-membered monocyclic aromatic group wherein one, two or three carbon atoms are replaced by a heteroatom independently selected from N, O and S, or to a 8- to 11-membered bicyclic aromatic group wherein one to six carbon atoms in total are replaced by a heteroatom independently selected from N, O and S. Examples of 5- or 6-membered heteroaromatic groups include furanyl, thienyl, pyrrolyl, oxazolyl, thiazolyl, imidazolyl, oxadiazolyl, thiadiazolyl, pyridinyl, triazolyl, triazinyl, pyridazyl, pyrimidinyl, isothiazolyl, isoxazolyl, pyrazinyl, pyrazolyl and pyrimidinyl; examples of 8- to 11-membered heteroaromatic groups include indazolyl, quinoxalinyl, quinazolinyl, pyridopyrazinyl, benzoxazolyl, benzothiophenyl, benzimidazolyl, naphthyridinyl, quinolinyl, benzofuranyl, indolyl, benzothiazolyl, pyridopyrimidinyl and isoquinolinyl.

As used herein, the term “heteroarylC₁₋₆alkyl” refers to a heteroaryl group attached through a C₁₋₆alkylene group. The C₁₋₆alkylene group may be in any suitable isomeric form. Examples of arylC₁₋₆alkyl include pyridinylmethyl, pyridinylmethyl (including pyridinyl-CH₂CH₂— and pyridinyl-C(CH₃)—) and benzimidazolylmethyl.

As used herein, the terms “heterocyclyl” refers to a 5- or 6-membered non-aromatic cyclic group containing one, two or three heteroatom(s) independently selected from N, O and S. Examples include pyrrolidinyl, imidazolidinyl, pyrazolidinyl, isothiazolyl, thiazolyl, piperidinyl, piperazinyl, morpholinyl and thiomorpholinyl, tetrahydrofuranyl, dioxolanyl, tetrahydrothienyl, dioxanyl and dithianyl.

As used herein, the term “heterocyclylC₁₋₆alkyl” refers to a heterocyclyl group attached through a C₁₋₆alkylene group. The C₁₋₆alkylene group may be in any suitable isomeric form. Examples of heterocyclylC₁₋₆alkyl include piperidinylmethyl, piperidinylethyl and morpholinylethyl.

As used herein, the term “5- or 6-membered monocyclic heterocyclic ring or a 9- or 10-membered bicyclic heterocyclic ring” refers to a 5- or 6-membered non-aromatic monocyclic heterocyclyl group containing one, two or three heteroatom(s) independently selected from N, O and S, or a 9- or 10-membered bicyclic heterocyclyl group, which contains in total one, two or three heteroatom(s) independently selected from N, O and S, and in which at least one of the rings is non-aromatic. The bicyclic heterocyclic ring may be a fused ring system or a spiro ring system. It should be understood that the 5- or 6-membered monocyclic heterocyclic ring or a 9- or 10-membered bicyclic heterocyclic ring formed by R1 and R2 would be N-linked. Examples of 5- or 6-membered monocyclic heterocyclic rings include pyrrolidinyl, imidazolidinyl, pyrazolidinyl, isothiazolyl, thiazolyl, piperidinyl, piperazinyl, morpholinyl and thiomorpholinyl. Examples of 9- or 10-membered bicyclic heterocyclic rings having a fused structure include tetrahydroisoquinolinyl. Examples of 9- or 10-membered bicyclic heterocyclic rings having a spiro structure include triazaspiro[4.5]decanonyl.

As used herein, the term “halogen” refers to fluorine (F), chlorine (Cl), bromine (Br), or iodine (I) and the term “halo” refers to the halogen radicals: fluoro (—F), chloro (—Cl), bromo (—Br), and iodo (—I).

As used herein, the term “C₁₋₆alkoxy” refers to a straight chain or branched chain alkoxy (or “alkyloxy”) group having from one to six carbon atoms, such as methoxy, ethoxy, propoxy, isopropoxy, butoxy, isobutoxy, sec-butoxy, tert-butoxy, pentoxy, neopentoxy, sec-pentoxy, n-pentoxy, isopentoxy, tert-pentoxy and hexoxy.

As used herein, the term “haloC₁₋₄alkyl” refers to a halogen-substituted C₁₋₄alkyl group such as —CF₃. Similarly, the term “haloC₁₋₄alkoxy” refers to a halogen-substituted C₁₋₄alkoxy group such as CF₃O—.

As used herein, the term “C₁₋₄alkoxycarbonyl” refers to the group (C₁₋₄alkyl)OC(═O)—. Examples of C₁₋₄alkoxycarbonyl include ethyloxycarbonyl (C₂H₅OC(═O)—) and methyloxycarbonyl (CH₃C(═O)—).

As used herein, the term “salt” refers to any salt of a compound according to the present invention prepared from an inorganic or organic acid or base, quaternary ammonium salts and internally formed salts. Physiologically acceptable salts are particularly suitable for medical applications because of their greater aqueous solubility relative to the parent compounds. Such salts must clearly have a physiologically acceptable anion or cation. Suitably physiologically acceptable salts of the compounds of the present invention include acid addition salts formed with inorganic acids such as hydrochloric, hydrobromic, hydroiodic, phosphoric, metaphosphoric, nitric and sulfuric acids, and with organic acids, such as tartaric, acetic, trifluoroacetic, citric, malic, lactic, fumaric, benzoic, formic, propionic, glycolic, gluconic, maleic, succinic, camphorsulfuric, isothionic, mucic, gentisic, isonicotinic, saccharic, glucuronic, furoic, glutamic, ascorbic, anthranilic, salicylic, phenylacetic, mandelic, embonic (pamoic), methanesulfonic, ethanesulfonic, pantothenic, stearic, sulfinilic, alginic, galacturonic and arylsulfonic, for example benzenesulfonic and p-toluenesulfonic, acids; base addition salts formed with alkali metals and alkaline earth metals and organic bases such as N,N-dibenzylethylenediamine, chloroprocaine, choline, diethanolamine, ethylenediamine, meglumaine (N-methylglucamine), lysine and procaine; and internally formed salts. Salts having a non-physiologically acceptable anion or cation are within the scope of the invention as useful intermediates for the preparation of physiologically acceptable salts and/or for use in non-therapeutic, for example, in vitro, situations.

As used herein, the term “solvate” refers to a complex of variable stoichiometry formed by a solute (in this invention, a compound of formula (I) or formula (Ia), or a salt or physiologically functional derivative thereof) and a solvent. Such solvents for the purpose of the invention may not interfere with the biological activity of the solute. Examples of suitable solvents include, but are not limited to, water, methanol, ethanol and acetic acid. Preferably the solvent used is a pharmaceutically acceptable solvent. Examples of suitable pharmaceutically acceptable solvents include water, ethanol and acetic acid. Most preferably the solvent used is water.

As used herein, the term “physiologically functional derivative” refers to any pharmaceutically acceptable derivative of a compound of the present invention, for example, an ester or an amide, which upon administration to a mammal is capable of providing (directly or indirectly) a compound of the present invention or an active metabolite thereof. Such derivatives are clear to those skilled in the art, without undue experimentation, and with reference to the teaching of Burger's Medicinal Chemistry And Drug Discovery, 5^{th} Edition, Vol 1: Principles and Practice, which is incorporated herein by reference to the extent that it teaches physiologically functional derivatives.

As used herein, the term “substituted” refers to substitution with the named substituent or substituents, multiple degrees of substitution being allowed unless otherwise stated.

In one embodiment, R1 is hydrogen.

In one embodiment, R2 is arylC₁₋₆alkyl such as phenylethyl, benzyl or naphthylmethyl, optionally substituted by one or two groups selected from the group consisting of halogen, hydroxy, C₁₋₄alkyl (such as methyl), C₁₋₄alkoxy (such as methoxy or ethoxy), haloC₁₋₄alkyl (such as CF₃), haloC₁₋₄alkoxy (such as CF₃O), thiadiazolyl and a group R₃R₄NSO₂ wherein R₃ and R₄ are independently hydrogen or C₁₋₄alkyl (such as H₂NSO₂— or HCH₃NSO₂—).

In another embodiment, R₂ is phenyl optionally substituted by one or two groups selected from the group consisting of halogen and C₁₋₄alkoxy (such as methoxy or ethoxy).

In another embodiment, R2 is heteroaryl (such as pyridinyl or indazolyl) or heteroarylC₁₋₄alkyl (such as pyridinylmethyl, pyridinylethyl or benzimidazolylmethyl).

In another embodiment, R2 is heterocyclyl (such as piperidinyl or morpholinyl) or heterocyclylC₁₋₆alkyl (such as piperidinylethyl or morpholinylethyl).

In another embodiment, R1 and R2, together with the nitrogen atom to which they are joined, form a 6-membered monocyclic heterocyclic ring (such as piperidinyl or piperazinyl) or a 10-membered bicyclic heterocyclic ring wherein at least the ring which each contains the nitrogen atom to which R1 and R2 are joined is non-aromatic (such as tetrahydroisoquinolinyl or triazaspiro[4.5]decanonyl), wherein the 6-membered monocyclic heterocyclic ring or 10-membered bicyclic heterocyclic ring are both optionally substituted by one or two groups selected from oxo, C₁₋₄alkyl (such as methyl or ethyl), phenyl and C₁₋₄alkoxycarbonyl (such as ethyloxycarbonyl or methyloxycarbonyl).

In one embodiment, X is indazolyl, such as 1-H-indazol-5-yl.

In another embodiment, X is pyrazolyl, such as 1H-pyrazol-4-yl.

In another embodiment, X is 4-pyridinyl and Y is hydrogen, as shown below:

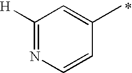

In another embodiment, X is a group:

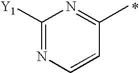

wherein Y₁ is a group NR5R6 whrein R5 and R6 are independently hydrogen or C₁₋₆alkyl, such as —NH₂ or —NHCH₃.

In one embodiment, Z is hydrogen or halogen such as bromine.

In another aspect, the present invention provides a a compound of Formula (Ia) or a salt, solvate, or physiologically functional derivative thereof:

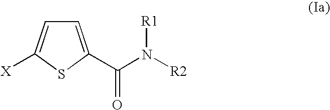

wherein
R1 is hydrogen or C₁₋₆alkyl;
R2 is arylC₁₋₆alkyl optionally substituted by one or two groups selected from the group consisting of halogen, hydroxy, C₁₋₄alkyl, C₁₋₄alkoxy, haloC₁₋₄alkyl, haloC₁₋₄alkoxy, thiadiazolyl and a group R₃R₄NSO₂ wherein R₃ and R₄ are independently hydrogen or C₁₋₄alkyl;
or R1 and R2, together with the nitrogen atom to which they are joined, form a 6-membered monocyclic heterocyclic ring or a 10-membered bicyclic heterocyclic ring wherein the 6-membered monocyclic heterocyclic ring or the 10-membered bicyclic heterocyclic ring are optionally substituted by one or two groups selected from oxo, C₁₋₄alkyl, phenyl and C₁₋₄alkoxycarbonyl;
X is indazolyl, pyrazolyl, 4-pyridinyl or a group

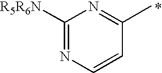

wherein R₅ and R₆ are independently hydrogen or methyl.

In one embodiment of formula (Ia), R₂ is phenylethyl, benzyl or naphthylmethyl, optionally substituted by one or two groups selected from the group consisting of halogen, hydroxy, C₁₋₄alkyl (such as methyl), C₁₋₄alkoxy (such as methoxy or ethoxy), haloC₁₋₄alkyl (such as —CF₃), haloC₁₋₄alkoxy (such as CF₃O—), thiadiazolyl and a group R₃R₄NSO₂ wherein R₃ and R₄ are independently hydrogen or C₁₋₄alkyl (such as H₂NSO₂— or HCH₃NSO₂—).

In another embodiment of formula (Ia), R1 and R2, together with the nitrogen atom to which they are joined, form piperidinyl, piperazinyl, tetrahydroisoquinolinyl or triazaspiro[4.5]decanonyl, wherein the 6-membered monocyclic heterocyclic ring or 10-membered bicyclic heterocyclic ring are both optionally substituted by one or two groups selected from oxo, C₁₋₄alkyl (such as methyl or ethyl), phenyl and C₁₋₄alkoxycarbonyl (such as ethyloxycarbonyl or methyloxycarbonyl).

In one embodiment of formula (Ia), X is 1-H-indazol-5-yl, 1H-pyrazol-4-yl, 4-pyridinyl, 2-amino-4-pyrimidinyl or 2-methylamino-4-pyrimidinyl.

Specific examples of compounds of the present invention include:
N-(2-phenylethyl)-5-(4-pyridinyl)-2-thiophenecarboxamide
N-(3-methoxybenzyl)-5-(4-pyridinyl)-2-thiophenecarboxamide
5-(4-pyridinyl)-N-(2-pyridinylmethyl)-2-thiophenecarboxamide
N-(1-naphthylmethyl)-5-(4-pyridinyl)-2-thiophenecarboxamide
N-(2-ethoxybenzyl)-5-(4-pyridinyl)-2-thiophenecarboxamide
N-(2-bromobenzyl)-5-(4-pyridinyl)-2-thiophenecarboxamide
N-(2-fluorobenzyl)-5-(4-pyridinyl)-2-thiophenecarboxamide
N-(2-chlorobenzyl)-5-(4-pyridinyl)-2-thiophenecarboxamide
N-(2-methylbenzyl)-5-(4-pyridinyl)-2-thiophenecarboxamide
N-(2-trifluoromethylbenzyl)-5-(4-pyridinyl)-2-thiophenecarboxamide
N-(2-trifluoromethoxybenzyl)-5-(4-pyridinyl)-2-thiophenecarboxamide
N-(3-trifluoromethylbenzyl)-5-(4-pyridinyl)-2-thiophenecarboxamide
N-(3-fluorobenzyl)-5-(4-pyridinyl)-2-thiophenecarboxamide
N-(3-chlorobenzyl)-5-(4-pyridinyl)-2-thiophenecarboxamide
N-(3-bromobenzyl)-5-(4-pyridinyl)-2-thiophenecarboxamide
N-(3-iodobenzyl)-5-(4-pyridinyl)-2-thiophenecarboxamide
N-(3-methylbenzyl)-5-(4-pyridinyl)-2-thiophenecarboxamide
N-(3-methoxybenzyl)-5-(4-pyridinyl)-2-thiophenecarboxamide
N-(3-trifluoromethoxybenzyl)-5-(4-pyridinyl)-2-thiophenecarboxamide
N-(3-phenoxybenzyl)-5-(4-pyridinyl)-2-thiophenecarboxamide
N-(4-fluorobenzyl)-5-(4-pyridinyl)-2-thiophenecarboxamide
N-(4-bromobenzyl)-5-(4-pyridinyl)-2-thiophenecarboxamide
N-(4-iodobenzyl)-5-(4-pyridinyl)-2-thiophenecarboxamide
N-(4-trifluoromethylbenzyl)-5-(4-pyridinyl)-2-thiophenecarboxamide
N-(4-methylbenzyl)-5-(4-pyridinyl)-2-thiophenecarboxamide
N-(4-trifluoromethoxybenzyl)-5-(4-pyridinyl)-2-thiophenecarboxamide
N-[4-(aminosulfonyl)benzyl]-5-(4-pyridinyl)-2-thiophenecarboxamide
N-[4-(methylsulfonyl)benzyl]-5-(4-pyridinyl)-2-thiophenecarboxamide
5-(4-pyridinyl)-N-(4-pyridinylmethyl)-2-thiophenecarboxamide
N-benzyl-N-methyl-5-(4-pyridinyl)-2-thiophenecarboxamide
5-(4-pyridinyl)-N-[4-(1,2,3-thiadiazol-4-yl)benzyl]-2-thiophenecarboxamide
5-(4-pyridinyl)-N-(3-pyridinylmethyl)-2-thiophenecarboxamide
N-[2-(2-methylphenyl)ethyl]-5-(4-pyridinyl)-2-thiophenecarboxamide
N-[2-(3-methylphenyl)ethyl]-5-(4-pyridinyl)-2-thiophenecarboxamide
N-[2-(4-methylphenyl)ethyl]-5-(4-pyridinyl)-2-thiophenecarboxamide
N-[2-(2-fluorophenyl)ethyl]-5-(4-pyridinyl)-2-thiophenecarboxamide
N-[2-(3-fluorophenyl)ethyl]-5-(4-pyridinyl)-2-thiophenecarboxamide
N-[2-(4-fluorophenyl)ethyl]-5-(4-pyridinyl)-2-thiophenecarboxamide
N-[2-(2-methoxyphenyl)ethyl]-5-(4-pyridinyl)-2-thiophenecarboxamide
N-[2-(3-methoxyphenyl)ethyl]-5-(4-pyridinyl)-2-thiophenecarboxamide
N-[2-(4-methoxyphenyl)ethyl]-5-(4-pyridinyl)-2-thiophenecarboxamide
N-[2-(2-chlorophenyl)ethyl]-5-(4-pyridinyl)-2-thiophenecarboxamide
N-[2-(3-chlorophenyl)ethyl]-5-(4-pyridinyl)-2-thiophenecarboxamide
N-[2-(4-chlorophenyl)ethyl]-5-(4-pyridinyl)-2-thiophenecarboxamide
N-[2-(2-ethoxyphenyl)ethyl]-5-(4-pyridinyl)-2-thiophenecarboxamide
N-[2-(3-ethoxyphenyl)ethyl]-5-(4-pyridinyl)-2-thiophenecarboxamide
N-[2-(4-ethoxyphenyl)ethyl]-5-(4-pyridinyl)-2-thiophenecarboxamide
N-[2-(2-bromophenyl)ethyl]-5-(4-pyridinyl)-2-thiophenecarboxamide
N-[2-(3-bromophenyl)ethyl]-5-(4-pyridinyl)-2-thiophenecarboxamide
N-[2-(4-bromophenyl)ethyl]-5-(4-pyridinyl)-2-thiophenecarboxamide
N-[2-(2-phenoxyphenyl)ethyl]-5-(4-pyridinyl)-2-thiophenecarboxamide
N-[2-(4-phenoxyphenyl)ethyl]-5-(4-pyridinyl)-2-thiophenecarboxamide
N-[2-(4-hydroxyphenyl)ethyl]-5-(4-pyridinyl)-2-thiophenecarboxamide
N-[2-(3-trifluoromethylphenyl)ethyl]-5-(4-pyridinyl)-2-thiophenecarboxamide
N-{2-[4-(aminosulfonyl)phenyl]ethyl}-5-(4-pyridinyl)-2-thiophenecarboxamide
2-{[5-(4-pyridinyl)-2-thienyl]carbonyl}-1,2,3,4-tetrahydroisoquinoline
5-(4-pyridinyl)-N-[2-(3-pyridinyl)ethyl]-2-thiophenecarboxamide
5-(4-pyridinyl)-N-[2-(4-pyridinyl)ethyl]-2-thiophenecarboxamide
N-(2-phenoxyethyl)-5-(4-pyridinyl)-2-thiophenecarboxamide
N-[2-(1-piperidinyl)ethyl]-5-(4-pyridinyl)-2-thiophenecarboxamide
N-[2-(4-morpholinyl)ethyl]-5-(4-pyridinyl)-2-thiophenecarboxamide
1-phenyl-4-{[5-(4-pyridinyl)-2-thienyl]carbonyl}piperazine
N-(1H-indazol-5-yl)-5-(4-pyridinyl)-2-thiophenecarboxamide
1-phenyl-8-{[5-(4-pyridinyl)-2-thienyl]carbonyl}-1,3,8-triazaspiro[4.5]decan-4-one
ethyl 4-({[5-(4-pyridinyl)-2-thienyl]carbonyl}amino)-1-piperidinecarboxylate
ethyl 1-{[5-(4-pyridinyl)-2-thienyl]carbonyl}-4-piperidinecarboxylate
N-(1H-benzimidazol-2-ylmethyl)-5-(4-pyridinyl)-2-thiophenecarboxamide
5-(4-pyridinyl)-N-[2-(2-pyridinyl)ethyl]-2-thiophenecarboxamide
N-[2-(3-hydroxyphenyl)ethyl]-5-(4-pyridinyl)-2-thiophenecarboxamide
N-[(1R)-1-phenylethyl]-5-(4-pyridinyl)-2-thiophenecarboxamide
N-[(1S)-1-phenylethyl]-5-(4-pyridinyl)-2-thiophenecarboxamide
N-[(1R)-1-(3-methoxyphenyl)ethyl]-5-(4-pyridinyl)-2-thiophenecarboxamide
N-[(1S)-1-(3-methoxyphenyl)ethyl]-5-(4-pyridinyl)-2-thiophenecarboxamide
N-isopropyl-5-(4-pyridinyl)-2-thiophenecarboxamide
1-methyl-4-{[5-(4-pyridinyl)-2-thienyl]carbonyl}piperazine
N-phenyl-5-(4-pyridinyl)-2-thiophenecarboxamide
N-(2-methoxyphenyl)-5-(4-pyridinyl)-2-thiophenecarboxamide
N-(2-chlorophenyl)-5-(4-pyridinyl)-2-thiophenecarboxamide
N-(4-methoxyphenyl)-5-(4-pyridinyl)-2-thiophenecarboxamide
N-(4-chlorophenyl)-5-(4-pyridinyl)-2-thiophenecarboxamide
N-benzyl-5-(4-pyridinyl)-2-thiophenecarboxamide
5-(2-amino-4-pyrimidinyl)-N-(3-methoxybenzyl)-2-thiophenecarboxamide
5-(2-amino-4-pyrimidinyl)-N-benzyl-2-thiophenecarboxamide
5-(4-pyrimidinyl)-N-(3-methoxybenzyl)-2-thiophenecarboxamide
5-(1H-indazol-5-yl)-N-(3-methoxybenzyl)-2-thiophenecarboxamide
5-(6-amino-4-pyrimidinyl)-N-(3-methoxybenzyl)-2-thiophenecarboxamide
N-benzyl-4-bromo-5-(4-pyridinyl)-2-thiophenecarboxamide
N-benzyl-4,5-di(4-pyridinyl)-2-thiophenecarboxamide
N-(3-methoxybenzyl)-4,5-di(4-pyridinyl)-2-thiophenecarboxamide
N-benzyl-5-[2-(methylamino)-4-pyrimidinyl]-2-thiophenecarboxamide
N-benzyl-5-(1H-pyrazol-4-yl)-2-thiophenecarboxamide
N-(3-methoxybenzyl)-5-(1H-pyrazol-4-yl)-2-thiophenecarboxamide
5-(2-amino-4-pyrimidinyl)-N-[3-(dimethylamino)-2,2-dimethylpropyl]-2-thiophenecarboxamide
5-(2-amino-4-pyrimidinyl)-N-(2-phenylethyl)-2-thiophenecarboxamide
5-(2-amino-4-pyrimidinyl)-N-(phenylmethyl)-2-thiophenecarboxamide
5-{2-[(4-fluorophenyl)amino]-4-pyrimidinyl}-N-(phenylmethyl)-2-thiophenecarboxamide
5-{2-[(4-chlorophenyl)amino]-4-pyrimidinyl}-N-(phenylmethyl)-2-thiophenecarboxamide
5-{2-[4-(methyloxy)phenyl]amino}-4-pyrimidinyl)-N-(phenylmethyl)-2-thiophenecarboxamide
5-[2-(methylamino)-4-pyrimidinyl]-N-(phenyl methyl)-2-thiophenecarboxamide
N-(phenylmethyl)-5-{2-[(phenylmethyl)amino]-4-pyrimidinyl}-2-thiophenecarboxamide
5-(2-amino-4-pyrimidinyl)-N-{[3-(methyloxy)phenyl]methyl}-2-thiophenecarboxamide
5-(2-{[4-(methyloxy)phenyl]amino}-4-pyrimidinyl)-N{[3-(methyloxy)phenyl]methyl}-2-thiophenecarboxamide
N-[(2-fluorophenyl)methyl]-5-(2-{[4-(methyloxy)phenyl]amino}-4-pyrimidinyl)-2-thiophenecarboxamide
N-[(3-fluorophenyl)methyl]-5-(2-{[4-(methyloxy)phenyl]amino}-4-pyrimidinyl)-2-thiophenecarboxamide
N-[(4-fluorophenyl)methyl]-5-(2-{[4-(methyloxy)phenyl]amino}-4-pyrimidinyl)-2-thiophenecarboxamide
N-[(2-chlorophenyl)methyl]-5-(2-{[4-(methyloxy)phenyl]amino}-4-pyrimidinyl)-2-thiophenecarboxamide
N-[(3-chlorophenyl)methyl]-5-(2-{[4-(methyloxy)phenyl]amino}-4-pyrimidinyl)-2-3 thiophenecarboxamide
N-[(4-chlorophenyl)methyl]-5-(2-{[4-(methyloxy)phenyl]amino}-4-pyrimidinyl)-2-thiophenecarboxamide
5-(2-{[4-(methyloxy)phenyl]amino}-4-pyrimidinyl)-N-{[2-(methyloxy)phenyl]methyl}-2-thiophenecarboxamide
5-(2-[4-(methyloxy)phenyl]amino)-4-pyrimidinyl)-N-{[4-(methyloxy)phenyl]methyl}-2-thiophenecarboxamide
N-(2,3-dihydro-1H-inden-1-yl)-5-(2-{[4-(methyloxy)phenyl]amino}-4-pyrimidinyl)-2-thiophenecarboxamide
5-(2-{[4-(methyloxy)phenyl]amino}-4-pyrimidinyl)-N-(2-phenylethyl)-2-thiophenecarboxamide
D 5-(2-{[4-(methyloxy)phenyl]amino}-4-pyrimidinyl)-N-[(1R)-1-phenylethyl]-2-thiophenecarboxamide
5-(2-{[4-(methyloxy)phenyl]amino}-4-pyrimidinyl)-N-[(1S)-1-phenylethyl]-2-thiophenecarboxamide
5-(2-amino-4-pyrimidinyl)-N-[(2-fluorophenyl)methyl]-2-thiophenecarboxamide
5-(2-amino-4-pyrimidinyl)-N-{[2-(methyloxy)phenyl]methyl}-2-thiophenecarboxamide
5-(2-amino-4-pyrimidinyl)-N-[(2-methylphenyl)methyl]-2-thiophenecarboxamide
5-(2-amino-4-pyrimidinyl)-N-{[4-(methyloxy)phenyl]methyl}-2-thiophenecarboxamide
5-(2-amino-4-pyrimidinyl)-N-[(3-chlorophenyl)methyl]-2-thiophenecarboxamide
5-(2-amino-4-pyrimidinyl)-N-[(2-chlorophenyl)methyl]-2-thiophenecarboxamide
5-(2-amino-4-pyrimidinyl)-N-{(1S)-1-[3-(methyloxy)phenyl]ethyl}-2-thiophenecarboxamide
5-(2-amino-4-pyrimidinyl)-N-{(1R)-1-[3-(methyloxy)phenyl]ethyl}-2-thiophenecarboxamide
5-(2-amino-4-pyrimidinyl)-N-[(4-chlorophenyl)methyl]-2-thiophenecarboxamide
5-(2-amino-4-pyrimidinyl)-N-[(4-fluorophenyl)methyl]-2-thiophenecarboxamide
1,1-dimethylethyl ({4-[({[5-(2-amino-4-pyrimidinyl)-2-thienyl]carbonyl}amino)methyl]phenyl}methyl)carbamate
5-(2-amino-4-pyrimidinyl)-N-{1-[3-(methyloxy)phenyl]ethyl}-2-thiophenecarboxamide
5-(2-amino-4-pyrimidinyl)-N-(1-phenylpropyl)-2-thiophenecarboxamide
5-(2-amino-4-pyrimidinyl)-N-phenyl-2-thiophenecarboxamide
5-(2-amino-4-pyrimidinyl)-N-[(3-bromophenyl)methyl]-2-thiophenecarboxamide
5-(2-amino-4-pyrimidinyl)-N-[(1S)-1-phenylpropyl]-2-thiophenecarboxamide
5-(2-amino-4-pyrimidinyl)-N-[(1R)-1-phenylethyl]-2-thiophenecarboxamide
5-(2-amino-4-pyrimidinyl)-N-[(3-fluorophenyl)methyl]-2-thiophenecarboxamide
5-(2-amino-4-pyrimidinyl)-N-({3-[(difluoromethyl)oxy]phenyl}methyl)-2-thiophenecarboxamide
5-(2-amino-4-pyrimidinyl)-N-(1-methyl-1-phenylethyl)-2-thiophenecarboxamide
N-[(3-aminophenyl)methyl]-5-(2-amino-4-pyrimidinyl)-2-thiophenecarboxamide
5-(2-amino-4-pyrimidinyl)-N-(2,3-dihydro-1H-inden-1-yl)-2-thiophenecarboxamide
5-(2-amino-4-pyrimidinyl)-N-[(1S)-1,2,3,4-tetrahydro-1-naphthalenyl]-2-thiophenecarboxamide
5-(2-amino-4-pyrimidinyl)-N-[(1R)-1,2,3,4-tetrahydro-1-naphthalenyl]-2-thiophenecarboxamide
1,1-dimethylethyl {3-[1-({[5-(2-amino-4-pyrimidinyl)-2-thienyl]carbonyl}amino)ethyl]phenyl}carbamate
5-(2-amino-4-pyrimidinyl)-N-[(2,4-dichlorophenyl)methyl]-2-thiophenecarboxamide
5-(2-amino-4-pyrimidinyl)-N-[(1S)-1-phenylethyl]-2-thiophenecarboxamide
5-(2-amino-4-pyrimidinyl)-N-{[3-(trifluoromethyl)phenyl]methyl}-2-thiophenecarboxamide
5-(2-amino-4-pyrimidinyl)-N-(3-biphenylylmethyl)-2-thiophenecarboxamide
4-[5-(2,3-dihydro-1H-indol-1-ylcarbonyl)-2-thienyl]-2-pyrimidinamine
4-[5-(3,4-dihydro-1(2H)-quinolinylcarbonyl)-2-thienyl]-2-pyrimidinamine
5-(2-amino-4-pyrimidinyl)-N-[(3-hydroxyphenyl)methyl]-2-thiophenecarboxamide
4-[5-(3,4-dihydro-2(1H)-isoquinolinylcarbonyl)-2-thienyl]-2-pyrimidinamine
5-(2-amino-4-pyrimidinyl)-N-(1,3-benzodioxol-5-ylmethyl)-2-thiophenecarboxamide
5-(2-amino-4-pyrimidinyl)-N-[(1R)-1-(3-hydroxyphenyl)ethyl]-2-thiophenecarboxamide
5-(2-amino-4-pyrimidinyl)-N-[(1S,2R)-2-hydroxy-2,3-dihydro-1H-inden-1-yl]-2-thiophenecarboxamide
5-(2-amino-4-pyrimidinyl)-N-[(1S)-2-hydroxy-1-phenylethyl]-2-thiophenecarboxamide
5-(2-amino-4-pyrimidinyl)-N-[(1R,2S)-2-hydroxy-2,3-dihydro-1H-inden-1-yl]-2-thiophenecarboxamide
5-(2-amino-4-pyrimidinyl)-N-[1-(3,5-dichlorophenyl)-2-hydroxyethyl]-2-thiophenecarboxamide
5-(2-amino-4-pyrimidinyl)-N-[(1R)-2-hydroxy-1-phenylethyl]-2-thiophenecarboxamide
   and their salts, solvates and physiologically functional derivatives thereof.

The compounds of formulae (I) and (Ia) have the ability to crystallise in more than one form, a characteristic, which is known as polymorphism, and it is understood that such polymorphic forms (“polymorphs”) are within the scope of formulae (I) and (Ia). Polymorphism generally can occur as a response to changes in temperature or pressure or both and can also result from variations in the crystallisation process. Polymorphs can be distinguished by various physical characteristics known in the art such as x-ray diffraction patterns, solubility, and melting point.

Certain of the compounds described herein may exist in stereoisomeric forms (i.e. they may contain one or more asymmetric carbon atoms or may exhibit cis-trans isomerism). The individual stereoisomers (enantiomers and diastereoisomers) and mixtures of these are included within the scope of the present invention. Likewise, it is understood that compounds of formulae (I) and (Ia) may exist in tautomeric forms other than that shown in the formulae and these are also included within the scope of the present invention.

As referred to above, individual enantiomers of compounds of formulae (I) and (Ia) may be prepared and an indication of the preferred stereochemistry for such enantiomers has been given. In a preferred embodiment, an optically pure enantiomer is desired. The term “optically pure enantiomer” means that the compound contains greater than about 90% of the desired isomer by weight, preferably greater than about 95% of the desired isomer by weight, and most preferably greater than about 99% of the desired isomer by weight, said weight percent based upon the total weight of the isomer(s) of the compound.

It is to be understood that the following embodiments refer to compounds within the scope of both formula (I) and formula (Ia) as defined above unless specifically limited by the definition of each formula or specifically limited otherwise. It is also understood that the embodiments of the present invention described herein, including uses and compositions, are applicable to both formula (I) and formula (Ia).

The compounds of this invention may be made by a variety of methods, including standard chemistry. Any previously defined variable will continue to have the previously defined meaning unless otherwise indicated. Illustrative general synthetic methods are set out below and then specific compounds of the invention are prepared in the Working Examples.

Compounds of general formula (I) may be prepared by methods known in the art of organic synthesis as set forth in part by the following synthesis schemes. In all of the schemes described below, it is well understood that protecting groups for sensitive or reactive groups are employed where necessary in accordance with general principles of chemistry. Protecting groups are manipulated according to standard methods of organic synthesis (T. W. Green and P. G. M. Wuts (1991) Protecting Groups in Organic Synthesis, John Wiley & Sons). These groups are removed at a convenient stage of the compound synthesis using methods that are readily apparent to those skilled in the art. The selection of processes as well as the reaction conditions and order of their execution shall be consistent with the preparation of compounds of Formula (I).

The compounds of formula (I) and (Ia) may be made by a variety of methods, including standard chemistry. Any previously defined variable will continue to have the previously defined meaning unless otherwise indicated. Illustrative general synthetic methods are in Schemes 1, 2 3 and 4. While the Schemes illustrate cases wherein Z is hydrogen, it should be noted that they are also applicable for cases where Z is not hydrogen.

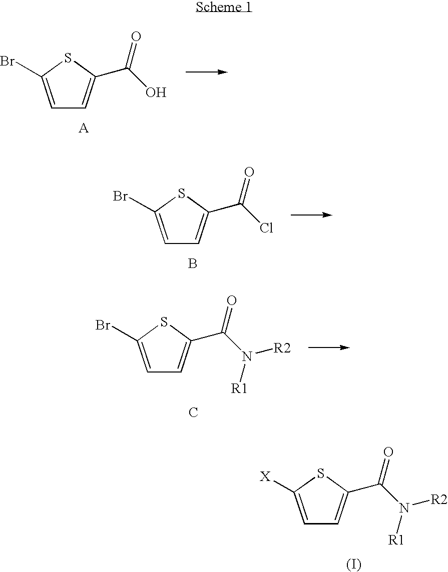

As illustrated in Scheme 1, compounds of general formula (I) may be synthesized starting with compound A, 5-bromo-2-thiophenecarboxylic acid. Compound A can be converted to the acid chloride using an appropriate chlorinating reagent, such as oxalyl chloride or thionyl chloride, to give B. Intermediate B can be reacted with an amine in an appropriate solvent at temperatures between 25 and 250° C., often in the presence of an appropriate additive. For example, reaction of B with phenethylamine in tetrahydrofuran with triethylamine as acid scavenger for 18 hours provides compound C. Using Suzuki reaction conditions, compound C can be reacted with appropriate heteroaryl boronic acids to give compounds of formula (I).

Compounds of general formula (I) can be synthesized from compounds of general formula C through a variety of metal mediated coupling reactions well known to those skilled in the art. For example, reaction of aryl halides such as C with an aryl tin species or an aryl boronic acid species can be carried out in an appropriate solvent in the presence of an appropriate catalyst and an appropriate base at a temperature between 30° C. and 250° C. These reactions (Suzuki reaction with an aryl boronic acid and Stille reaction with an aryl tin reagent) are well described in the literature, and a number of catalyst, base, solvent, and temperature combinations have proven useful. For example, heating an appropriate compound of general formula C with an aryl boronic acid, aqueous sodium carbonate and dichlorobis(triphenylphosphine) palladium (II) in dimethoxyethane at 150° C. for 10 minutes in a SmithSynthesizer microwave is one method useful for synthesis of products of general formula (I). Other well described reactions such as the Heck reaction, Sonogashira reaction, carbonylation reactions and cyanation reactions may be used to generate other compounds of general formula (I) that replace the bromine of compounds C with different functionality, such as substituted olefins, substituted acetylenes, substituted amides, a carboxylic acid, or nitrile. Like the Suzuki and Stille reactions, a number of catalyst, base, solvent, and temperature combinations have proven useful to carry out the Sonogashira reactions, Heck reactions, carbonylation reactions, and cyanations.

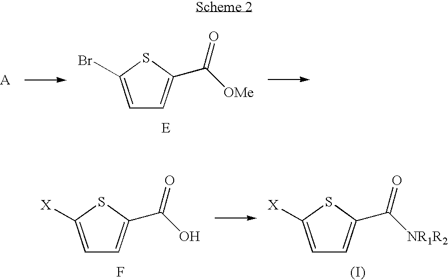

Compounds of general formula (I) may also be synthesized as depicted in Scheme 2. Compound of formula E may be synthesized by reaction of compound A, 5-bromo-2-thiophenecarboxylic acid, with a suitable alkylating agent in a suitable solvent. There are a variety of conditions known in the chemical literature that are useful for alkylating a heteroaromatic carboxylic acid. For example, one can utilize methyl iodide in dimethylformamide with potassium carbonate present. Compounds of general formula F can be synthesized by a variety of metal mediated coupling reactions that are well-described in the literature and known to one skilled in the art. These include, but are not limited to, Heck reactions, Suzuki reactions, Stille reactions, Sonogashira reactions, carbonylation reactions, and cyanation reactions. For all of these types of reactions, a number of catalyst, base, solvent, and temperature combinations have been explored and have proven useful for carrying out the desired transformation. For example, reaction of aryl halides such as E with an aryl boronic acid species can be carried out in an appropriate solvent in the presence of an appropriate catalyst and an appropriate base at a temperature between 30° C. and 250° C. These Suzuki reactions are well described in the literature, and a number of catalyst, base, solvent, and temperature combinations have proven useful. For example, heating an appropriate compound of general formula E with an aryl boronic acid, aqueous sodium carbonate and dichlorobis(triphenylphosphine) palladium (II) in a mixture of dimethoxyethane:ethanol (2:1) at 175° C. for 10 minutes in a SmithSynthesizer™ microwave instrument is one method useful for synthesis of products of general formula F. Where the ester group a compound of formula E is intact after conversion of a compound of formula E to a compound of formula F, the ester group will need to be hydrolysed subsequently using standard conditions known to the person skilled in the art.

Compounds of general formula (I) may be synthesized from compounds of general formula F by activation of the carboxylic acid with a suitable carbodiimide reagent in an appropriate solvent at temperatures between 30 and 250° C., followed by addition of the amine to form an amide bond. For example, reaction of a compound of general formula F, EDC hydrochloride, HOBt and phenethylamine in DMF as solvent at ambient temperature for 18 hours affords compounds of general formula (I).

Compounds of general formula (I) can be synthesized from compounds of general formula E through a variety of metal mediated coupling reactions well known to those skilled in the art. For example, reaction of aryl halides such as E with an aryl tin species or an aryl boronic acid species can be carried out in an appropriate solvent in the presence of an appropriate catalyst and an appropriate base at a temperature between 30° C. and 250° C. These reactions (Suzuki reaction with an aryl boronic acid and Stille reaction with an aryl tin reagent) are well described in the literature, and a number of catalyst, base, solvent, and temperature combinations have proven useful. For example, heating an appropriate compound of general formula E with an aryl boronic acid, aqueous sodium carbonate and dichlorobis(triphenylphosphine) palladium (II) in dimethoxyethane at 150° C. for 10 minutes in a SmithSynthesizer microwave is one method useful for synthesis of products of general formula (I). Other well described reactions such as the Heck reaction, Sonogashira reaction, carbonylation reactions and cyanation reactions may be used to generate other compounds of general formula (I) that replace the bromine of compounds E with different functionality, such as substituted olefins, substituted acetylenes, substituted amides, a carboxylic acid, or nitrile. Like the Suzuki and Stille reactions, a number of catalyst, base, solvent, and temperature combinations have proven useful to carry out the Sonogashira reactions, Heck reactions, carbonylation reactions, and cyanations.

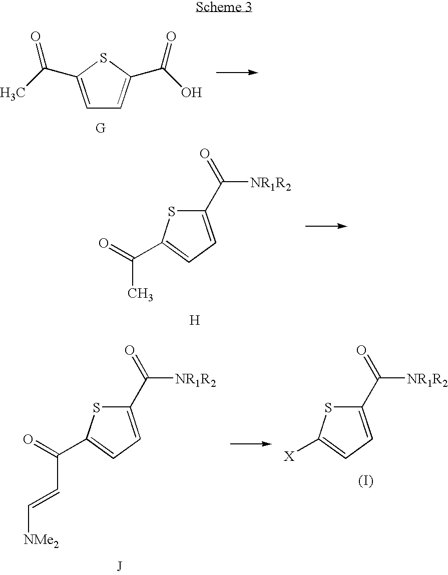

### Scheme 3

Scheme 3 depicts an alternate way to synthesize compounds of general formula (I). Compound H can be synthesized by reaction of compound G using a suitable amide coupling reaction in a suitable solvent at a suitable temperature. There are a variety of conditions known in the chemical literature that are useful for amide coupling reactions of a heteroaromatic carboxylic acid with an amine as outlined in Scheme 2 above. Compounds of formula J can be prepared from compounds of formula H by heating with dimethylformamide dimethylacetal. Application of these sorts of conditions, as described above and further illustrated in the detailed examples following, give compounds of general formula J. Compounds of general formula J can be converted into compounds of general formula (I) by condensation of the enaminoketone with a suitable reagent such as a bis nucleophile such as formamidine or guanidine hydrochloride. For example, reaction of J with a bis nucleophile in the presence of a strong base in an appropriate solvent at temperatures between 30 and 250° C. will give compounds of formula (I). In particular, reaction of J with an guanidine hydrochloride in the presence of sodium ethoxide in refluxing ethanol will give compounds of formula (I).

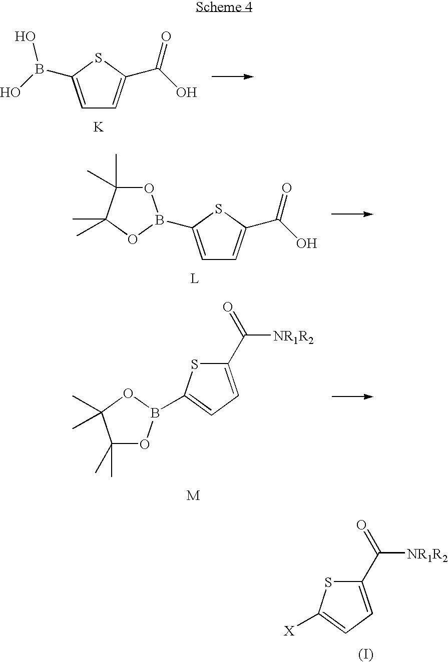

### Scheme 4

Scheme 4 illustrates the use of a thiophene boronate ester in a Suzuki coupling reaction to give compounds of formula (I). Compound L can be prepared from Compound K, 2-carboxy-5-thiopheneboronic acid, by reaction with pinacol in a mixture of tetrahydrofuran:toluene (1:1). Compounds of formula M can be prepared from compounds of formula L by the previously described amide coupling procedure (see Scheme 2 above). Compounds of formula (I) can be prepared from Compounds of formula M using the previously described Suzuki reaction procedure (see Scheme 2 above).

In some instances, the chemistry depicted in schemes 1 and 2 describe the synthesis of compounds of general formula (I) which make use of boronic acids or boronate esters. Many boronic acids and boronate esters are commercially available. When not commercially available, boronic acids and boronate esters may be synthesized by standard methods, including those depicted in scheme 5 (see below). Aryl or heteroaryl boronate esters may be synthesized by reaction of an aryl or heteroaryl halide with bis(pinacolato)diboron and an appropriate palladium catalyst in an appropriate solvent with appropriate additives. For example, reaction of an aryl halide and bis(pinacolato)diboron with PdCl₂(dppf)₂, and potassium acetate, in DMF as solvent at 80° C. for 90 minutes can give boronate esters of general formula P. Aryl or heteroaryl boronic acids may be synthesized by treating an appropriate aryl halide or heteroaryl halide with a strong base such as n-BuLi or t-BuLi in a solvent such as THF or dioxane, followed by reaction of the intermediate organometallic species with a reagent to introduce the boron. For example, reaction of an aryl halide in THF at −70° C. with n-butyl lithium, followed by addition of tri-isopropylborate gives, after standard work up, aryl boronic acids of general formula R.

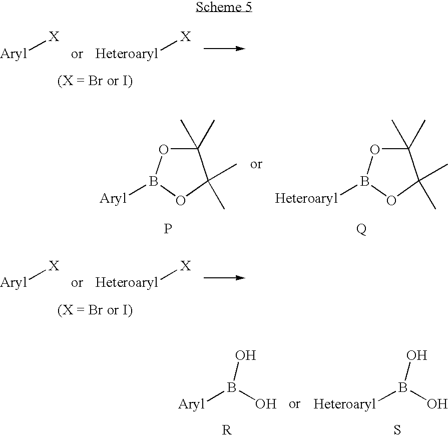

### Scheme 5

Intermediates used in schemes 1, 2, 3 and 4 can be obtained from commercial sources or synthesized by one skilled in the art. Some of the intermediates may be synthesized, for example, by the synthetic sequences outlined in scheme 5 and further detailed in the experimental sections following.

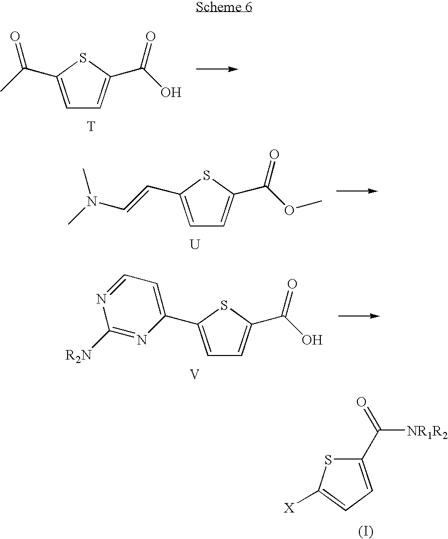

### Scheme 6

Scheme 6 illustrates the use of an alternative procedure to give compounds of formula (I). Compound U can be prepared from Compound T, by heating the acid in DMF-DMA to afford the ester enamine U. Compounds of formula V can be prepared from compounds of formula U by heating the compounds with an appropriately substituted guanidine with a suitable base and solvent such as 2-methoxyethanol. Compounds of formula (I) can be prepared from Compounds of formula V using standard peptide coupling conditions such as EDCI and HOBt in DMF or another appropriate solvent.

As mentioned above, the compounds of the present invention are inhibitors of ROCK activity which are useful in the treatment of disorders associated with inappropriate ROCK activity. Thus, in a further aspect of the present invention, there is provided a compound of formula (I), or a salt, solvate, or a physiologically functional derivative thereof for use in therapy.

In a further aspect of the present invention, there is provided a method of treating a disorder in a mammal, said disorder being mediated by inappropriate ROCK-1 activity, comprising: administering to said mammal a therapeutically effective amount of a compound of formula (I) or a salt, solvate or a physiologically functional derivative thereof.

In a further aspect of the present invention, there is provided the use of a compound of formula (I), or a salt, solvate, or a physiologically functional derivative thereof in the preparation of a medicament for use in the treatment of a disorder mediated by inappropriate ROCK-1 activity.

The term “disorder mediated by inappropriate ROCK-1 activity” includes cardiovascular diseases (such as hypertension, chronic and congestive heart failure, cardiac hypertrophy, restenosis, chronic renal failure and atherosclerosis); asthma, male erectile dysfunctions, female sexual dysfunction and over-active bladder syndrome; neuroinflammatory diseases (such as stroke, multiple sclerosis, Alzheimer's disease, Parkinson's disease, amyotrophic lateral sclerosis and inflammatory pain); other inflammatory diseases (such as rheumatoid arthritis, irritable bowel syndrome and inflammatory bowel disease); spinal cord injury, acute neuronal injury (stroke, traumatic brain injury), Parkinsons disease, Alzheimers disease and other neurodegenerative disorders; cancer and tumor metastasis; viral and bacterial diseases; and diabetes.

While it is possible that, for use in therapy, therapeutically effective amounts of a compound of formula (I), as well as salts, solvates and physiological functional derivatives thereof, may be administered as the raw chemical, it is possible to present the active ingredient as a pharmaceutical composition. Accordingly, the invention further provides a pharmaceutical composition, comprising a therapeutically effective amount of a compound of formula (I), or a physiologically functional derivative thereof and one or more of pharmaceutically acceptable carriers, diluents and excipients. The compounds of the formula (I) and salts, solvates and physiological functional derivatives thereof, are as described above. The carrier(s), diluent(s) or excipient(s) must be acceptable in the sense of being compatible with the other ingredients of the formulation and not deleterious to the recipient thereof. In accordance with another aspect of “the invention there” is also provided a process for the preparation of a pharmaceutical formulation including admixing a compound of the formula (I), or salts, solvates and physiological functional derivatives thereof, with one or more pharmaceutically acceptable carriers, diluents or excipients.

Pharmaceutical formulations may be presented in unit dose forms containing a predetermined amount of active ingredient per unit dose. Such a unit may contain, for example, 0.5 mg to 1 g, preferably 1 mg to 700 mg, more preferably 5 mg to 100 mg of a compound of the formula (I), depending on the condition being treated, the route of administration and the age, weight and condition of the patient, or pharmaceutical formulations may be presented in unit dose forms containing a predetermined amount of active ingredient per unit dose. Preferred unit dosage formulations are those containing a daily dose or sub-dose, as herein above recited, or an appropriate fraction thereof, of an active ingredient. Furthermore, such pharmaceutical formulations may be prepared by any of the methods well known in the pharmacy art.

Pharmaceutical formulations may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such formulations may be prepared by any method known in the art of pharmacy, for example by bringing into association the active ingredient with the carrier(s) or excipient(s).

Pharmaceutical formulations adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

For instance, for oral administration in the form of a tablet or capsule, the active drug component can be combined with an oral, non-toxic pharmaceutically acceptable inert carrier such as ethanol, glycerol, water and the like. Powders are prepared by comminuting the compound to a suitable fine size and mixing with a similarly comminuted pharmaceutical carrier such as an edible carbohydrate, as, for example, starch or mannitol. Flavoring, preservative, dispersing and coloring agent can also be present.

Capsules are made by preparing a powder mixture, as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by wetting with a binder such as syrup, starch paste, acadia mucilage or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of the compound. Syrups can be prepared by dissolving the compound in a suitably flavored aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

Where appropriate, dosage unit formulations for oral administration can be microencapsulated. The formulation can also be prepared to prolong or sustain the release as for example by coating or embedding particulate material in polymers, wax or the like.

The compounds of formula (I), and salts, solvates and physiological functional derivatives thereof, can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The compounds of formula (I) and salts, solvates and physiological functional derivatives thereof may also be delivered by the use of monoclonal antibodies as individual carriers to which the compound molecules are coupled. The compounds may also be coupled with soluble polymers as targetable drug carriers. Such polymers can include polyvinylpyrrolidone, pyran copolymer, polyhydroxypropylmethacrylamide-phenol, polyhydroxyethylaspartamidephenol, or polyethyleneoxidepolylysine substituted with palmitoyl residues. Furthermore, the compounds may be coupled to a class of biodegradable polymers useful in achieving controlled release of a drug, for example, polylactic acid, polepsilon caprolactone, polyhydroxy butyric acid, polyorthoesters, polyacetals, polydihydropyrans, polycyanoacrylates and cross-linked or amphipathic block copolymers of hydrogels.

Pharmaceutical formulations adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6), 318 (1986).

Pharmaceutical formulations adapted for topical administration may be formulated as ointments, creams, suspensions, lotions, powders, solutions, pastes, gels, sprays, aerosols or oils.

For treatments of the eye or other external tissues, for example mouth and skin, the formulations are preferably applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical formulations adapted for topical administrations to the eye include eye drops wherein the active ingredient is dissolved or suspended in a suitable carrier, especially an aqueous solvent.

Pharmaceutical formulations adapted for topical administration in the mouth include lozenges, pastilles and mouth washes.

Pharmaceutical formulations adapted for rectal administration may be presented as suppositories or as enemas.

Pharmaceutical formulations adapted for nasal administration wherein the carrier is a solid include a coarse powder having a particle size for example in the range 20 to 500 microns which is administered in the manner in which snuff is taken, i.e. by rapid inhalation through the nasal passage from a container of the powder held close up to the nose. Suitable formulations wherein the carrier is a liquid, for administration as a nasal spray or as nasal drops, include aqueous or oil solutions of the active ingredient.

Pharmaceutical formulations adapted for administration by inhalation include fine particle dusts or mists, which may be generated by means of various types of metered, dose pressurised aerosols, nebulizers or insufflators.

Pharmaceutical formulations adapted for vaginal administration may be presented as pessaries, tampons, creams, gels, pastes, foams or spray formulations.

Pharmaceutical formulations adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

It should be understood that in addition to the ingredients particularly mentioned above, the formulations may include other agents conventional in the art having regard to the type of formulation in question, for example those suitable for oral administration may include flavouring agents.

A therapeutically effective amount of a compound of the present invention will depend upon a number of factors including, for example, the age and weight of the human or other animal, the precise condition requiring treatment and its severity, the nature of the formulation, and the route of administration, and will ultimately be at the discretion of the attendant physician or veterinarian. However, an effective amount of a compound of formula (I) for the treatment of neoplastic growth, for example colon or breast carcinoma, will generally be in the range of 0.1 to 100 mg/kg body weight of recipient (mammal) per day and more usually in the range of 1 to 10 mg/kg body weight per day. Thus, for a 70 kg adult mammal, the actual amount per day would usually be from 70 to 700 mg and this amount may be given in a single dose per day or more usually in a number (such as two, three, four, five or six) of sub-doses per day such that the total daily dose is the same. An effective amount of a salt or solvate, or physiologically functional derivative thereof, may be determined as a proportion of the effective amount of the compound of formula (I) per se. It is envisaged that similar dosages would be appropriate for treatment of the other conditions referred to above.

### EXAMPLES

As used herein the symbols and conventions used in these processes, schemes and examples are consistent with those used in the contemporary scientific literature, for example, the Journal of the American Chemical Society or the Journal of Biological Chemistry. Standard single-letter or three-letter abbreviations are generally used to designate amino acid residues, which are assumed to be in the L-configuration unless otherwise noted. Unless otherwise noted, all starting materials were obtained from commercial suppliers and used without further purification. Specifically, the following abbreviations may be used in the examples and throughout the specification:

| | |
|---|---|
| g (grams); | mg (milligrams); |
| L (liters); | mL (milliliters); |
| μL (microliters); | psi (pounds per square inch); |
| M (molar); | mM (millimolar); |
| i.v. (intravenous); | Hz (Hertz); |
| MHz (megaHertz); | mol (moles); |
| mmol (millimoles); | rt (room temperature); |
| min (minutes); | h (hours); |
| mp (melting point); | TLC (thin layer chromatography); |
| Tᵣ (retention time); | RP (reverse phase); |
| MeOH (methanol); | i-PrOH (isopropanol); |
| TEA (triethylamine); | TFA (trifluoroacetic acid); |
| TFAA (trifluoroacetic anhydride); | THF (tetrahydrofuran); |
| DMSO (dimethylsulfoxide); | AcOEt (ethyl acetate); |
| DME (1,2-dimethoxyethane); | DCM (dichloromethane); |
| DCE (dichloroethane); | DMF (N,N-dimethylformamide); |
| DMPU (N,N′-dimethylpropyleneurea); | CDI (1,1′-carbonyldiimidazole); |
| IBCF (isobutyl chloroformate); | HOAc (acetic acid); |
| HOSu (N-hydroxysuccinimide); | HOBT (1-hydroxybenzotriazole); |
| mCPBA (meta-chloroperbenzoic acid); | CBZ (benzyloxycarbonyl); |
| EDC (1-[(3-dimethylamino)propyl]-3- | atm (atmosphere); |
| ethylcarbodiimide hydrochloride); | TMS (trimethylsilyl); |
| BOC (tert-butyloxycarbonyl); FMOC | TBS (t-butyldimethylsilyl); |
| (9-fluorenylmethoxycarbonyl); | BSA (bovine serum albumin) |
| DCC (dicyclohexylcarbodiimide); | HRP (horseradish peroxidase); |
| Ac (acetyl); | |
| TMSE (2-(trimethylsilyl)ethyl); | |
| TIPS (triisopropylsilyl); | |
| DMAP (4-dimethylaminopyridine); | |
| ATP (adenosine triphosphate); | |
| DMEM (Dulbecco′s modified Eagle | |
| medium); | |
| HPLC (high pressure liquid | |
| chromatography); | |
| BOP (bis(2-oxo-3-oxazolidinyl)phosphinic | |
| chloride); | |
| TBAF (tetra-n-butylammonium fluoride); | |
| HBTU(O-Benzotriazole-1-yl-N,N,N′,N′- | |
| tetramethyluroniumhexafluoro phosphate). | |
| HEPES (4-(2-hydroxyethyl)-1-piperazine | |
| ethane sulfonic acid); | |
| DPPA (diphenylphosphoryl azide); | |
| fHNO₃ (fuming HNO₃); and | |
| EDTA (ethylenediaminetetraacetic acid). | |

All references to ether are to diethyl ether; brine refers to a saturated aqueous solution of NaCl. Unless otherwise indicated, all temperatures are expressed in ° C. (degrees Centigrade). All reactions are conducted under an inert atmosphere at room temperature unless otherwise noted.

¹H NMR spectra were recorded on a Varian VXR-300, a Varian Unity-300, a Varian Unity-400 instrument, a Brucker AVANCE-400, or a General Electric QE-300. Chemical shifts are expressed in parts per million (ppm, δ units). Coupling constants are in units of Hertz (Hz). Splitting patterns describe apparent multiplicities and are designated as s (singlet), d (doublet), t (triplet), q (quartet), quint (quintet), m (multiplet), br (broad).

HPLC were recorded on a Gilson HPLC or Shimadzu HPLC system by the following conditions. Column: 50×4.6 mm (id) stainless steel packed with 5 μm Phenomenex Luna C-18; Flow rate: 2.0 mL/min; Mobile phase: A phase=50 mM ammonium acetate (pH 7.4), B phase=acetonitrile, 0-0.5 min (A: 100%, B: 0%), 0.5-3.0 min (A: 100-0%, B: 0-100%), 3.0-3.5 min (A: 0%, B: 100%), 3.5-3.7 min (A: 0-100%, B: 100-0%), 3.7-4.5 min LA: 100%, B: 0%); Detection: UV 254 nm; Injection volume: 3 μL.

Low-resolution mass spectra (MS) were recorded on a JOEL JMS-AX505HA, JOEL SX-102, or a SCIEX-APIiii spectrometer; LC-MS were recorded on a micromass 2MD and Waters 2690; high resolution MS were obtained using a JOEL SX-102A spectrometer. All mass spectra were taken under electrospray ionization (ESI), chemical ionization (CI), electron impact (EI) or by fast atom bombardment (FAB) methods. Infrared (IR) spectra were obtained on a Nicolet 510 FT-IR spectrometer using a 1-mm NaCl cell. Most of the reactions were monitored by thin-layer chromatography on 0.25 mm E. Merck silica gel plates (60F-254), visualized with UV light, 5% ethanolic phosphomolybdic acid or p-anisaldehyde solution. Flash column chromatography was performed on silica gel (230-400 mesh, Merck).

### Example 1

### Method A (see Scheme 1)

### N-(2-phenylethyl)-5-(4-pyridinyl)-2-thiophenecarboxamide

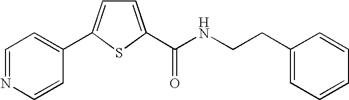

### (a) Preparation of 5-bromo-2-thiophenecarbonyl chloride

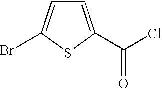

Thionyl chloride (5.01 mL, 68.7 mmol) was added to a mixture of 5-bromo-2-thiophenecarboxylic acid (4.00 g, 22.9 mmol) in toluene (100 mL). The mixture was refluxed for 4 h, filtered hot through a plug of glass wool, and the filtrate was concentrated to dryness by rotary evaporation. A light amber oil crystallized upon cooling to give 5-bromo-2-thiophenecarbonyl chloride (4.02 g) as a light tan solid.

1H NMR (400 MHz, CDCl₃) δ ppm 7.16 (d, J=4.2 Hz, 1H), 7.72 (d, J=4.2 Hz, 1H).

### (b) Preparation of 5-bromo-N-(2-phenylethyl)-2-thiophenecarboxamide

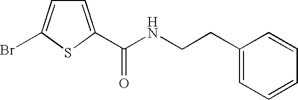

A solution of 2-phenethylamine (55.6 μL, 0.443 mmol), TEA (61.7 μL, 0.443 mmol) and THF (1.0 mL) was added to a solution of 5-bromo-2-thiophenecarbonyl chloride (100 mg, 0.443 mmol) in THF (1.0 mL). The reaction was placed on a shaker table for 18 h and partitioned between AcOEt (10 mL) and water (1.0 mL). The phases were separated and the aqueous phase extracted with AcOEt (2×10 mL). Saturated brine (1.0 mL) was used to break up an emulsion. The combined organic phase was dried (MgSO₄) and concentrated to dryness to give 5-bromo-N-(2-phenylethyl)-2-thiophenecarboxamide (107 mg) as a white solid and used without purification in the next step.

1H NMR (400 MHz, DMSO-d6) δ ppm 2.82 (pseudo t, J=7.4 Hz, 2H), 3.43 (dd, J=6.5 Hz, 2H), 7.18-7.31 (m, 6H), 7.54 (d, J=4.0 Hz, 1H) 8.67 (t, J=5.5 Hz, 1H); MS m/z 310/312 (M+1)⁺.

### (c) Preparation of N-(2-phenylethyl)-5-(4-pyridinyl)-2-thiophenecarboxamide

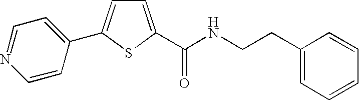

A mixture of 5-bromo-N-(2-phenylethyl)-2-thiophenecarboxamide (114 mg, 0.367 mmol) and DME (2.0 mL) was added to a mixture of 4-pyridylboronic acid (88 mg, 0.716 mmol), 2M aq. sodium carbonate (0.358 mL, 0.716 mmol), dichlorobis(triphenylphosphine)palladium(II) (17 mg, 0.024 mmol) and DME (1.0 mL) in a 2-5 mL Emrys™ Process Vial from Personal Chemistry. The vial was capped and heated in a Personal Chemistry Creator™ microwave instrument at 175° C. for 10 min. The reaction was diluted with water (1.0 mL) and extracted with AcOEt (2×10 mL). The combined organic phase was dried over MgSO₄ and the volatiles removed in the presence of 0.5-1.0 g of silica gel 60 (40-63μ). The pre-adsorbed material was chromatographed using a pre-packed ISCO silica gel cartridge (4 gram) and eluted with a hexane: AcOEt linear solvent gradient (0% to 100% AcO Et) to give N-(2-phenylethyl)-5-(4-pyridinyl)-2-thiophenecarboxamide (37 mg) as an off-white solid. 1H NMR (300 MHz, DMSO-d6) δ ppm 2.88 (pseudo t, J=7.4 Hz, 2H), 3.50 (dd, J=6-4 Hz, 2H), 7.22-7.45 (m, 5H), 7.72/8.64 (AB q, 4H), 7.80 (d, J=3.9 Hz, 1H) 7.84 (d, J=4.1 Hz, 1H), 8.77 (t, J=5.6 Hz, 1H); MS m/z 309 (M+1)⁺.

### Method B (see Scheme 2)

### N-(2-phenylethyl)-5-(4-pyridinyl)-2-thiophenecarboxamide

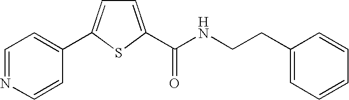

### (a) Preparation of methyl 5-bromo-2-thiophenecarboxylate

A mixture of 5-bromo-2-thiophenecarboxylic acid (5.00 g, 28.6 mmol), cesium carbonate (14.0 g, 42.9 mmol) and acetonitrile (100 mL) was stirred for several minutes before adding methyl iodide (8.90 mL, 143 mmol). The reaction was refluxed for 2 h and then stirred at rt for 64 h. The reaction mixture was filtered and the filtrate was reduced to dryness to give methyl 5-bromo-2-thiophenecarboxylate (4.61 g) as a white solid.

1H NMR (400 MHz, DMSO-d6) δ ppm 3.82 (s, 3H), 7.38 (d, J=4.0 Hz, 1H), 7.64 (d, J=4.1 Hz, 1H).

### (b) Preparation of 5-(4-pyridinyl)-2-thiophenecarboxylic acid

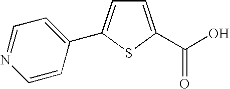

A mixture of methyl 5-bromo-2-thiophenecarboxylate (4.61 g, 20.9 mmol), 4-pyridylboronic acid (3.86 g, 31.4 mmol), 2M aq. sodium carbonate (15.7 mL, 31.4 mmol), dichlorobis(triphenylphosphine)palladium(II) (0.730 g, 1.04 mmol) and DME (100 mL) was refluxed for 3 days. The cooled reaction mixture was treated with 0.5M aq. NaOH (100 mL) and diluted with MeOH (200 mL). Decanted liquid portion trough a pad of Celite, then treated remaining solids with 0.5M aq. NaOH (100 mL) using heat and sonication to complete the dissolution. Filtered remaining mixture through Celite and the combined filtrate was concentrated to remove the excess MeOH. The aqueous mixture was acidified to pH 4 with conc. HCl. The solids were collected by suction filtration and dried to a cake. The cake was triturated, then sonicated in a mixture of ether (150 mL) and MeOH (15 mL). The solids were collected by suction filtration and dried to give 5-(4-pyridinyl)-2-thiophenecarboxylic acid (4.31 g) as an off-white powder.

1H NMR (400 MHz, DMSO-d6) δ ppm 7.75/8.64 (AB q, J=6.1 Hz, 4H), 7.79 (d, J=3.8 Hz, 1H), 7.86 (d, J=4.0 Hz, 1H), 13.39 (br s, 1H).

### (c) Preparation of N-(2-phenylethyl)-5-(4-pyridinyl)-2-thiophenecarboxamide

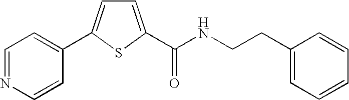

5-(4-Pyridinyl)-2-thiophenecarboxylic acid (20.5 mg, 0.10 mmol) was added to a Bohdan filter cartridge containing Argonaut polystyrene-supported carbodiimide resin (208 mg, 0.20 mmol; 0.96 mmoles/g loading), HOBt (20 mg, 0.15 mmol), and DMF (3.0 mL) and the mixture was shaken for 215 min. The 2-phenethylamine (10.7 μL, 0.085 mmol) was added to the reaction and shaking continued for 18 h at rt. Next, Argonaut MP-carbonate resin (208 mg, 0.50 mmol) was added to scavenge the excess carboxylic acid and HOBt and shaking continued for 4 h. The filtrate was drained from the cartridge and DMF (2.0 mL) was added to the cartridge containing the resins. After shaking for 30 min. the filtrate was again drained and the combined filtrates were concentrated to dryness under high vacuum with heating. The residue was purified by using a pre-packed ISCO silica gel cartridge (4 gram) and eluted with a hexane: AcOEt linear solvent gradient (0% to 100% AcOEt) to give N-(2-phenylethyl)-5-(4-pyridinyl)-2-thiophenecarboxamide (13 mg) as an white solid.

1H NMR (300 MHz, DMSO-d6) δ ppm 2.88 (pseudo t, J=7.4 Hz, 2H), 3.50 (dd, J=6.3 Hz, 2H), 7.22-7.36 (m, 5H), 7.72/8.64 (AB q, J=6.6 Hz, 4H), 7.81 (d, J=3.9 Hz, 1H), 7.84 (d, J=4.1 Hz, 1H), 8.77 (t, J=5.6 Hz, 1H); MS m/z 309 (M+1)⁺.

### Example 2

### N-(3-methoxybenzyl)-5-(4-pyridinyl)-2-thiophenecarboxamide

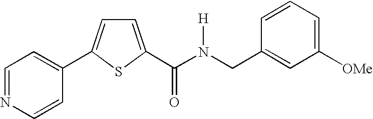

### (a) Preparation of 5-bromo-N-(3-methoxybenzyl)-2-thiophenecarboxamide

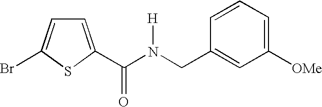

A mixture of 5-bromo-2-thiophenecarboxylic acid (0.436 g, 2.49 mmol), HOBt (0.404 g, 2.99 mmol), EDC (0.573 g, 2.99 mmol) and DMF (10 mL) was stirred for 1 h at rt. Next, 3-methoxybenzylamine (0.351 mL, 2.74 mmol) was added and the reaction was stirred for 20 h at rt. The DMF was removed by rotary evaporation under reduced pressure and the oil was partitioned between AcOEt: water (50 mL: 10 mL). The phases were separated and the aqueous phase was extracted with AcOEt (25 mL). The combined organic layer was washed with 1N aq. sodium hydroxide (3×10 mL), water (2×10 mL), and then dried (MgSO₄) for 20 hours. The volatiles were removed to give 5-bromo-N-(3-methoxybenzyl)-2-thiophenecarbox-amide (0.584 g) as a yellow oil.

1H NMR (400 MHz, DMSO-d6) δ ppm 3.71 (s, 3H), 4.38 (d, J=5.8 Hz, 2H), 6.79-6.86 (m, 3H), 7.23 (t, J=8.0 Hz, 1H), 7.28 (d, J=4.0 Hz, 1H), 7.62 (d, J=4.0 Hz, 1H), 9.09 (t, J=5.9 Hz, 1H); MS m/z 326/328 (M+1)⁺.

### (b) Preparation of N-(3-methoxybenzyl)-5-(4-pyridinyl)-2-thiophenecarboxamide

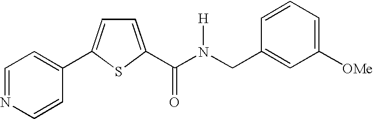

A mixture of 5-bromo-N-(3-methoxybenzyl)-2-thiophenecarboxamide (150 mg, 0.460 mmol), 4-pyridylboronic acid (85 mg, 0.690 mmol), 2M aq. sodium carbonate (0.345 mL, 0.690 mmol), dichlorobis(triphenylphosphine)palladium(II) (16 mg, 0.023 mmol), DME (2.0 mL) and EtOH (1.0 mL) was placed into a 2-5 mL Emrys™ Process Vial from Personal Chemistry. The vial was capped and heated in a Personal Chemistry Creator™ microwave Instrument at 175° C. for 10 min. The reaction mixture was diluted with water (5.0 mL) and extracted with AcOEt (25 mL). The phases were separated and the organic layer was washed with water (5.0 mL). The organic layer was dried over MgSO₄ and the volatiles removed in the presence of 1.0 g of silica gel 60 (40-63μ). The pre-adsorbed material was chromatographed using a pre-packed ISCO silica gel cartridge (4 gram) and eluted with a hexane: AcOEt linear solvent gradient (0% to 100% AcOEt) to give N-(3-methoxybenzyl)-5-(4-pyridinyl)-2-thiophenecarboxamide (68 mg) as a yellow-white solid.

1H NMR (300 MHz, DMSO-d6) δ ppm 3.77 (s, 3H), 4.48 (d, J=5.9 Hz, 2H), 6.85-6.94 (m, 3H), 7.29 (t, J=8.0 Hz, 1H), 7.73/8.65 (AB q, J=6.0 Hz, 4H), 7.86 (d, J=4.0 Hz, 1H), 7.90 (d, J=3.9 Hz, 1H), 9.20 (t, J=5.8 Hz, 1H); MS m/z 325 (M+1)⁺.

### Example 3

### 5-(4-pyridinyl)-N-(2-pyridinylmethyl)-2-thiophenecarboxamide

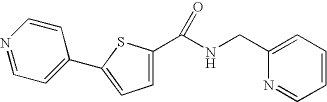

Prepared In a similar manner as described for Example 1b Method B from 5-(4-pyridinyl)-2-thiophenecarboxylic acid and 2-pyridinylmethanamine. The reaction mixture was purified by reversed-phase silica gel chromatography using a LC-MS mass-triggered fraction collection procedure to give 5-(4-pyridinyl)-N-(2-pyridinyl-methyl)-2-thiophenecarboxamide (9.7 mg) as a solid.

MS m/z 296 (M+1)⁺.

### Example 4

### N-(1-naphthylmethyl)-5-(4-pyridinyl)-2-thiophenecarboxamide

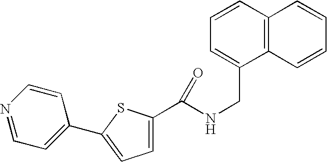

Prepared in a similar manner as described for Example 3 from 5-(4-pyridinyl)-2-thiophenecarboxylic acid and 1-naphthylmethanamine to give N-(1-naphthyl-methyl)-5-(4-pyridinyl)-2-thiophenecarboxamide (10.4 mg) as a solid.

MS m/z 345 (M+1)⁺.

### Example 5

### N-(2-ethoxybenzyl)-5-(4-pyridinyl)-2-thiophenecarboxamide

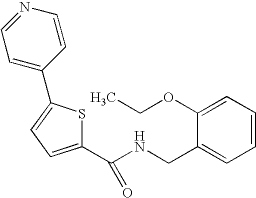

Prepared in a similar manner as described for Example 3 from 5-(4-pyridinyl)-2-thiophenecarboxylic acid and (2-ethoxyphenyl)methanamine to give N-(2-ethoxy-benzyl)-5-(4-pyridinyl)-2-thiophenecarboxamide (12 mg) as a solid.

MS m/z 339 (M+1)⁺.

### Example 6

### N-(2-bromobenzyl)-5-(4-pyridinyl)-2-thiophenecarboxamide

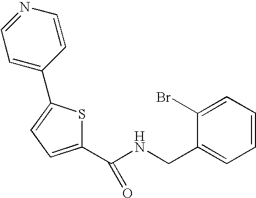

Prepared in a similar manner as described for Example 3 from 5-(4-pyridinyl)-2-thiophenecarboxylic acid and (2-bromophenyl)methanamine to give N-(2-bromo-benzyl)-5-(4-pyridinyl)-2-thiophenecarboxamide (13 mg) as a solid.

MS m/z 373/375 (M+1)⁺.

### Example 7

### N-(2-fluorobenzyl)-5-(4-pyridinyl)-2-thiophenecarboxamide

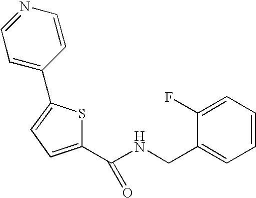

Prepared in a similar manner as described for Example 3 from 5-(4-pyridinyl)-2-thiophenecarboxylic acid and (2-fluorophenyl)methanamine to give N-(2-fluoro-benzyl)-5-(4-pyridinyl)-2-thiophenecarboxamide (11 mg) as a solid.

MS m/z 313 (M+1)⁺.

### Example 8

### N-(2-chlorobenzyl)-5-(4-pyridinyl)-2-thiophenecarboxamide

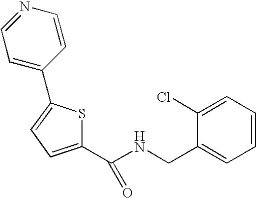

Prepared in a similar manner as described for Example 3 from 5-(4-pyridinyl)-2-thiophenecarboxylic acid and (2-chlorophenyl)methanamine to give N-(2-chloro-benzyl)-5-(4-pyridinyl)-2-thiophenecarboxamide (11 mg) as a solid.

MS m/z 329 (M+1)⁺.

### Example 9

### N-(2-methylbenzyl)-5-(4-pyridinyl)-2-thiophenecarboxamide

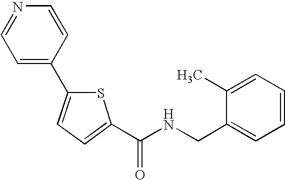

Prepared in a similar manner as described for Example 3 from 5-(4-pyridinyl)-2-thiophenecarboxylic acid and (2-methylphenyl)methanamine to give N-(2-methyl-benzyl)-5-(4-pyridinyl)-2-thiophenecarboxamide (10 mg) as a solid.

MS m/z 309 (M+1)⁺.

### Example 10

### N-(2-trifluoromethylbenzyl)-5-(4-pyridinyl)-2-thiophenecarboxamide

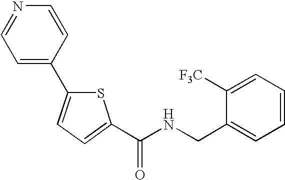

Prepared in a similar manner as described for Example 3 from 5-(4-pyridinyl)-2-thiophenecarboxylic acid and (2-trifluoromethylphenyl)methanamine to give N-(2-trifluoromethylbenzyl)-5-(4-pyridinyl)-2-thiophenecarboxamide (19 mg) as a solid.

MS m/z 363 (M+1)⁺.

### Example 11

### N-(2-trifluoromethoxybenzyl)-5-(4-pyridinyl)-2-thiophenecarboxamide

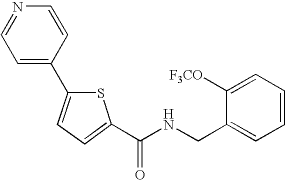

Prepared in a similar manner as described for Example 3 from 5-(4-pyridinyl)-2-thiophenecarboxylic acid and (2-trifluoromethoxyphenyl)methanamine to give N-(2-trifluoromethoxybenzyl)-5-(4-pyridinyl)-2-thiophenecarboxamide (13 mg) as a solid.

MS m/z 379 (M+1)⁺.

### Example 12

### N-(3-trifluoromethylbenzyl)-5-(4-pyridinyl)-2-thiophenecarboxamide

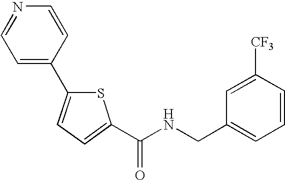

Prepared in a similar manner as described for Example 3 from 5-(4-pyridinyl)-2-thiophenecarboxylic acid and (3-trifluoromethylphenyl)methanamine to give N-(3-trifluoromethylbenzyl)-5-(4-pyridinyl)-2-thiophenecarboxamide (12 mg) as a solid.

MS m/z 363 (M+1)⁺.

### Example 13

### N-(3-fluorobenzyl)-5-(4-pyridinyl)-2-thiophenecarboxamide

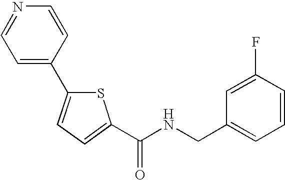

Prepared in a similar manner as described for Example 3 from 5-(4-pyridinyl)-2-thiophenecarboxylic acid and (3-fluorophenyl)methanamine to give N-(3-fluorobenzyl)-5-(4-pyridinyl)-2-thiophenecarboxamide (12 mg) as a solid.

MS m/z 313 (M+1)⁺.

### Example 14

### N-(3-chlorobenzyl)-5-(4-pyridinyl)-2-thiophenecarboxamide

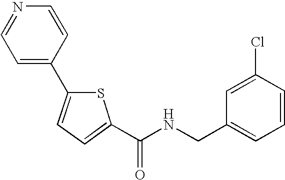

Prepared in a similar manner as described for Example 3 from 5-(4-pyridinyl)-2-thiophenecarboxylic acid and (3-chlorophenyl)methanamine to give N-(3-chlorobenzyl)-5-(4-pyridinyl)-2-thiophenecarboxamide (13 mg) as a solid.

MS m/z 329 (M+1)⁺.

### Example 15

### N-(3-bromobenzyl)-5-(4-pyridinyl)-2-thiophenecarboxamide

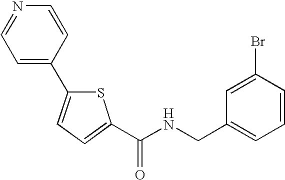

Prepared in a similar manner as described for Example 3 from 5-(4-pyridinyl)-2-thiophenecarboxylic acid and (3-bromophenyl)methanamine to give N-(3-bromobenzyl)-5-(4-pyridinyl)-2-thiophenecarboxamide (13 mg) as a solid.

MS m/z 373/375 (M+1)⁺.

### Example 16

### N-(3-iodobenzyl)-5-(4-pyridinyl)-2-thiophenecarboxamide

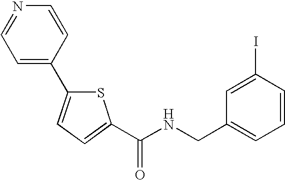

Prepared in a similar manner as described for Example 3 from 5-(4-pyridinyl)-2-thiophenecarboxylic acid and (3-iodophenyl)methanamine to give N-(3-iodobenzyl)-5-(4-pyridinyl)-2-thiophenecarboxamide (13 mg) as a solid.

MS m/z 421 (M+1)⁺.

### Example 17

### N-(3-methylbenzyl)-5-(4-pyridinyl)-2-thiophenecarboxamide

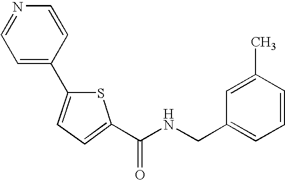

Prepared in a similar manner as described for Example 3 from 5-(4-pyridinyl)-2-thiophenecarboxylic acid and (3-methylphenyl)methanamine to give N-(3-methylbenzyl)-5-(4-pyridinyl)-2-thiophenecarboxamide (9.2 mg) as a solid.

MS m/z 309 (M+1)⁺.

### Example 18

### N-(3-methoxybenzyl)-5-(4-pyridinyl)-2-thiophenecarboxamide

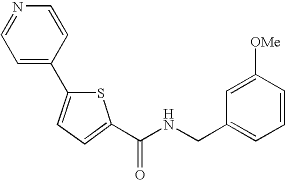

Prepared in a similar manner as described for Example 3 from 5-(4-pyridinyl)-2-thiophenecarboxylic acid and (3-methoxyphenyl)methanamine to give N-(3-methoxybenzyl)-5-(4-pyridinyl)-2-thiophenecarboxamide (8.8 mg) as a solid.

MS m/z 325 (M+1)⁺.

### Example 19

### N-(3-trifluoromethoxybenzyl)-5-(4-pyridinyl)-2-thiophenecarboxamide

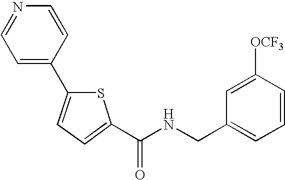

Prepared in a similar manner as described for Example 3 from 5-(4-pyridinyl)-2-thiophenecarboxylic acid and (3-trifluoromethoxyphenyl)methanamine to give N-(3-trifluoromethoxybenzyl)-5-(4-pyridinyl)-2-thiophenecarboxamide (15 mg) as a solid.

MS m/z 379 (M+1)⁺.

### Example 20

### N-(3-phenoxybenzyl)-5-(4-pyridinyl)-2-thiophenecarboxamide

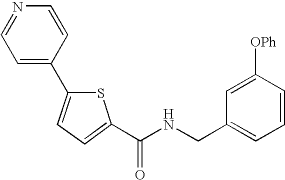

Prepared in a similar manner as described for Example 3 from 5-(4-pyridinyl)-2-thiophenecarboxylic acid and (3-phenoxyphenyl)methanamine to give N-(3-phenoxybenzyl)-5-(4-pyridinyl)-2-thiophenecarboxamide (12 mg) as a solid.

MS m/z 387 (M+1)⁺.

### Example 21

### N-(4-fluorobenzyl)-5-(4-pyridinyl)-2-thiophenecarboxamide

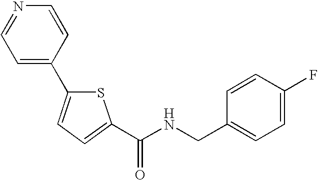

Prepared in a similar manner as described for Example 3 from 5-(4-pyridinyl)-2-thiophenecarboxylic acid and (4-fluorophenyl)methanamine to give N-(4-fluorobenzyl)-5-(4-pyridinyl)-2-thiophenecarboxamide (11 mg) as a solid.

MS m/z 313 (M+1)⁺.

### Example 22

### N-(4-bromobenzyl)-5-(4-pyridinyl)-2-thiophenecarboxamide

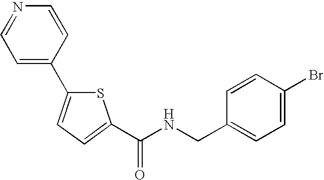

Prepared in a similar manner as described for Example 3 from 5-(4-pyridinyl)-2-thiophenecarboxylic acid and (4-bromophenyl)methanamine to give N-(4-bromobenzyl)-5-(4-pyridinyl)-2-thiophenecarboxamide (11 mg) as a solid.

MS m/z 373/375 (M+1)⁺.

### Example 23

### N-(4-iodobenzyl)-5-(4-pyridinyl)-2-thiophenecarboxamide

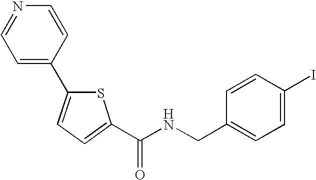

Prepared in a similar manner as described for Example 3 from 5-(4-pyridinyl)-2-O thiophenecarboxylic acid and (4-iodophenyl)methanamine to give N-(4-iodobenzyl)-5-(4-pyridinyl)-2-thiophenecarboxamide (4.2 mg) as a solid.

MS m/z 421 (M+1)⁺.

### Example 24

### N-(4-trifluoromethylbenzyl)-5-(4-pyridinyl)-2-thiophenecarboxamide

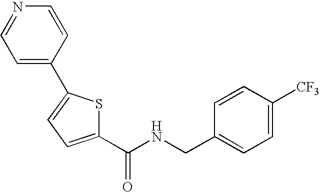

Prepared in a similar manner as described for Example 3 from 5-(4-pyridinyl)-2-thiophenecarboxylic acid and (4-trifluoromethylphenyl)methanamine to give N-(4-trifluoromethylbenzyl)-5-(4-pyridinyl)-2-thiophenecarboxamide (15 mg) as a solid.

MS m/z 363 (M+1)⁺.

### Example 25

### N-(4-methylbenzyl)-5-(4-pyridinyl)-2-thiophenecarboxamide

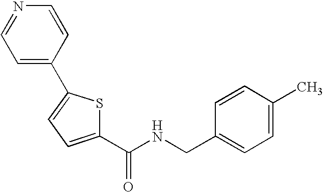

Prepared in a similar manner as described for Example 3 from 5-(4-pyridinyl)-2-thiophenecarboxylic acid and (4-methylphenyl)methanamine to give N-(4-methylbenzyl)-5-(4-pyridinyl)-2-thiophenecarboxamide (3.9 mg) as a solid.

MS m/z 309 (M+1)⁺.

### Example 26

### N-(4-trifluoromethoxybenzyl)-5-(4-pyridinyl)-2-thiophenecarboxamide

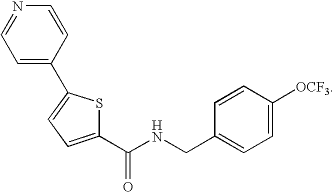

Prepared in a similar manner as described for Example 3 from 5-(4-pyridinyl)-2-thiophenecarboxylic acid and (4-trifluoromethoxyphenyl)methanamine to give N-(4-trifluoromethoxybenzyl)-5-(4-pyridinyl)-2-thiophenecarboxamide (14 mg) as a solid.

MS m/z 379 (M+1)⁺.

### Example 27

### N-[4-(aminosulfonyl)benzyl-5-(4-pyridinyl]-2-thiophenecarboxamide

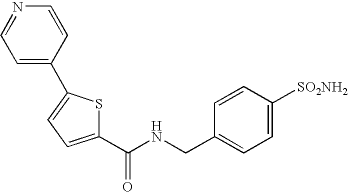

Prepared in a similar manner as described for Example 3 from 5-(4-pyridinyl)-2-thiophenecarboxylic acid and 4-(aminomethyl)benzenesulfonamide to give N-[4-(aminosulfonyl)benzyl]-5-(4-pyridinyl)-2-thiophenecarboxamide (8.0 mg) as a solid.

MS m/z 374 (M+1)⁺.

### Example 28

### N-[4-(methylsulfonyl)benzyl]-5-(4-pyridinyl)-2-thiophenecarboxamide

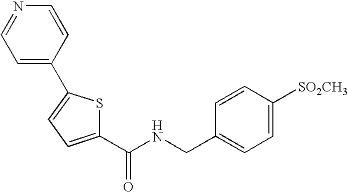

Prepared in a similar manner as described for Example 3 from 5-(4-pyridinyl)-2-thiophenecarboxylic acid and [4-(methylsulfonyl)phenyl]methanamine to give N-[4-(methylsulfonyl)benzyl]-5-(4-pyridinyl)-2-thiophenecarboxamide (13 mg) as a solid.

MS m/z 373 (M+1)⁺.

### Example 29

### 5-(4-pyridinyl)-N-(4-pyridinylmethyl)-2-thiophenecarboxamide

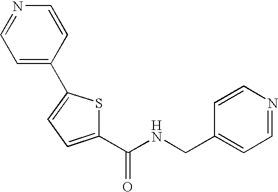

Prepared in a similar manner as described for Example 3 from 5-(4-pyridinyl)-2-thiophenecarboxylic acid and 4-pyridinylmethanamine to give 5-(4-pyridinyl)-N-(4-pyridinylmethyl)-2-thiophenecarboxamide (14 mg) as a solid.

MS m/z 296 (M+1)⁺.

### Example 30

### N-benzyl-N-methyl-5-(4-pyridinyl)-2-thiophenecarboxamide

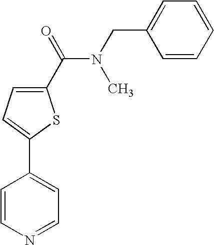

Prepared in a similar manner as described for Example 2a from 5-(4-pyridinyl)-2-thiophenecarboxylic acid and N-methyl(phenyl)methanamine to give N-benzyl-N-methyl-5-(4-pyridinyl)-2-thiophenecarboxamide (23 mg) as a solid.

MS m/z 309 (M+1)⁺.

### Example 31

### 5-(4-pyridinyl)-N-[4-(1,2,3-thiadiazol-4-yl)benzyl]-2-thiophenecarboxamide

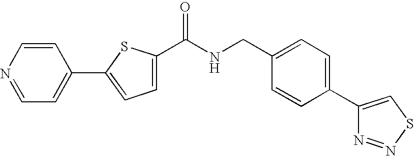

Prepared in a similar manner as described for Example 3 from 5-(4-pyridinyl)-2-thiophenecarboxylic acid and 4-pyridinylmethanamine to give 5-(4-pyridinyl)-N-[4-(1,2,3-thiadiazol-4-yl)benzyl]-2-thiophenecarboxamide (7.3 mg) as a solid.

MS m/z 381 (M+1)⁺.

### Example 32

### 5-(4-pyridinyl)-N-(3-pyridinylmethyl)-2-thiophenecarboxamide

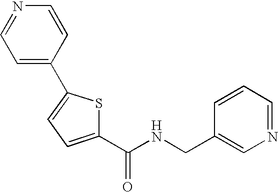

Prepared in a similar manner as described for Example 3 from 5-(4-pyridinyl)-2-thiophenecarboxylic acid and 3-pyridinylmethanamine to give 5-(4-pyridinyl)-N-(3-pyridinylmethyl)-2-thiophenecarboxamide (6.7 mg) as a solid.

MS m/z 296 (M+1)⁺.

### Example 33

### N-[2-(2-methylphenyl)ethyl]-5-(4-pyridinyl)-2-thiophenecarboxamide

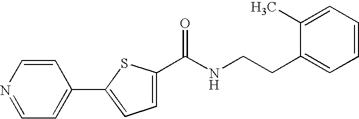

Prepared in a similar manner as described for Example 3 from 5-(4-pyridinyl)-2-thiophenecarboxylic acid and 2-(2-methylphenyl)ethanamine to give N-[2-(2-methylphenyl)ethyl]-5-(4-pyridinyl)-2-thiophenecarboxamide (9.9 mg) as a solid.

MS m/z 323 (M+1)⁺.

### Example 34

### N-[2-(3-methylphenyl)ethyl]-5-(4-pyridinyl)-2-thiophenecarboxamide

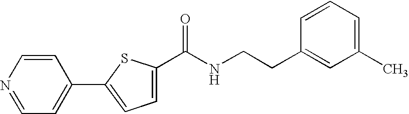

Prepared in a similar manner as described for Example 3 from 5-(4-pyridinyl)-2-thiophenecarboxylic acid and 2-(3-methylphenyl)ethanamine to give N-[2-(3-methylphenyl)ethyl]-5-(4-pyridinyl)-2-thiophenecarboxamide (11 mg) as a solid.

MS m/z 323 (M+1)⁺.

### Example 35

### N-[2-(4-methylphenyl)ethyl]-5-(4-pyridinyl)-2-thiophenecarboxamide

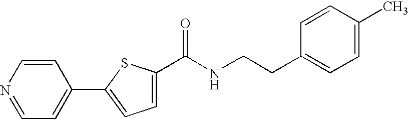

Prepared in a similar manner as described for Example 3 from 5-(4-pyridinyl)-2-thiophenecarboxylic acid and 2-(4-methylphenyl)ethanamine to give N-[2-(4-methylphenyl)ethyl]-5-(4-pyridinyl)-2-thiophenecarboxamide (5.5 mg) as a solid.

MS m/z 323 (M+1)⁺.

### Example 36

### N-[2-(2-fluorophenyl)ethyl]-5-(4-pyridinyl)-2-thiophenecarboxamide

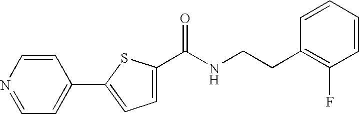

Prepared in a similar manner as described for Example 3 from 5-(4-pyridinyl)-2-thiophenecarboxylic acid and 2-(2-fluorophenyl)ethanamine to give N-[2-(2-fluorophenyl)ethyl]-5-(4-pyridinyl)-2-thiophenecarboxamide (11 mg) as a solid.

MS m/z 327 (M+1)⁺.

### Example 37

### N-[2-(3-fluorophenyl)ethyl]-5-(4-pyridinyl)-2-thiophenecarboxamide

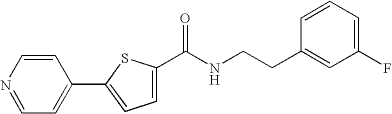

Prepared in a similar manner as described for Example 3 from 5-(4-pyridinyl)-2-thiophenecarboxylic acid and 2-(3-fluorophenyl)ethanamine to give N-[2-(3-fluorophenyl)ethyl]-5-(4-pyridinyl)-2-thiophenecarboxamide (13 mg) as a solid.

MS m/z 327 (M+1)⁺.

### Example 38

### N-[2-(4-fluorophenyl)ethyl]-5-(4-pyridinyl)-2-thiophenecarboxamide

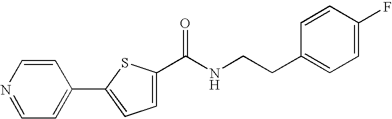

Prepared in a similar manner as described for Example 3 from 5-(4-pyridinyl)-2-thiophenecarboxylic acid and 2-(4-fluorophenyl)ethanamine to give N-[2-(4-fluorophenyl)ethyl]-5-(4-pyridinyl)-2-thiophenecarboxamide (12 mg) as a solid.

MS m/z 327 (M+1)⁺.

### Example 39

### N-[2-(2-methoxyphenyl)ethyl]-5-(4-pyridinyl)-2-thiophenecarboxamide

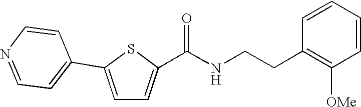

Prepared in a similar manner as described for Example 3 from 5-(4-pyridinyl)-2-thiophenecarboxylic acid and 2-(2-methoxyphenyl)ethanamine to give N-[2-(2-methoxyphenyl)ethyl]-5-(4-pyridinyl)-2-thiophenecarboxamide (7.7 mg) as a solid.

MS m/z 339 (M+1)⁺.

### Example 40

### N-[2-(3-methoxyphenyl)ethyl]-5-(4-pyridinyl)-2-thiophenecarboxamide

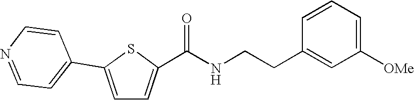

Prepared in a similar manner as described for Example 3 from 5-(4-pyridinyl)-2-thiophenecarboxylic acid and 2-(3-methoxyphenyl)ethanamine to give N-[2-(3-methoxyphenyl)ethyl]-5-(4-pyridinyl)-2-thiophenecarboxamide (7.1 mg) as a solid.

MS m/z 339 (M+1)⁺.

### Example 41

### N-[2-(4-methoxyphenyl)ethyl]-5-(4-pyridinyl)-2-thiophenecarboxamide

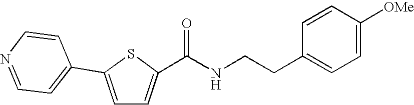

Prepared in a similar manner as described for Example 3 from 5-(4-pyridinyl)-2-thiophenecarboxylic acid and 2-(4-methoxyphenyl)ethanamine to give N-[2-(4-methoxyphenyl)ethyl]-5-(4-pyridinyl)-2-thiophenecarboxamide (7.6 mg) as a solid.

MS m/z 339 (M+1)⁺.

### Example 42

### N-[2-(2-chlorophenyl)ethyl]-5-(4-pyridinyl)-2-thiophenecarboxamide

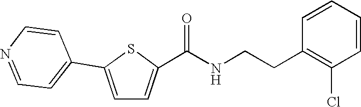

Prepared in a similar manner as described for Example 3 from 5-(4-pyridinyl)-2-thiophenecarboxylic acid and 2-(2-chlorophenyl)ethanamine to give N-[2-(2-chlorophenyl)ethyl]-5-(4-pyridinyl)-2-thiophenecarboxamide (12 mg) as a solid.

MS m/z 343 (M+1)⁺.

### Example 43

### N-[2-(3-chlorophenyl)ethyl]-5-(4-pyridinyl)-2-thiophenecarboxamide

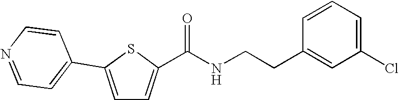

Prepared in a similar manner as described for Example 3 from 5-(4-pyridinyl)-2-thiophenecarboxylic acid and 2-(3-chlorophenyl)ethanamine to give N-[2-(3-chlorophenyl)ethyl]-5-(4-pyridinyl)-2-thiophenecarboxamide (14 mg) as a solid.

MS m/z 343 (M+1)⁺.

### Example 44

### N-[2-(4-chlorophenyl)ethyl]-5-(4-pyridinyl)-2-thiophenecarboxamide

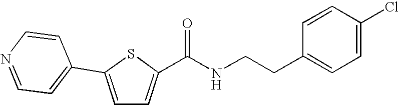

Prepared in a similar manner as described for Example 3 from 5-(4-pyridinyl)-2-thiophenecarboxylic acid and 2-(4-chlorophenyl)ethanamine to give N-[2-(4-chlorophenyl)ethyl]-5-(4-pyridinyl)-2-thiophenecarboxamide (13 mg) as a solid.

MS m/z 343 (M+1)⁺.

### Example 45

### N-[2-(2-ethoxyphenyl)ethyl]-5-(4-pyridinyl)-2-thiophenecarboxamide

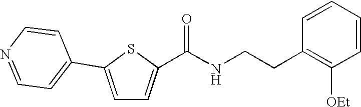

Prepared in a similar manner as described for Example 3 from 5-(4-pyridinyl)-2-thiophenecarboxylic acid and 2-(2-ethoxyphenyl)ethanamine to give N-[2-(2-ethoxyphenyl)ethyl]-5-(4-pyridinyl)-2-thiophenecarboxamide (14 mg) as a solid.

MS m/z 353 (M+1)⁺.

### Example 46

### N-[2-(3-ethoxyphenyl)ethyl]-5-(4-pyridinyl)-2-thiophenecarboxamide

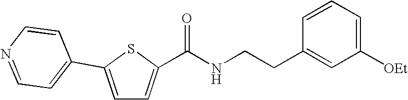

Prepared in a similar manner as described for Example 3 from 5-(4-pyridinyl)-2-O thiophenecarboxylic acid and 2-(3-ethoxyphenyl)ethanamine to give N-[2-(3-ethoxyphenyl)ethyl]-5-(4-pyridinyl)-2-thiophenecarboxamide (12 mg) as a solid.

MS m/z 353 (M+1)⁺.

### Example 47

### N-[2-(4-ethoxyphenyl)ethyl]-5-(4-pyridinyl)-2-thiophenecarboxamide

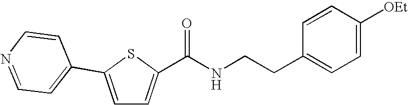

Prepared in a similar manner as described for Example 3 from 5-(4-pyridinyl)-2-thiophenecarboxylic acid and 2-(4-ethoxyphenyl)ethanamine to give N-[2-(4-ethoxyphenyl)ethyl]-5-(4-pyridinyl)-2-thiophenecarboxamide (11 mg) as a solid.

MS m/z 353 (M+1)⁺.

### Example 48

### N-[2-(2-bromophenyl)ethyl]-5-(4-pyridinyl)-2-thiophenecarboxamide

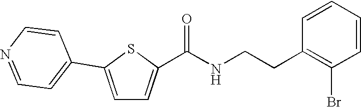

Prepared in a similar manner as described for Example 3 from 5-(4-pyridinyl)-2-thiophenecarboxylic acid and 2-(2-bromophenyl)ethanamine to give N-[2-(2-bromophenyl)ethyl]-5-(4-pyridinyl)-2-thiophenecarboxamide (13 mg) as a solid.

MS m/z 387/389 (M+1)⁺.

### Example 49

### N-[2-(3-bromophenyl)ethyl]-5-(4-pyridinyl)-2-thiophenecarboxamide

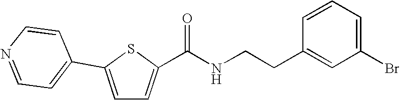

Prepared in a similar manner as described for Example 3 from 5-(4-pyridinyl)-2-thiophenecarboxylic acid and 2-(3-bromophenyl)ethanamine to give N-[2-(3-bromophenyl)ethyl]-5-(4-pyridinyl)-2-thiophenecarboxamide (12 mg) as a solid.

MS m/z 387/389 (M+1)⁺.

### Example 50

### N-[2-(4-bromophenyl)ethyl]-5-(4-pyridinyl)-2-thiophenecarboxamide

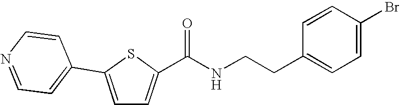

Prepared in a similar manner as described for Example 3 from 5-(4-pyridinyl)-2-thiophenecarboxylic acid and 2-(4-bromophenyl)ethanamine to give N-[2-(4-bromophenyl)ethyl]-5-(4-pyridinyl)-2-thiophenecarboxamide (13 mg) as a solid.

MS m/z 387/389 (M+1)⁺.

### Example 51

### N-[2-(2-phenoxyphenyl)ethyl]-5-(4-pyridinyl)-2-thiophenecarboxamide

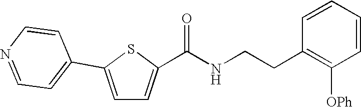

Prepared in a similar manner as described for Example 3 from 5-(4-pyridinyl)-2-thiophenecarboxylic acid and 2-(2-phenoxyphenyl)ethanamine to give N-[2-(2-phenoxyphenyl)ethyl]-5-(4-pyridinyl)-2-thiophenecarboxamide (18 mg) as a solid.

MS m/z 401 (M+1)⁺.

### Example 52

### N-[2-(4-phenoxyphenyl)ethyl]-5-(4-pyridinyl)-2-thiophenecarboxamide

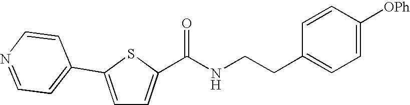

Prepared in a similar manner as described for Example 3 from 5-(4-pyridinyl)-2-thiophenecarboxylic acid and 2-(4-phenoxyphenyl)ethanamine to give N-[2-(4-phenoxyphenyl)ethyl]-5-(4-pyridinyl)-2-thiophenecarboxamide (10 mg) as a solid.

MS m/z 401 (M+1)⁺.

### Example 53

### N-[2-(4-hydroxyphenyl)ethyl]-5-(4-pyridinyl)-2-thiophenecarboxamide

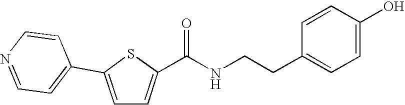

Prepared in a similar manner as described for Example 3 from 5-(4-pyridinyl)-2-thiophenecarboxylic acid and 2-(4-hydroxyphenyl)ethanamine to give N-[2-(4-hydroxyphenyl)ethyl]-5-(4-pyridinyl)-2-thiophenecarboxamide (4.7 mg) as a solid.

MS m/z 325 (M+1)⁺.

### Example 54

### N-[2-(3-trifluoromethylphenyl)ethyl]-5-(4-pyridinyl)-2-thiophenecarboxamide

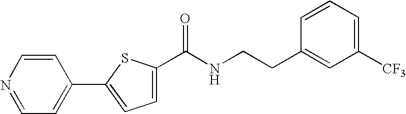

Prepared in a similar manner as described for Example 3 from 5-(4-pyridinyl)-2-thiophenecarboxylic acid and 2-(3-trifluoromethylphenyl)ethanamine to give N-[2-(3-trifluoromethylphenyl)ethyl]-5-(4-pyridinyl)-2-thiophenecarboxamide (13 mg) as a solid.

MS m/z 377 (M+1)⁺.

### Example 55

### N-{2-[4-(aminosulfonyl)phenyl]ethyl}-5-(4-pyridinyl)-2-thiophenecarboxamide

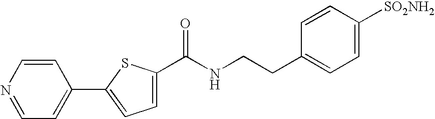

Prepared in a similar manner as described for Example 3 from 5-(4-pyridinyl)-2-thiophenecarboxylic acid and 4-(2-aminoethyl)benzenesulfonamide to give N-{2-[4-(aminosulfonyl)phenyl]ethyl}-5-(4-pyridinyl)-2-thiophenecarboxamide (12 mg) as a solid.

MS m/z 388 (M+1)⁺.

### Example 56

### 2-{[5-(4-pyridinyl)-2-thienyl]carbonyl}-1,2,3,4-tetrahydroisoquinoline

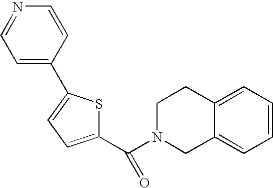

Prepared in a similar manner as described for Example 3 from 5-(4-pyridinyl)-2-thiophenecarboxylic acid and 1,2,3,4-tetrahydroisoquinoline to give 2-{[5-(4-pyridinyl)-2-thienyl]carbonyl}-1,2,3,4-tetrahydroisoquinoline (5.8 mg) as a solid.

MS m/z 321 (M+1)⁺.

### Example 57

### 5-(4-pyridinyl)-N-[2-(3-pyridinyl)ethyl]-2-thiophenecarboxamide

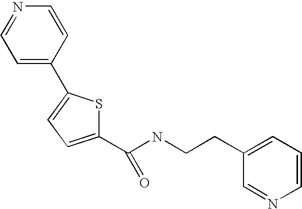

Prepared in a similar manner as described for Example 3 from 5-(4-pyridinyl)-2-thiophenecarboxylic acid and 2-(3-pyridinyl)ethanamine to give 5-(4-pyridinyl)-N-[2-(3-pyridinyl)ethyl]-2-thiophenecarboxamide (12 mg) as a solid.

MS m/z 310 (M+1)⁺.

### Example 58

### 5-(4-pyridinyl)-N-[2-(4-pyridinyl)ethyl]-2-thiophenecarboxamide

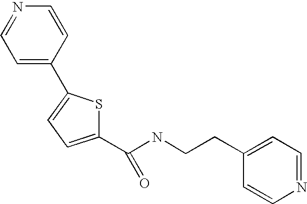

Prepared in a similar manner as described for Example 3 from 5-(4-pyridinyl)-2-thiophenecarboxylic acid and 2-(4-pyridinyl)ethanamine to give 5-(4-pyridinyl)-N-[2-(4-pyridinyl)ethyl]-2-thiophenecarboxamide (8.5 mg) as a solid.

MS m/z 310 (M+1)⁺.

### Example 59

### N-(2-phenoxyethyl)-5-(4-pyridinyl)-2-thiophenecarboxamide

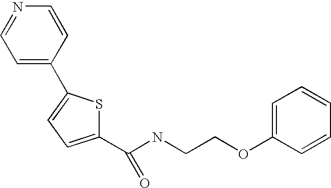

Prepared in a similar manner as described for Example 3 from 5-(4-pyridinyl)-2-thiophenecarboxylic acid and 2-phenoxyethanamine to give N-(2-phenoxyethyl)-5-(4-pyridinyl)-2-thiophenecarboxamide (12 mg) as a solid.

MS m/z 325 (M+1)⁺.

### Example 60

### N-[2-(1-piperidinyl)ethyl]-5-(4-pyridinyl-2-thiophenecarboxamide

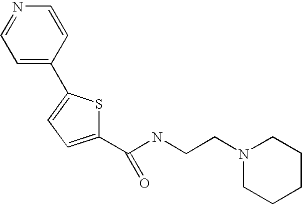

Prepared in a similar manner as described for Example 3 from 5-(4-pyridinyl)-2-thiophenecarboxylic acid and 2-(1-piperidinyl)ethanamine to give N-[2-(1-piperidinyl)ethyl]-5-(4-pyridinyl)-2-thiophenecarboxamide (7.5 mg) as a solid.

MS m/z 316 (M+1)⁺.

### Example 61

### N-[2-(4-morpholinyl)ethyl]-5-(4-pyridinyl)-2-thiophenecarboxamide

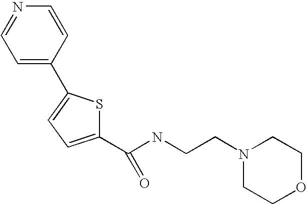

Prepared in a similar manner as described for Example 3 from 5-(4-pyridinyl)-2-thiophenecarboxylic acid and 2-(4-morpholinyl)ethanamine to give N-[2-(4-morpholinyl)ethyl]-5-(4-pyridinyl)-2-thiophenecarboxamide (7.4 mg) as a solid.

MS m/z 318 (M+1)⁺.

### Example 62

### 1-phenyl-4-{[5-(4-pyridinyl)-2-thienyl]carbonyl}piperazine

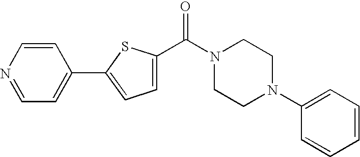

Prepared in a similar manner as described for Example 3 from 5-(4-pyridinyl)-2-thiophenecarboxylic acid and 1-phenylpiperazine to give 1-phenyl-4-{[5-(4-pyridinyl)-2-thienyl]carbonyl}piperazine (3.8 mg) as a solid.

MS m/z 350 (M+1)⁺.

### Example 63

### N-(1H-indazol-5-yl)-5-(4-pyridinyl)-2-thiophenecarboxamide

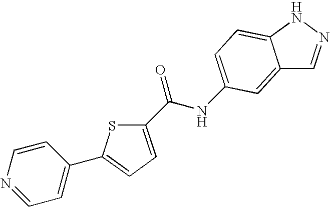

Prepared in a similar manner as described for Example 3 from 5-(4-pyridinyl)-2-thiophenecarboxylic acid and 1H-indazol-5-amine to give N-(1H-indazol-5-yl)-5-(4-pyridinyl)-2-thiophenecarboxamide (4.7 mg) as a solid.

MS m/z 321 (M+1)⁺.

### Example 64

### 1-phenyl-8-{[5-(4-pyridinyl)-2-thienyl]carbonyl}-1,3,8-triazaspiro[4.5]decan-4-one

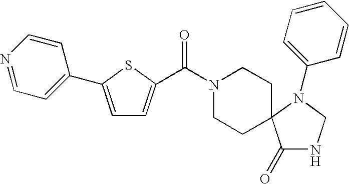

Prepared in a similar manner as described for Example 3 from 5-(4-pyridinyl)-2-thiophenecarboxylic acid and 1-phenyl-1,3,8-triazaspiro[4.5]decan-4-one to give 1-phenyl-8-{[5-(4-pyridinyl)-2-thienyl]carbonyl}-1,3,8-triazaspiro[4.5]decan-4-one (9.2 mg) as a solid.

MS m/z 419 (M+1)⁺.

### Example 65

### ethyl 4-({[5-(4-pyridinyl)-2-thienyl]carbonyl}amino)-1-piperidinecarboxylate

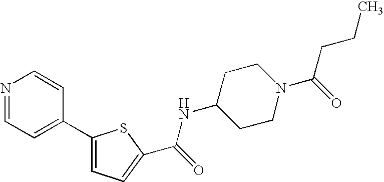

Prepared in a similar manner as described for Example 3 from 5-(4-pyridinyl)-2-thiophenecarboxylic acid and ethyl 4-amino-1-piperidinecarboxylate to give ethyl 4-({[5-(4-pyridinyl)-2-thienyl]carbonyl}amino)-1-piperidinecarboxylate (9.1 mg) as a solid.

MS m/z 360 (M+1)⁺.

### Example 66

### ethyl 1-{[5-(4-pyridinyl)-2-thienyl]carbonyl}-4-piperidinecarboxylate

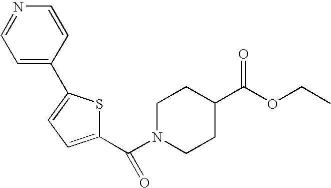

Prepared in a similar manner as described for Example 3 from 5-(4-pyridinyl)-2-thiophenecarboxylic acid and ethyl 4-piperidinecarboxylate to give ethyl 1-{[5-(4-pyridinyl)-2-thienyl]carbonyl}-4-piperidinecarboxylate (10 mg) as a solid.

MS m/z 345 (M+1)⁺.

### Example 67

### N-(1H-benzimidazol-2-ylmethyl)-5-(4-pyridinyl)-2-thiophenecarboxamide

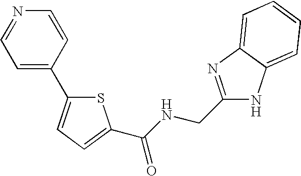

Prepared in a similar manner as described for Example 3 from 5-(4-pyridinyl)-2-thiophenecarboxylic acid and 1H-benzimidazol-2-ylmethanamine to give N-(1H-benzimidazol-2-ylmethyl)-5-(4-pyridinyl)-2-thiophenecarboxamide (3.3 mg) as a solid.

MS m/z 335 (M+1)⁺.

### Example 68

### 5-(4-pyridinyl)-N-[2-(2-pyridinyl)ethyl]-2-thiophenecarboxamide

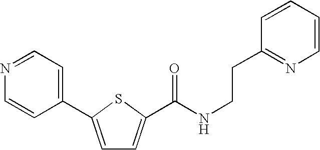

Prepared in a similar manner as described for Example 3 from 5-(4-pyridinyl)-2-thiophenecarboxylic acid and 2-(2-pyridinyl)ethanamine to give 5-(4-pyridinyl)-N-[2-(2-pyridinyl)ethyl]-2-thiophenecarboxamide (28 mg) as a solid.

MS m/z 310 (M+1)⁺.

### Example 69

### N-[2-(3-hydroxyphenyl)ethyl]-5-(4-pyridinyl)-2-thiophenecarboxamide

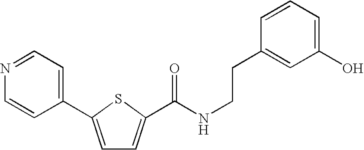

Prepared in a similar manner as described for Example 3 from 5-(4-pyridinyl)-2-thiophenecarboxylic acid and 3-(2-aminoethyl)phenol to give N-[2-(3-hydroxyphenyl)ethyl]-5-(4-pyridinyl)-2-thiophenecarboxamide (3.3 mg) as a solid.

MS m/z 325 (M+1)⁺.

### Example 70

### N-[(1R)-1-phenylethyl]-5-(4-pyridinyl)-2-thiophenecarboxamide

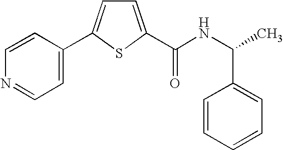

Prepared in a similar manner as described for Example 3 from 5-(4-pyridinyl)-2-thiophenecarboxylic acid and (1R)-1-phenylethanamine to give N-[(1R)-1-phenylethyl]-5-(4-pyridinyl)-2-thiophenecarboxamide (42 mg) as a solid.

MS m/z 309 (M+1)⁺.

### Example 71

### N-[(1S)-1-phenylethyl]-5-(4-pyridinyl)-2-thiophenecarboxamide

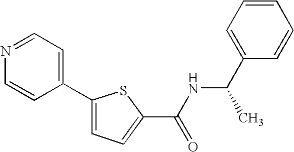

Prepared in a similar manner as described for Example 3 from 5-(4-pyridinyl)-2-thiophenecarboxylic acid and (1S)-1-phenylethanamine to give N-[(1S)-1-phenylethyl]-5-(4-pyridinyl)-2-thiophenecarboxamide (39 mg) as a solid.

MS m/z 309 (M+1)⁺.

### Example 72

### N-[(1R)-1-(3-methoxyphenyl)ethyl]-5-(4-pyridinyl)-2-thiophenecarboxamide

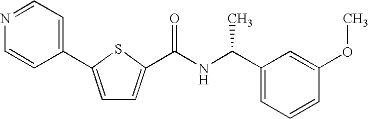

Prepared in a similar manner as described for Example 3 from 5-(4-pyridinyl)-2-thiophenecarboxylic acid and (1R)-1-(3-methoxyphenyl)ethanamine to give N-[(1R)-1-(3-methoxyphenyl)ethyl]-5-(4-pyridinyl)-2-thiophenecarboxamide (40 mg) as a solid.

MS m/z 339 (M+1)⁺.

### Example 73

### N-[(1S)-1-(3-methoxyphenyl)ethyl]-5-(4-pyridinyl)-2-thiophenecarboxamide

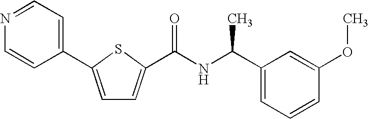

Prepared in a similar manner as described for Example 3 from 5-(4-pyridinyl)-2-thiophenecarboxylic acid and (1S)-1-(3-methoxyphenyl)ethanamine to give N-[(1S)-1-(3-methoxyphenyl)ethyl]-5-(4-pyridinyl)-2-thiophenecarboxamide (44 mg) as a solid.

MS m/z 339 (M+1)⁺.

### Example 74

### N-isopropyl-5-(4-pyridinyl)-2-thiophenecarboxamide

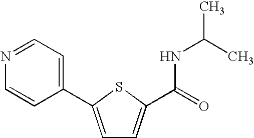

Prepared in a similar manner as described for Example 3 from 5-(4-pyridinyl)-2-thiophenecarboxylic acid and isopropylamine to give N-isopropyl-5-(4-pyridinyl)-2-thiophenecarboxamide (3.8 mg) as a solid.

MS m/z 247 (M+1)⁺.

### Example 75

### 1-methyl-4-{[5-(4-pyridinyl)-2-thienyl]carbonyl}piperazine

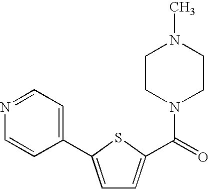

Prepared in a similar manner as described for Example 3 from 5-(4-pyridinyl)-2-thiophenecarboxylic acid and 1-methylpiperazine to give 1-methyl-4-{[5-(4-pyridinyl)-2-thienyl]carbonyl}piperazine (9.7 mg) as a solid.

MS m/z 288 (M+1)⁺.

### Example 76

### N-phenyl-5-(4-pyridinyl)-2-thiophenecarboxamide

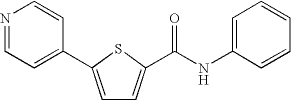

Prepared in a similar manner as described for Example 1 (Method A) from 5-bromo-2-thiophenecarbonyl chloride and aniline, followed by palladium catalyzed coupling with 4-pyridylboronic acid to give N-phenyl-5-(4-pyridinyl)-2-thiophenecarboxamide (33 mg) as a solid.

MS m/z 281 (M+1)⁺.

### Example 77

### N-(2-methoxyphenyl)-5-(4-pyridinyl)-2-thiophenecarboxamide

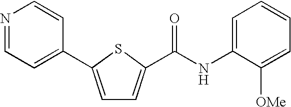

Prepared in a similar manner as described for Example 1 (Method A) from 5-bromo-2-thiophenecarbonyl chloride and o-anisidine, followed by palladium catalyzed coupling with 4-pyridylboronic acid to give N-(2-methoxyphenyl)-5-(4-pyridinyl)-2-thiophenecarboxamide (31 mg) as a solid.

MS m/z 311 (M+1)⁺.

### Example 78

### N-(2-chlorophenyl)-5-(4-pyridinyl)-2-thiophenecarboxamide

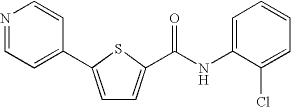

Prepared in a similar manner as described for Example 1 (Method A) from 5-bromo-2-thiophenecarbonyl chloride and 2-chloroaniline, followed by palladium catalyzed coupling with 4-pyridylboronic acid to give N-(2-chlorophenyl)-5-(4-pyridinyl)-2-thiophenecarboxamide (45 mg) as a solid.

MS m/z 315 (M+1)⁺.

### Example 79

### N-(4-methoxyphenyl)-5-(4-pyridinyl)-2-thiophenecarboxamide

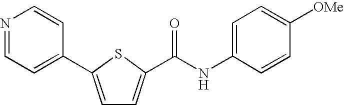

Prepared in a similar manner as described for Example 1 (Method A) from 5-bromo-2-thiophenecarbonyl chloride and p-anisidine, followed by palladium catalyzed coupling with 4-pyridylboronic acid to give N-(4-methoxyphenyl)-5-(4-pyridinyl)-2-thiophenecarboxamide (38 mg) as a solid.

MS m/z 311 (M+1)⁺.

### Example 80

### N-(4-chlorophenyl)-5-(4-pyridinyl)-2-thiophenecarboxamide

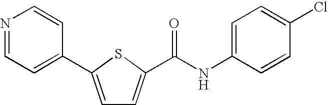

Prepared in a similar manner as described for Example 1 (Method A) from 5-bromo-2-thiophenecarbonyl chloride and 4-chloroaniline, followed by palladium catalyzed coupling with 4-pyridylboronic acid to give N-(4-chlorophenyl)-5-(4-pyridinyl)-2-thiophenecarboxamide (38 mg) as a solid.

MS m/z 315 (M+1)⁺.

### Example 81

### N-benzyl-5-(4-pyridinyl)-2-thiophenecarboxamide

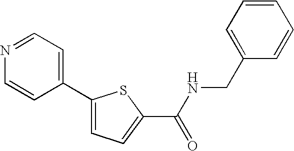

Prepared in a similar manner as described for Example 1 (Method A) from 5-bromo-2-thiophenecarbonyl chloride and benzylamine, followed by palladium catalyzed coupling with 4-pyridylboronic acid to give N-benzyl-5-(4-pyridinyl)-2-thiophenecarboxamide (40 mg) as a solid.

MS m/z 295 (M+1)⁺.

### Example 82

### Method C (see Scheme 3)

### 5-(2-amino-4-pyrimidinyl)-N-(3-methoxybenzyl)-2-thiophenecarboxamide

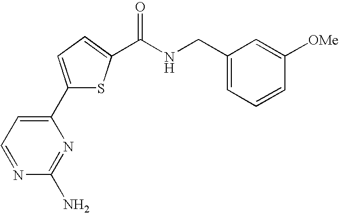

### (a) Preparation of 5-acetyl-N-(3-methoxybenzyl)-2-thiophenecarboxamide

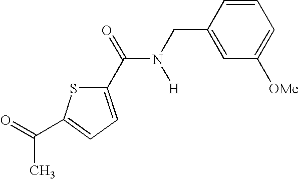

In a similar manner as described for Example 2a, 5-acetyl-2-thiophene-carboxylic acid, HOBt, EDC and 3-methoxybenzylamine were reacted to give 5-acetyl-N-(3-methoxybenzyl)-2-thiophenecarboxamide (0.593 g) as a solid.

1H NMR (400 MHz, DMSO-d6) δ ppm 2.52 (s, 3H), 3.70 (s, 3H), 4.40 (d, J=5.8 Hz, 2H), 6.79-6.86 (m, 3H), 7.22 (t, J=8.0 Hz, 1H), 7.82 (d, J=4.0 Hz, 1H), 7.90 (d, J=4.1 Hz, 1H), 9.24 (t, J=5.9 Hz, 1H); MS m/z 290 (M+1)⁺.

### (b) Preparation of 5-[(2E)-3-(dimethylamino)-2-propenoyl]-N-(3-methoxybenzyl)-2-thiophenecarboxamide

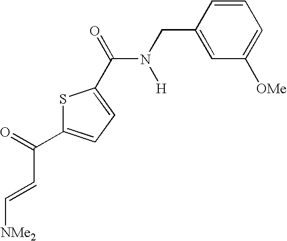

A mixture of 5-acetyl-N-(3-methoxybenzyl)-2-thiophenecarboxamide (0.59 g) and dimethylformamide dimethylacetal (3 mL) was heated at reflux for 2 h and cooled to rt. The volatiles were removed by rotary evaporation under reduced pressure and the residual solids were triturated in ether, followed by filtration of the solids to give 5-[(2E)-3-(dimethylamino)-2-propenoyl]-N-(3-methoxybenzyl)-2-thiophenecarbox-amide (0.657 g) as a brown solid.

1H NMR (400 MHz, DMSO-d6) δ ppm 2.89/3.12 (2×s, 6H), 3.70 (s, 3H), 4.38 (d, J=5.9 Hz, 2H), 5.74 (d, J=12.3 Hz, 1H), 6.78-6.86 (m, 3H), 7.21 (t, J=8.0 Hz, 1H), 7.66 (d, J=12.3 Hz, 1H), 7.72 (d, J=4.1 Hz, 1H), 7.73 (d, J=4.0 Hz, 1H), 9.06 (t, J=5.9 Hz, 1H); MS m/z 345 (M+1)⁺.

### (c) Preparation of 5-(2-amino-4-pyrimidinyl)-N-(3-methoxybenzyl)-2-thiophenecarboxamide

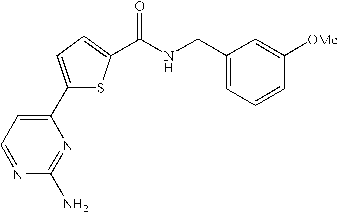

A small sphere of sodium (10 mg, 0.44 mmol) was dissolved in EtOH (4.0 mL). Guanidine hydrochloride (42 mg, 0.44 mmol) was added to the sodium ethoxide/EtOH and after 15 min. 5-[(2E)-3-(dimethylamino)-2-propenoyl]-N-(3-methoxybenzyl)-2-thiophenecarboxamide (150 mg, 0.44 mmol) was added to the reaction and heated at reflux for 3 days. The reaction was diluted with water (4 mL) and the precipitated solids were collected by filtration. The solids were rinsed with a small amount of ether and dried to give 5-(2-amino-4-pyrimidinyl)-N-(3-methoxybenzyl)-2-thiophene-carboxamide (81 mg) as a solid.

1H NMR (400 MHz, DMSO-d6) δ ppm 3.70 (s, 3H), 4.40 (d, J=5.9 Hz, 2H), 6.71 (s, 2H), 6.78-6.87 (m, 3H), 7.06 (d, J=5.1 Hz, 1H), 7.22 (t, J=8.0 Hz, 1H), 7.79 (d, J=3.9 Hz, 1H), 7.84 (d, J=4.0 Hz, 1H), 8.26 (d, J=5.1 Hz, 1H), 9.10 (t, J=6.0 Hz, 1H); MS m/z 341 (M+1)⁺.

### Example 83

### 5-(2-amino-4-pyrimidinyl)-N-benzyl-2-thiophenecarboxamide

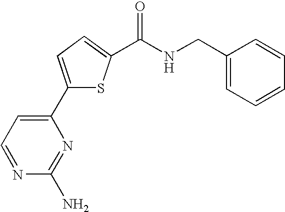

### (a) Preparation of 5-acetyl-N-benzyl-2-thiophenecarboxamide

In a similar manner as described for Example 82a, 5-acetyl-2-thiophene-carboxylic acid (5.00 g, 29.4 mmoL), HOBt, EDC and benzylamine were reacted to give 5-acetyl-N-benzyl-2-thiophenecarboxamide (6.63 g) as a solid.

1H NMR (400 MHz, DMSO-d6) δ ppm 2.59 (s, 3H), 4.50 (d, J=6.0 Hz, 2H), 7.26-7.40 (m, 5H), 7.88 (d, J=4.1 Hz, 1H), 7.96 (d, J=4.0 Hz, 1H), 9.33 (t, J=5.9 Hz, 1H); MS m/z 260 (M+1)⁺.

### (b) Preparation of 5-[(2E)-3-(dimethylamino)-2-propenoyl]-N-benzyl-2-thiophenecarboxamide

In a similar manner as described for Example 82b, 5-acetyl-N-benzyl-2-thiophenecarboxamide (2.00 g, 7.71 mmoL) and dimethylformamide dimethylacetal (10 mL) were reacted to give 5-[(2E)-3-(dimethylamino)-2-propenoyl]-N-benzyl-2-thiophenecarboxamide (2.33 g) as a solid.

1H NMR (0.400 MHz, DMSO-d6) δ ppm 2.90/3.13 (2×s, 6H), 4.43 (d, J=6.0 Hz, 2H), 5.76 (d, J=12.2 Hz, 1H), 7.23-7.34 (m, 5H), 7.68 (d, J=12.2 Hz, 1H), 7.74 (d, J=4.0 Hz, 1H), 7.75 (d, J=4.0 Hz, 1H), 9.11 (t, J=6.0 Hz, 1H); MS m/z 345 (M+1)⁺.

### (c) Preparation of 5-(2-amino-4-pyrimidinyl)-N-benzyl-2-thiophenecarboxamide

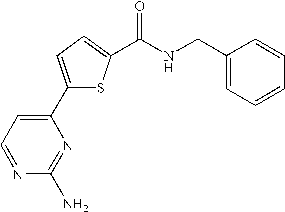

In a similar manner as described for Example 82c, sodium (16 mg) in EtOH (4.0 mL), guanidine hydrochloride (65 mg) and 5-[(2E)-3-(dimethylamino)-2-propenoyl]-N-benzyl-2-thiophenecarboxamide (0.215 g, 0.683 mmoL), were reacted to give 5-(2-amino-4-pyrimidinyl)-N-benzyl-2-thiophene-carboxamide (155 mg) as a solid.

1H NMR (400 MHz, DMSO-d6) δ ppm 4.44 (d, J=6.0 Hz, 2H), 6.73 (s, 2H), 7.08 (d, J=5.1 Hz, 1H), 7.24-7.33 (m, 5H), 7.80 (d, J=4.1 Hz, 1H), 7.86 (d, J=4.0 Hz, 1H), 8.27 (d, J=5.1 Hz, 1H), 9.14 (t, J=5.9 Hz, 1H); MS m/z 311 (M+1)⁺.

### Example 84

### 5-(4-pyrimidinyl)-N-(3-methoxybenzyl)-2-thiophenecarboxamide

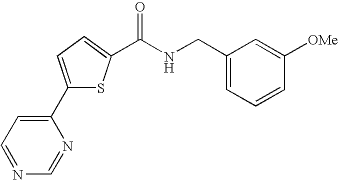

In a similar manner as described for Example 82c, K₂CO₃ (30 1 mg, 2.18 mmol), formamidine hydrochloride (105 mg, 1.31 mmol) and 5-[(2E)-3-(dimethylamino)-2-propenoyl]-N-benzyl-2-thiophenecarboxamide (150 mg, 0.436 mmoL) in EtOH (3 mL) were reacted to give after chromatography 5-(4-pyrimidinyl)-N-benzyl-2-thiophenecarboxamide (28 mg) as a solid.

1H NMR (400 MHz, DMSO-d6) δ ppm 3.71 (s, 3H), 4.41 (d, J=5.8 Hz, 2H), 6.79-6.88 (m, 3H), 7.23 (t, J=8.0 Hz, 1H), 7.86 (d, J=4.0 Hz, 1H), 8.02 (d, J=5.5 Hz, 1H), 8.05 (d, J=4.0 Hz, 1H), 8.82 (d, J=5.4 Hz, 1H), 9.12 (s, 1H), 9.17 (t, J=6.0 Hz, 1H); MS m/z 326 (M+1)⁺.

### Example 85

### Method D (Scheme 4)

### 5-(1H-indazol-5-yl)-N-(3-methoxybenzyl)-2-thiophenecarboxamide

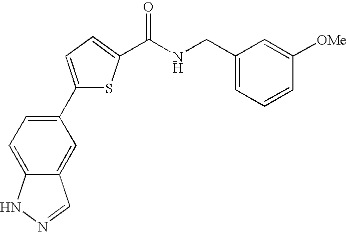

### (a) Preparation of 5-(4,4,5,5-tetramethyl-1,3,2-dioxaborolan-2-yl)-2-thiophenecarboxylic acid

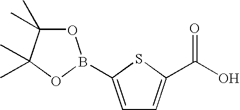

2-Carboxy-5-thiopheneboronic acid (1.04 g, 6.05 mmol) and pinacol (0.715 g, 6.05 mmol) were dissolved in a mixture of THF (15 mL) and toluene (15 mL). The volatiles were removed by rotary evaporation under reduced pressure. The solids were again treated three times with THF: toluene (10 mL:10 mL) followed by evaporation after each time to give 5-(4,4,5,5-tetramethyl-1,3,2-dioxaborolan-2-yl)-2-thiophene-carboxylic acid (1.43 g) as a white solid.

1H NMR (400 MHz, DMSO-d6) δ ppm 1.27 (s, 12H), 7.51 (d, J=3.6 Hz, 1H), 7.71 (d, J=3.7 Hz, 1H), 13.26 (brs, 1H).

### (b) Preparation of N-(3-methoxybenzyl)-5-(4,4,5,5-tetramethyl-1,3,2-dioxaborolan-2-yl)-2-thiophenecarboxamide

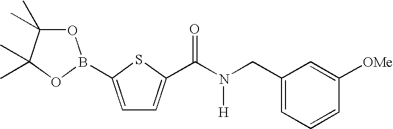

In a similar manner as described for Example 2a, 5-(4,4,5,5-tetramethyl-1,3,2-dioxaborolan-2-yl)-2-thiophenecarboxylic acid (0.700 g, 2.75 mmol), HOBt (0.446 g, 3.30 mmol), EDC (0.633 g, 3.30 mmol), 3-methoxybenzylamine (0.386 mL, 3.02 mmol) and DMF (10 mL) gave N-(3-methoxybenzyl)-5-(4,4,5,5-tetramethyl-1,3,2-dioxa-borolan-2-yl)-2-thiophenecarboxamide (0.762 g) as a yellow solid.

1H NMR (400 MHz, DMSO-d6) δ ppm 1.26 (s, 12H), 3.71 (s, 3H), 4.39 (d, J=6.0 Hz, 2H), 6.78-6.85 (m, 3H), 7.21 (t, J=8.0 Hz, 1H), 7.51 (d, J=3.7 Hz, 1H), 7.80 (d, J=3.7 Hz, 1H), 9.09 (t, J=6.0 Hz, 1H).

### (b) Preparation of 5-(1H-indazol-5-yl)-N-(3-methoxybenzyl)-2-thiophenecarboxamide

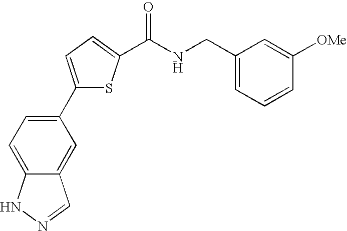

In a similar manner as described for Example 2b, N-(3-methoxybenzyl)-5-(4,4,5,5-tetramethyl-1,3,2-dioxa-borolan-2-yl)-2-thiophenecarboxamide (0.100 g, 0.27 mmol), 5-iodo-1H-indazole (0.054 g, 0.22 mmol), 1M aq. Na₂CO₃ (0.135 mL, 0-27 mmol), dichlorobis(triphenylphosphine)palladium(II) (7.7 mg, 0.011 mmol), DME (1.0 mL) and EtOH (0.5 mL) gave 5-(1H-indazol-5-yl)-N-(3-methoxybenzyl)-2-thiophenecarboxamide (10 mg) as a solid.

1H NMR (400 MHz, DMSO-d6) δ ppm 3.71 (s, 3H), 4.41 (d, J=3.9 Hz, 2H), 6.78-6.88 (m, 3H), 7.23 (t, J=8.0 Hz, 1H), 7.49 (d, J=3.7 Hz, 1H), 7.57 (d, J=8.6 Hz, 1H), 7.67 (d, J=8.6 Hz, 1H), 7.77 (d, J=3.9 Hz, 1H), 8.09 (s+d, J=9.3 Hz, 2H), 9.00 (t, J=5.9 Hz, 1H), 13.17 (s, 1H); MS m/z 364 (M+1)⁺.

### Example 86

### 5-(6-amino-4-pyrimidinyl)-N-(3-methoxybenzyl)-2-thiophenecarboxamide

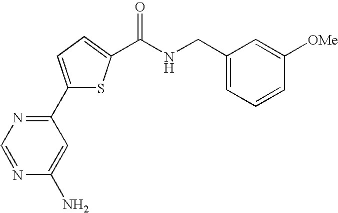

### (a) Preparation of N-allyl-6-chloro-4-pyrimidinamine

(ref.=Botta, Maurizio; Saladino, Raffaele; Pedraly-Noy, Guido; Nicoletti, Rosario synthesis of 4-[alkyl(hydroxypropyl)amino]pyrimidine and 4-[alkyl(dihydroxypropyl)amino]pyrimidine derivatives. Structural analogs of 9-(2′,3′-dihydroxypropyl)adenine [(S)-DHPA] as inhibitors of human DNA methyltransferase. Medicinal Chemistry Research (1994), 4(5), 323-34.)

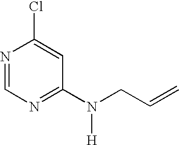

A mixture of 4,6-dichloropyrimidine (1.00 g, 6.71 mmol) and allylamine (0.528 mL, 7.05 mmol) in THF (25 mL) was stirred at rt for 20 h. The volatiles were removed by rotary evaporation under reduced pressure. The residual oil was partitioned between AcOEt: water (50 mL:10 mL) and the phases separated. The aqueous phase was extracted with AcOEt (50 mL) and the combined organic extract was dried (Na₂SO₄), filtered and concentrated to dryness to give N-allyl-6-chloro-4-pyrimidinamine (1.02 g) as a white solid.

1H NMR (400 MHz, DMSO-d6) δ ppm 3.94 (br s, 2H), 5.07 (d, J=10.2 Hz, 1H), 5.14 (d, J=17.2 Hz, 1H), 5.84 (br s, 1H), 6.51 (br s, 1H), 7.86 (br s, 1H), 8.23 (s, 1H); MS m/z 170 (M+1)⁺.

### (b) Preparation of 5-[6-(allylamino)-4-pyrimidinyl]-N-(3-methoxybenzyl)-2-thiophenecarboxamide

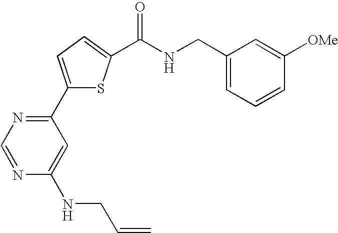

In a similar manner as described for Example 2b, N-(3-methoxybenzyl)-5-(4,4,5,5-tetramethyl-1,3,2-dioxa-borolan-2-yl)-2-thiophenecarboxamide (0.132 g, 0.35 mmol), N-allyl-6-chloro-4-pyrimidinamine (0.050 g, 0.29 mmol), 2M aq. Na₂CO₃ (0.175 mL, 0.35 mmol), dichlorobis(triphenylphosphine)palladium(II) (9.8 mg, 0.014 mmol), DME (1.0 mL) and EtOH (0.5 mL) gave 5-[6-(allylamino)-4-pyrimidinyl]-N-(3-methoxybenzyl)-2-thiophenecarboxamide (22 mg) as a solid.

1H NMR (400 MHz, DMSO-d6) δ ppm 3.70 (s, 3H), 3.95 (br s, 2H), 4.39 (d, J=5.5 Hz, 2H), 5.08 (d, J=10.3 Hz, 1H), 5.18 (d, J=17.1 Hz, 1H), 5.87 (m, 1H), 6.78-6.87 (m, 3H), 7.22 (t, J=7.8 Hz, 1H), 7.68 (s, 1H), 7.78 (d, J=3.5 Hz, 1H), 8.37 (s, 2H), 9.09 (t, J=5.9 Hz, 1H); MS m/z 381 (M+1)⁺.

### (c) Preparation of 5-(6-amino-4-pyrimidinyl)-N-(3-methoxybenzyl)-2-thiophenecarboxamide

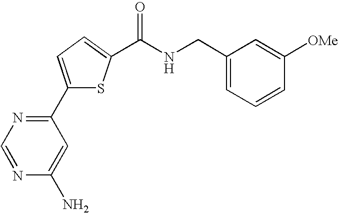

A mixture of 5-[6-(allylamino)-4-pyrimidinyl]-N-(3-methoxybenzyl)-2-thiophenecarboxamide (16 mg, 0.042 mmol), 1,3-dimethylbarbituric acid (6.6 mg, 0.042 mmol), tetrakis-(triphenylphosphine)palladium(II) (2.4 mg, 0.0021 mmol) and DCM (0.5 mL) were heated at 140° C. for 25 min in a Creator™ microwave instrument from Personal Chemistry. The volatiles were removed by rotary evaporation under reduced pressure and the residual solids were purified in a similar manner as Example 1c to give 5-(6-amino-4-pyrimidinyl)-N-(3-methoxybenzyl)-2-thiophenecarboxamide (6.8 mg) as a solid.

1H NMR (400 MHz, DMSO-d6) δ ppm 3.72 (s, 3H), 4.41 (d, J=5.8 Hz, 2H), 6.79 (s, 1H), 6.79-6.88 (m, 3H), 7.00 (br s, 2H), 7.23 (t, J=8.0 Hz, 1H), 7.70 (d, J=4.0 Hz, 1H), 7.78 (d, J=4.0 Hz, 1H), 8.33 (s, 1H), 9.11 (t, J=6.0 Hz, 1H); MS m/z 341 (M+1)⁺.

### Example 87

### N-benzyl-4-bromo-5-(4-pyridinyl)-2-thiophenecarboxamide

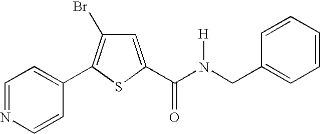

### (a) Preparation of N-benzyl-4,5-dibromo-2-thiophenecarboxamide

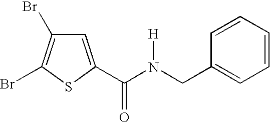

In a similar manner as Example 2, 4,5-dibromo-2-thiophenecarboxylic acid (5.00 g, 17.5 mmol), HOBt (2.84 g, 21.0 mmol), EDC (4.03 g, 21.0 mmol), benzylamine (2.10 mL, 19.2 mmol) and DMF (50 mL) gave N-benzyl-4,5-dibromo-2-thiophenecarboxamide (0.584 g) as a yellow solid.

1H NMR (400 MHz, DMSO-d6) δ ppm 4.44 (d, J=5.8 Hz, 2H), 7.26-7.36 (m, 5H), 7.84 (s, 1H), 9.20 (t, J=5.9 Hz, 1H); MS m/z 374/376/378 (M+1)⁺.

### (b) Preparation of N-benzyl-4-bromo-5-(4-pyridinyl)-2-thiophenecarboxamide

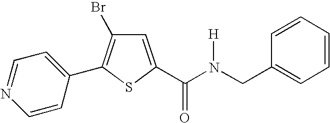

In a similar manner as Example 2, N-benzyl-4,5-dibromo-2-thiophenecarboxamide (0.200 g, 0.533 mmol), 4-pyridylboronic acid (98 mg, 0.800 mmol), 2M aq. sodium carbonate (0.400 mL, 0.800 mmol), dichlorobis(triphenylphosphine)palladium(II) (19 mg, 0.027 mmol), and DME (2.0 mL) gave N-benzyl-4-bromo-5-(4-pyridinyl)-2-thiophenecarboxamide (56 mg) as a solid.

1H NMR (400 MHz, DMSO-d6) δ ppm 4.46 (d, J=5.5 Hz, 2H), 7.25-7.35 (m, 5H), 7.69/8.70 (AB q, J=6.1 Hz, 4H), 7.98 (s, 1H), 9.27 (t, J=5.6 Hz, 1H); MS m/z 373/375 (M+1)⁺.

### Example 88

### N-benzyl-4,5-di(4-pyridinyl)-2-thiophenecarboxamide

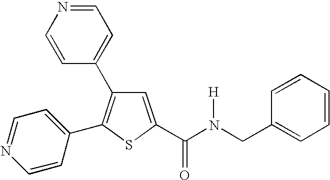

From Example 87, column chromatography gave N-benzyl-4,5-di(4-pyridinyl)-2-thiophenecarboxamide (13 mg) as a minor product of chromatography.

1H NMR (400 MHz, DMSO-d6) δ ppm 4.48 (s, 2H), 7.24-7.32 (m, 9H), 8.06 (s, 1H), 8.56 (2×d, 4H), 9.22 (br s, 1H); MS m/z 372 (M+1)⁺.

### Example 89

### N-(3-methoxybenzyl)-4,5-di(4-pyridinyl)-2-thiophenecarboxamide

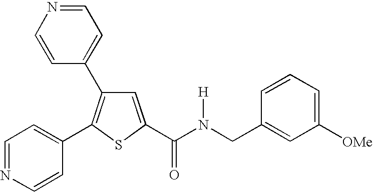

### (a) Preparation of N-(3-methoxybenzyl)-4,5-dibromo-2-thiophenecarboxamide

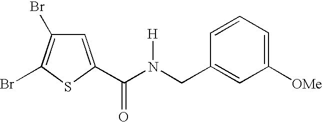

In a similar manner as Example 87a, 4,5-dibromo-2-thiophenecarboxylic acid (0.632 g, 2.49 mmol), HOBt (0.404 g, 2.99 mmol), EDC (0.573 g, 2.99 mmol), 3-methoxybenzylamine (0.351 mL, 2.74 mmol) and DMF (10 mL) gave N-(3-methoxybenzyl)-4,5-dibromo-2-thiophenecarboxamide (0.831 g) as a solid.

1H NMR (400 MHz, DMSO-d6) δ ppm 3.72 (s, 3H), 4.39 (d, J=6.0 Hz, 2H), 6.80-6.86 (m, 3H), 7.23 (t, J=8.0 Hz, 1H), 7.82 (s, 1H), 9.16 (t, J=5.8 Hz, 1H).

### (b) Preparation of N-(3-methoxybenzyl)-4,5-di(4-pyridinyl)-2-thiophenecarboxamide

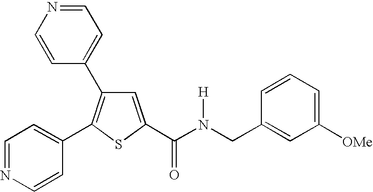

In a similar manner as Example 87b, column chromatography gave N-(3-methoxybenzyl)-4,5-di(4-pyridinyl)-2-thiophenecarboxamide (45 mg) as a solid.

1H NMR (300 MHz, DMSO-d6) δ ppm 3.78 (s, 3H), 4.49 (d, J=5.9 Hz, 2H), 6.86-6.95 (m, 3H), 7.27-7.34 (m, 5H), 8.11 (s, 1H), 8.61 (2×d, J=5.9 Hz, 4H), 9.24 (t, J=6.0 Hz, 1H); MS m/z 402 (M+1)⁺.

### Example 90

### N-benzyl-5-[2-(methylamino)-4-pyrimidinyl]-2-thiophenecarboxamide

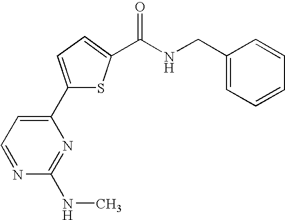

In a similar manner as described for Example 83c, sodium (10.5 mg, 0.456 mmol) in EtOH (2.0 mL), 1-methylguanidine hydrochloride (50 mg, 0.456 mmol) and 5-[(2E)-3-(dimethylamino)-2-propenoyl]-N-benzyl-2-thiophenecarboxamide (96 mg, 0.304 mmoL) gave N-benzyl-5-[2-(methylamino)-4-pyrimidinyl]-2-thiophene-carboxamide (73 mg) as a solid 1H NMR (400 MHz, DMSO-d6) δ ppm 2.81 (d, J=4.4 Hz, 3H), 4.45 (d, J=5.9 Hz, 2H), 7.08 (d, J=5.1 Hz, 1H), 7.17-7.35 (m, 6H), 7.81 (d, J=3.8 Hz, 1H), 7.87 (d, J=4.0 Hz, 1H), 8.31 (br s, 1H), 9.14 (t, J=6.0 Hz, 1H); MS m/z 325 (M+1)⁺.

### Example 91

### N-benzyl-5-(1H-pyrazol-4-yl)-2-thiophenecarboxamide

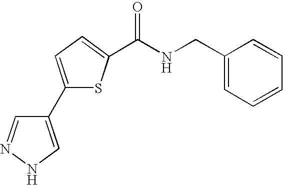

In a similar manner as Example 2b, N-benzyl-5-bromo-2-thiophenecarboxamide (148 mg, 0.50 mmol), 4-(4,4,5,5-tetramethyl-1,3,2-dioxaborolan-2-yl)-1H-pyrazole (194 mg, 1.00 mmol), potassium carbonate (138 mg, 1.00 mmol), dichlorobis(triphenylphosphine)palladium(II) (35 mg, 0.050 mmol), DMF (3.0 mL) and water (0.8 mL) gave N-benzyl-5-(1H-pyrazol-4-yl)-2-thiophene-carboxamide (45 mg) as a solid.

1H NMR (300 MHz, DMSO-d6) δ ppm 4.40 (d, 2H), 7.15-7.35 (m, 5H), 7.68 (d, 1H), 7.60 (br s, 1H), 8.14 (brs, 1H), 8.94 (t, 1H) 13.08 (brs, 1H); MS m/z 284 (M+1)⁺.

### Example 92

### N-(3-methoxybenzyl)-5-(1H-pyrazol-4-yl)-2-thiophenecarboxamide

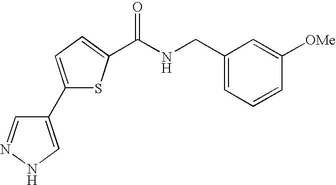

In a similar manner as Example 2b, 5-bromo-N-(3-methoxybenzyl)-2-thiophenecarboxamide (150 mg, 0.460 mmol), 4-(4,4,5,5-tetramethyl-1,3,2-dioxaborolan-2-yl)-1H-pyrazole (134 mg, 0.690 mmol), 2M aq. sodium carbonate (0.345 mL, 0.690 mmol), dichlorobis(triphenylphosphine)palladium(II) (16 mg, 0.023 mmol), DME (2.0 mL) and EtOH (1.0 mL) gave N-(3-methoxybenzyl)-5-(1H-pyrazol-4-yl)-2-thiophenecarboxamide (34 mg) as a solid.

1H NMR (300 MHz, DMSO-d6) δ ppm 3.77 (s, 3H), 4.45 (d, J=5.9 Hz, 2H), 6.84-6.93 (m, 3H), 7.25 (d, J=3.8 Hz, 1H), 7.28 (t, J=8.0 Hz, 1H), 7.75 (d, J=3.8 Hz, 1H), 7.86 (s, 1H), 8.20 (s, 1H), 8.98 (t, J=6.0 Hz, 1H) 13.14 (br s, 1H); MS m/z 314 (M+1)⁺.

### Example 93

### 5-(2-Amino-4-pyrimidinyl)-N-[[4-(methyloxy)phenyl]methyl]-2-thiophenecarboxamide (see Scheme 6)

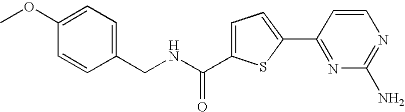

### (a) Preparation of Methyl 5-[(2E)-3-(dimethylamino)-2-propenoyl]-2-thiophenecarboxylate

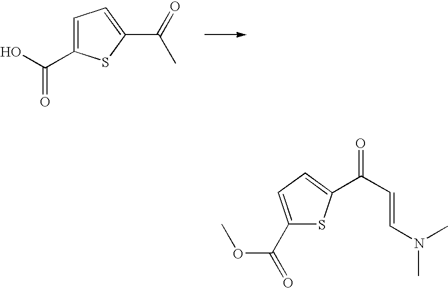

4-Acetylbenzoic acid (25 g, 147 mmol) was suspended in dimethylformamide dimethylacetal (51 mL, 3.5 eq) and the mixture was refluxed for 18 hours. The solvent was removed, and the residue was partitioned between EtOAc and water. The organic was washed with brine, and dried (MgSO₄). The solvent was evaporated to give a product (27.8 g, 79%) as a marron powder.

### (b) Preparation of 5-(2-Amino-4-pyrimidinyl)-2-thiophenecarboxylic acid

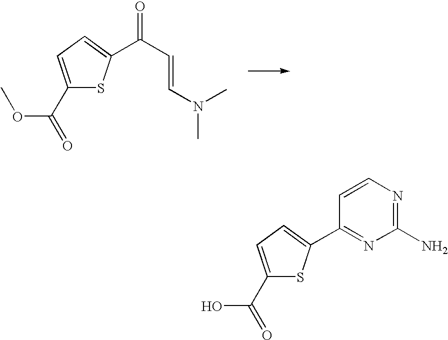

To a solution of methyl 5-[(2E)-3-(dimethylamino)-2-propenoyl]-2-thiophenecarboxylate (27.81 g, 116 mmol) in 2-methoxyethanol (650 mL) were added guanidine HCl salt (12.18 g, 127.8 mmol) and K₂CO₃ (68.1 g, 348.6 mmol). The mixture was heated at reflux for 6 hours then room temperature over night. 1 equivalent of K₂CO₃ (16 g) was added and the reaction mixture was stirred at reflux for 2 hours. A precipitate was formed and filtered. The filtrate was concentrated and washed with ether. Both of solids were combined and dissolved in methanol and acetyl chloride (5.5 eq) was added to make the solution acidic. The solution was stirred for 2 hours, and the solvent was removed to give 94.7 g of solid (contained KCl and 2-methoxyethanol). This solid was dissolved in DMF (500 mL) and stirred at reflux for 3 hours. The insoluble solid was filtered, washed with MeOH, and dissolved again in DMF (500 mL). The suspension was stirred at reflux for 2 hours. The insoluble solid was filtered, washed with MeOH. The filtrates were combined and concentrated to give a product (24.6 g, contained some DMF) as brown solid.

### (c) Preparation of 5-(2-Amino-4-pyrimidinyl)-N-{[4-(methyloxy)phenyl]methyl}-2-thiophenecarboxamide

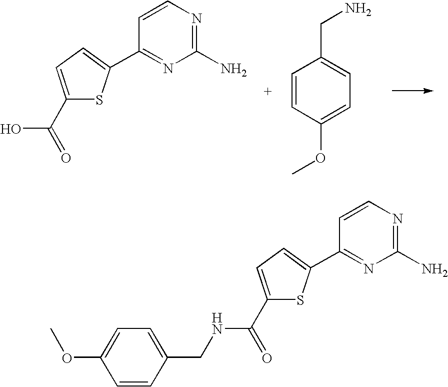

EDCI (190.6 mg, 0.99 mmol) was added to a solution of 5-(2-amino-4-pyrimidinyl)-2-thiophenecarboxylic acid (100 mg, 0.45 mmol) in DMF (3.3 mL). After 10 minutes of stirring at room temperature, HOBT (67.2 mg, 0.50 mmol) was added. The mixture was stirred at room temperature for 10 minutes, then 1-[4-(methyloxy)phenyl]methanamine (68 mg, 0.50 mmol) was added. After 30 minutes of stirring, a second portion of EDCI (95.3 mg) was added to the reaction mixture. The reaction mixture was then stirred over night at room temperature. The reaction mixture was diluted with EtOAc, washed with water, brine, dried (MgSO₄), and concentrated. The residue was purified on silica gel by flash chromatography to give 17 mg of product (11%). MS m/z 341.1 (M+1)⁺.

The following compounds were all prepared in a similar manner to Example 93 above.

### Example 94

### 5-(2-amino-4-pyrimidinyl)-N-[3-(dimethylamino)-2,2-dimethylpropyl]-2-thiophenecarboxamide

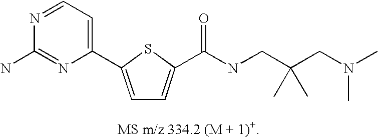

### Example 95

### 5-(2-amino-4-pyrimidinyl)-N-(2-phenylethyl)-2-thiophenecarboxamide

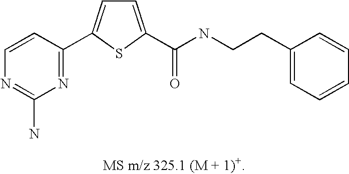

### Example 96

### 5-{2-[(4-fluorophenyl)amino]-4-pyrimidinyl}-N-(phenylmethyl)-2-thiophenecarboxamide

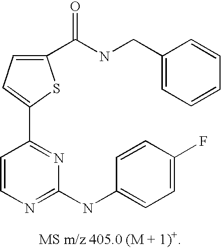

### Example 97

### 5-{2-[(4-chlorophenyl)amino]-4-pyrimidinyl}-N-(phenylmethyl)-2-thiophenecarboxamide

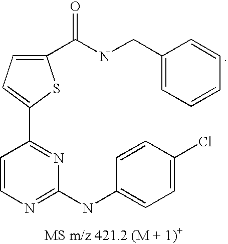

### Example 98

### 5-(2-{[4-(methyloxy)phenyl]amino}-4-pyrimidinyl)-N-(phenylmethyl)-2-thiophenecarboxamide

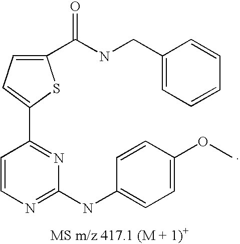

### Example 99

### 5-[2-(methylamino)-4-pyrimidinyl]-N-(phenylmethyl)-2-thiophenecarboxamide

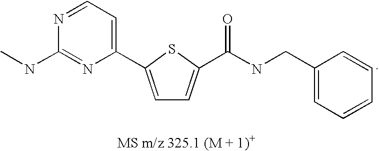

### Example 100

### N-(phenylmethyl)-5-{2-[(phenylmethyl)amino]-4-pyrimidinyl}-2-thiophenecarboxamide

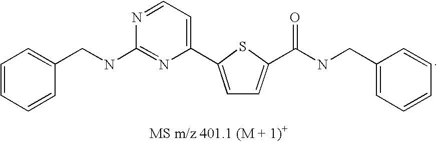

### Example 101

### 5-(2-amino-4-pyrimidinyl)-N-{[3-(methyloxy)phenyl]methyl}-2-thiophenecarboxamide

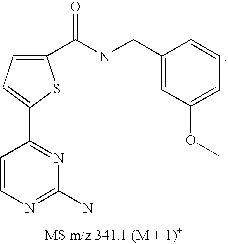

### Example 102

### 5-(2-{[4-(methyloxy)phenyl]amino}-4-pyrimidinyl)-N-{[3-(methyloxy)phenyl]methyl}-2-thiophenecarboxamide

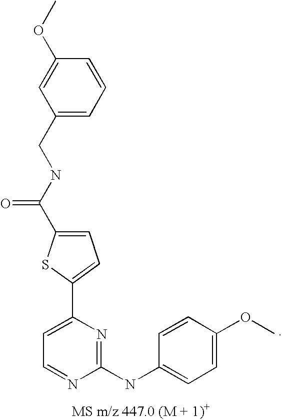

### Example 103

### N-[(2-fluorophenyl)methyl]-5-(2-{[4-(methyloxy)phenyl]amino}-4-pyrimidinyl)-2-thiophenecarboxamide

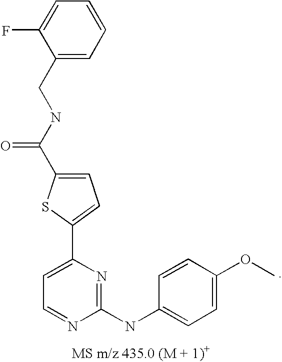

### Example 104

### N-[(3-fluorophenyl)methyl]-5-(2-{[4-(methyloxy)phenyl]amino}-4-pyrimidinyl)-2-thiophenecarboxamide

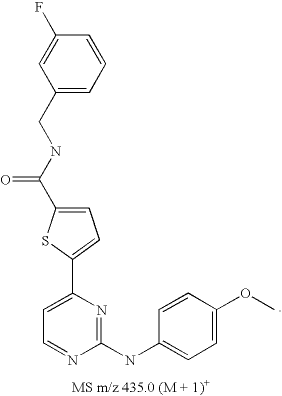

### Example 105

### N-[(4-fluorophenyl)methyl]-5-(2-{[4-(methyloxy)phenyl]amino}-4-pyrimidinyl)-2-thiophenecarboxamide

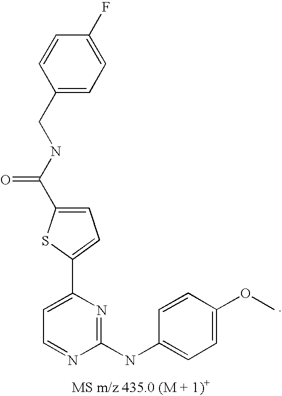

### Example 106

### N-[(2-chlorophenyl)methyl]-5-(2-{[4-(methyloxy)phenyl]amino}-4-pyrimidinyl)-2-thiophenecarboxamide

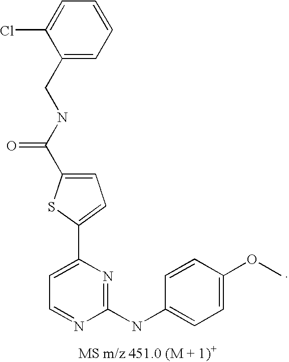

### Example 107

### N-[(3-chlorophenyl)methyl]-5-(2-{[4-(methyloxy)phenyl]amino}-4-pyrimidinyl)-2-thiophenecarboxamide

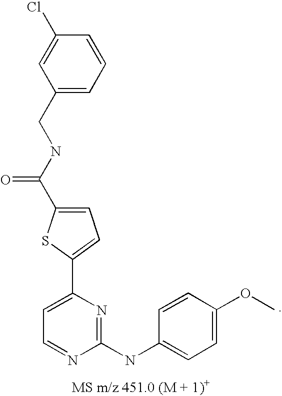

### Example 108

### N-[(4-chlorophenyl)methyl]-5-(2-{[4-(methyloxy)phenyl]amino}-4-pyrimidinyl)-2-thiophenecarboxamide

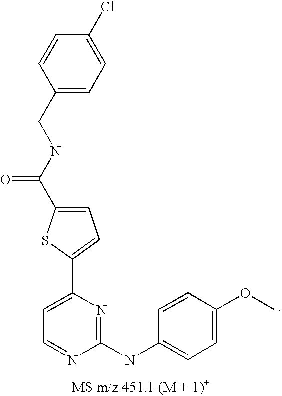

### Example 109

### 5-(2-{[4-(methyloxy)phenyl]amino}-4-pyrimidinyl)-N-{[2-(methyloxy)phenyl]methyl}-2-thiophenecarboxamide

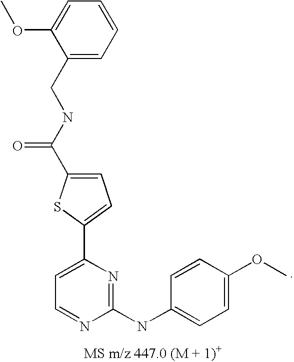

### Example 110

### 5-(2-{[4-(methyloxy)phenyl]amino}-4-pyrimidinyl)-N-{[4-(methyloxy)phenyl]methyl)-2-thiophenecarboxamide

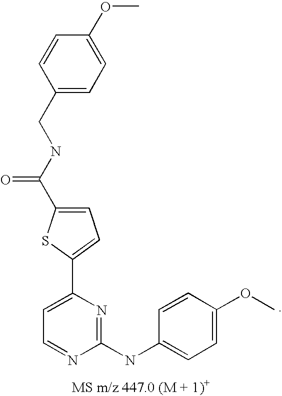

### Example 111

### N-(2,3-dihydro-1H-inden-1-yl)-5-(2-[4-(methyloxy)phenyl]amino}-4-pyrimidinyl)-2-thiophenecarboxamide

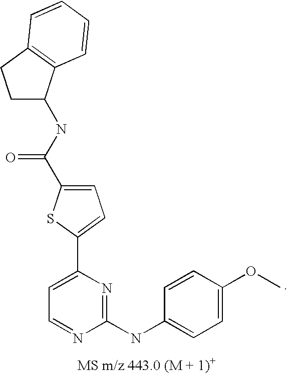

### Example 112

### 5-(2-{[4-(methyloxy)phenyl]amino}-4-pyrimidinyl)-N-(2-phenylethyl)-2-thiophenecarboxamide

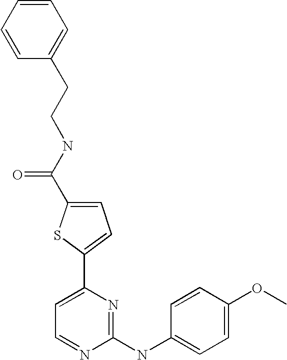

### Example 113

### 5-(2-{[4-(methyloxy)phenyl]amino}-4-pyrimidinyl)-N-[(1R)-1-phenylethyl]-2-thiophenecarboxamide

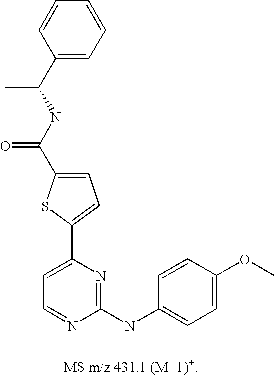

### Example 114

### 5-(2-{[4-(methyloxy)phenyl]amino}-4-pyrimidinyl)-N-[(1S)-1-phenylethyl]-2-thiophenecarboxamide

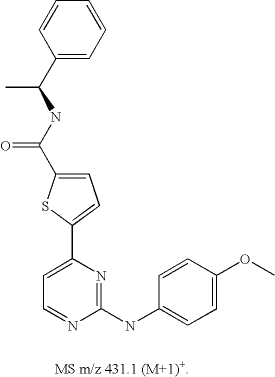

### Example 115

### 5-(2-amino-4-pyrimidinyl)-N-[(2-fluorophenyl)methyl]-2-thiophenecarboxamide

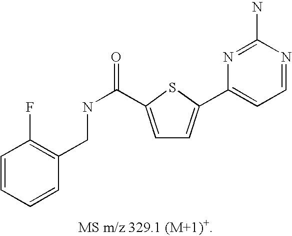

### Example 116

### 5-(2-amino-4-pyrimidinyl)-N-{[2-(methyloxy)phenyl]methyl}-2-thiophenecarboxamide

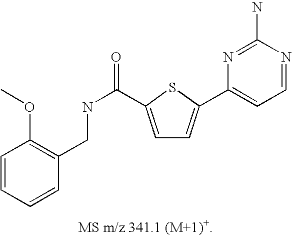

### Example 117

### 5-(2-amino-4-pyrimidinyl)-N-[(2-methyl phenyl)methyl]-2-thiophenecarboxamide

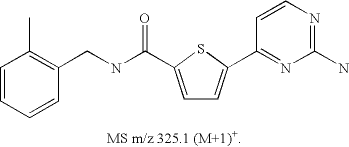

### Example 118

### 5-(2-amino-4-pyrimidinyl)-N-[(3-chlorophenyl)methyl]-2-thiophenecarboxamide

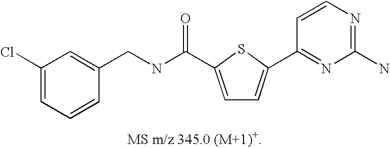

### Example 119

### 5-(2-amino-4-pyrimidinyl)-N-[(2-chlorophenyl)methyl]-2-thiophenecarboxamide

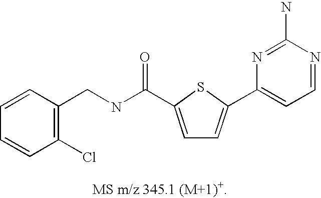

### Example 120

### 5-(2-amino-4-pyrimidinyl)-N-{(1S)-1-[3-(methyloxy)phenyl]ethyl}-2-thiophenecarboxamide

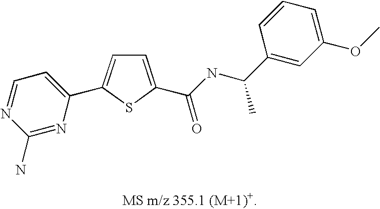

### Example 121

### 5-(2-amino-4-pyrimidinyl)-N-{(1R)-1-[3-(methyloxy)phenyl]ethyl}-2-thiophenecarboxamide

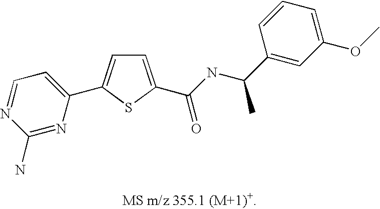

### Example 122

### 5-(2-amino-4-pyrimidinyl)-N-[(4-chlorophenyl)methyl]-2-thiophenecarboxamide

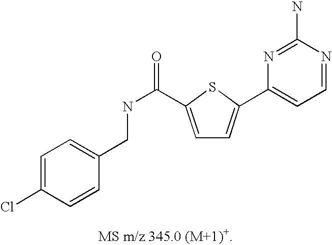

### Example 123

### 5-(2-amino-4-pyrimidinyl)-N-[(4-fluorophenyl)methyl]-2-thiophenecarboxamide

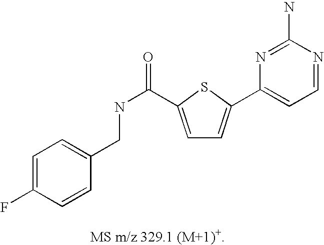

### Example 124

### 1,1-dimethylethyl ({4-[({[5-(2-amino-4-pyrimidinyl)-2-thienyl]carbonyl}amino)methyl]phenyl}methyl)carbamate

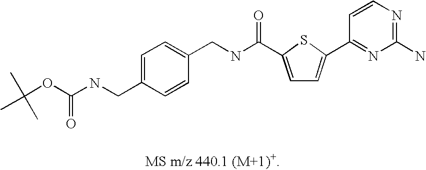

### Example 125

### 5-(2-amino-4-pyrimidinyl)-N-{1-[3-(methyloxy)phenyl]ethyl}-2-thiophenecarboxamide

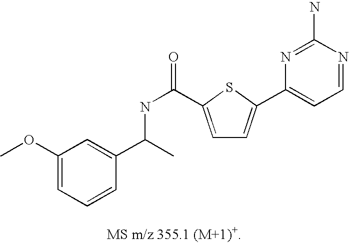

### Example 126

### 5-(2-amino-4-pyrimidinyl)-N-(1-phenylpropyl)-2-thiophenecarboxamide

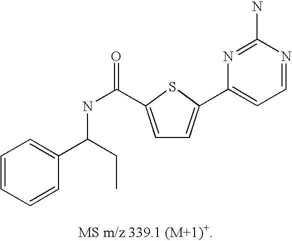

### Example 127

### 5-(2-amino-4-pyrimidinyl)-N-phenyl-2-thiophenecarboxamide

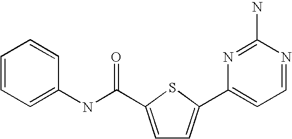

### Example 128

### 5-(2-amino-4-pyrimidinyl)-N-[(3-bromophenyl)methyl]-2-thiophenecarboxamide

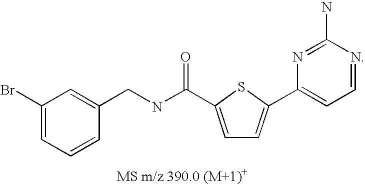

### Example 129

### 5-(2-amino-4-pyrimidinyl)-N-[(1S)-1-phenylpropyl]-2-thiophenecarboxamide

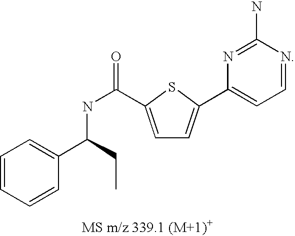

### Example 130

### 5-(2-amino-4-pyrimidinyl)-N-[(1R)-1-phenylethyl]-2-thiophenecarboxamide

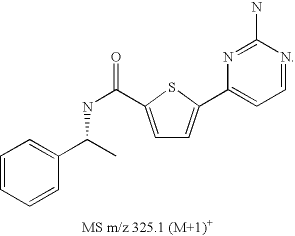

### Example 131

### 5-(2-amino-4-pyrimidinyl)-N-[(3-fluorophenyl)methyl]-2-thiophenecarboxamide

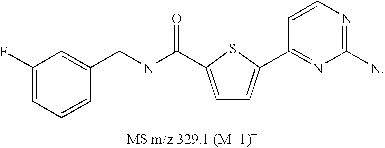

### Example 132

### 5-(2-amino-4-pyrimidinyl)-N-({3-[(difluoromethyl)oxy]phenyl}methyl)-2-thiophenecarboxamide

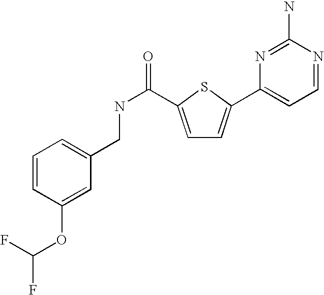

### Example 133

### 5-(2-amino-4-pyrimidinyl)-N-(1-methyl-1-phenylethyl)-2-thiophenecarboxamide

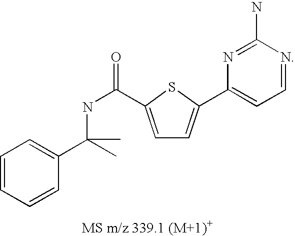

### Example 134

### N-[(3-aminophenyl)methyl]-5-(2-amino-4-pyrimidinyl)-2-thiophenecarboxamide

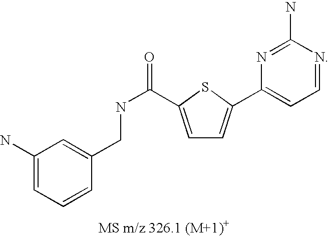

### Example 135

### 5-(2-amino-4-pyrimidinyl)-N-(2,3-dihydro-1H-inden-1-yl)-2-thiophenecarboxamide

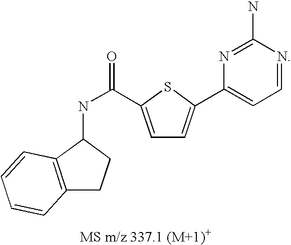

### Example 136

### 5-(2-amino-4-pyrimidinyl)-N-[(1S)-1,2,3,4-tetrahydro-1-naphthalenyl]-2-thiophenecarboxamide

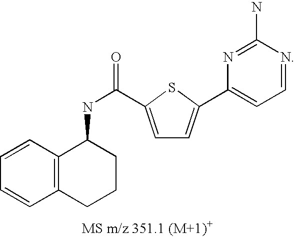

### Example 137

### 5-(2-amino-4-pyrimidinyl)-N-[(1R)-1,2,3,4-tetrahydro-1-naphthalenyl]-2-thiophenecarboxamide

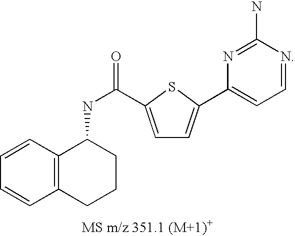

### Example 138

### 1,1-dimethylethyl {3-[1-({[5-(2-amino-4-pyrimidinyl)-2-thienyl]carbonyl}amino)ethyl]phenyl}carbamate

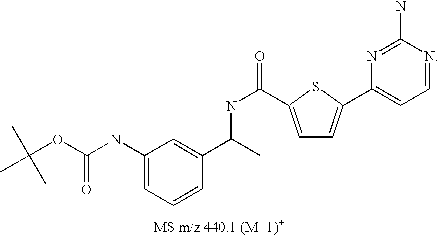

### Example 139

### 5-(2-amino-4-pyrimidinyl)-N-[(2,4-dichlorophenyl)methyl]-2-thiophenecarboxamide

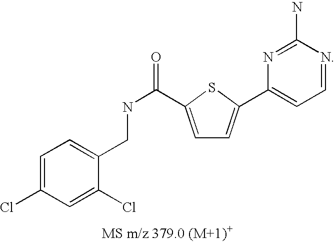

### Example 140

### 5-(2-amino-4-pyrimidinyl)-N-[(1S)-1-phenylethyl]-2-thiophenecarboxamide

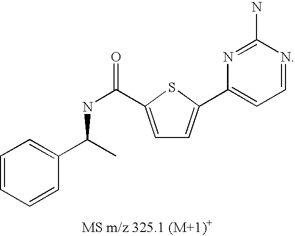

### Example 141

### 5-(2-amino-4-pyrimidinyl)-N-{[3-(trifluoromethyl)phenyl]methyl}-2-thiophenecarboxamide

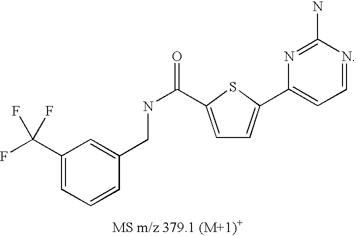

### Example 142

### 5-(2-amino-4-pyrimidinyl)-N-(3-biphenylylmethyl)-2-thiophenecarboxamide

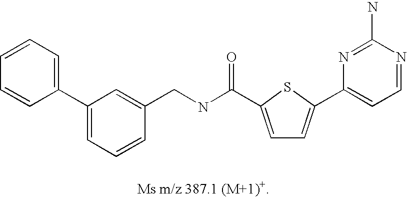

### Example 143

### 4-[5-(2,3-dihydro-1H-indol-1-ylcarbonyl)-2-thienyl]-2-pyrimidinamine

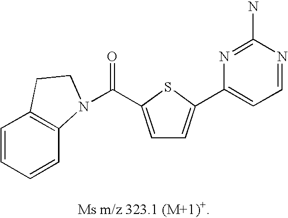

### Example 144

### 4-[5-(3,4-dihydro-1(2H)-quinolinylcarbonyl)-2-thienyl]-2-pyrimidinamine

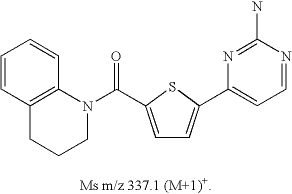

### Example 145

### 5-(2-amino-4-pyrimidinyl)-N-[(3-hydroxyphenyl)methyl]-2-thiophenecarboxamide

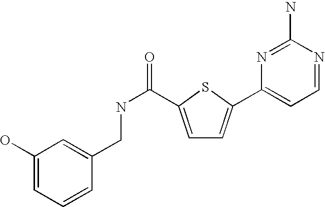

### Example 146

### 4-[5-(3,4-dihydro-2(1H)-isoquinolinylcarbonyl)-2-thienyl]-2-pyrimidinamine

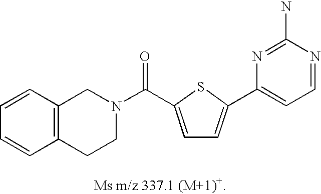

### Example 147

### 5-(2-amino-4-pyrimidinyl)-N-(1,3-benzodioxol-5-ylmethyl)-2-thiophenecarboxamide

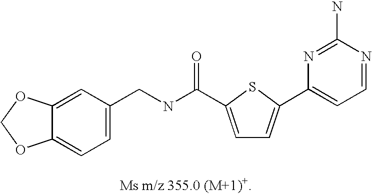

### Example 148

### 5-(2-amino-4-pyrimidinyl)-N-[(1R)-1-(3-hydroxyphenyl)ethyl]-2-thiophenecarboxamide

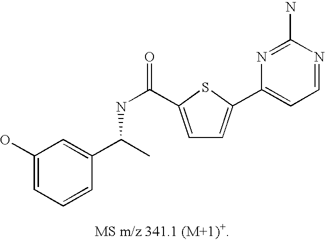

### Example 149

### 5-(2-amino-4-pyrimidinyl)-N-[(1S,2R)-2-hydroxy-2,3-dihydro-1H-inden-1-yl]-2-thiophenecarboxamide

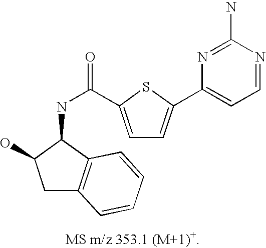

### Example 150

### 5-(2-amino-4-pyrimidinyl)-N-[(1S)-2-hydroxy-1-phenylethyl]-2-thiophenecarboxamide

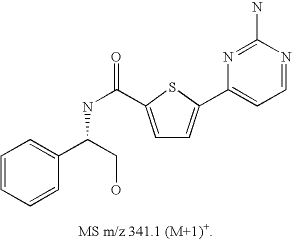

### Example 151

### 5-(2-amino-4-pyrimidinyl)-N-[(1R,2S)-2-hydroxy-2,3-dihydro-1H-inden-1-yl]-2-thiophenecarboxamide

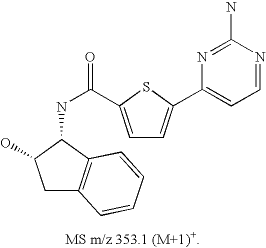

### Example 152

### 5-(2-amino-4-pyrimidinyl)-N-[1-(3,5-dichlorophenyl)-2-hydroxyethyl]-2-thiophenecarboxamide

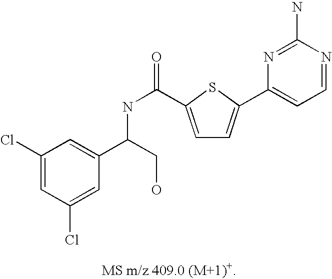

### Example 153

### 5-(2-amino-4-pyrimidinyl)-N-[(1R)-2-hydroxy-1-phenylethyl]-2-thiophenecarboxamide

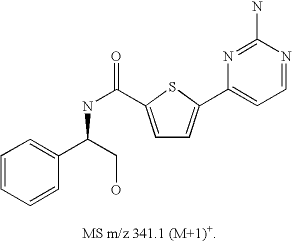

### BIOLOGICAL DATA

### Rock Kinase Assay:

ROCK inhibitor activity was determined using human recombinant ROCK1 kinase domain (amino acid 2-543) expressed in Sf9 cells (see WO9967283). The enzyme was purified using His-tag NTA column and Source 15 HPLC chromatography. The assay of Rock-1 activity involved incubation with peptide substrate and ATP³³, the subsequent incorporation of P³³ into the peptide was quantified by Scintillation Proximity Assay (SPA-Amersham Pharmacia).

For IC50 determination, test compounds were typically dissolved at 10 mM in 100% DMSO, with subsequent serial dilution in 100% DMSO. Compounds were typically assayed over an eleven point dilution range with a concentration in the assay of 50 uM to 0.8 nM, in 3-fold dilutions. IC50 values were calculated by bespoke curve fitting software and then converted to plC50.

Assays were performed in opaque, white walled, 384 well plates, in a total assay volume of 20 ul. The assays contained: 1 nM hROCK1; 1 uM biotinylated peptide (biotin-Ahx-AKRRRLSSLRA-CONH₂); 1 uM ATP; 1.85 kBq per well ATP(□-33P); 25 mM Hepes pH 7.4; mM MgCl₂; 0.015% BSA. The reactions were incubated at 22° C. for 120 minutes, then terminated by the addition of a 50 ul solution containing 60 mM EDTA and streptavidin PVT SPA beads. The SPA beads were added to a concentration of 0.14 mg per well. The plates were allowed to incubate at 22° C. for 10 minutes before centrifugation at 1500 rpm for 1 minute. P³³ incorporation was quantified by scintillation counting in a Packard TopCount.

All exemplified Examples were run with the recited assay and showed inhibitory activity versus Rock-1 with a plC₅₀ of 5.0 or greater.

1. A compound of Formula (I) or a salt or solvate thereof: 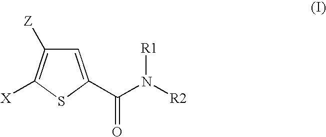
wherein:
R1 is hydrogen or C₁₋₆alkyl;
R2 is selected from the group consisting of C₁₋₆alkyl, C₁₋₄alkylNR⁷R⁸ (wherein R⁷ and R⁸ are independently H or C₁₋₄alkyl), aryl, CH(CH₂OH)aryl, arylC₁₋₆alkyl, aryloxyC₁₋₆alkyl, heteroaryl, heteroarylC₁₋₆alkyl, heterocyclyl and heterocyclylC₁₋₆alkyl, wherein in each case the aryl, heteroaryl or heterocyclyl moiety is optionally substituted by one to five groups selected from the group consisting of halogen, NH₂, hydroxy, cyano, C₁₋₄alkyl, —OCH₂O—, C₁₋₄alkoxy, haloC₁₋₄alkyl, haloC₁₋₄alkoxy, aryl, aryloxy, C₁₋₄alkoxycarbonyl, C₁₋₄hydroxyalkyl, C₁₋₄alkanoyl, C₁₋₄alkylsulfonyl, (CH₂)₀₋₄NHCOOC₁₋₄alkyl, and a group R₃R₄NSO₂ (wherein R₃ and R₄ are independently hydrogen or C₁₋₄alkyl) and a 5- or 6-membered heteroaryl group;
or R1 and R2, together with the nitrogen atom to which they are joined, form a 5- or 6-membered monocyclic heterocyclic ring or a 9- or 10-membered bicyclic heterocyclic ring wherein at least the ring which contains the nitrogen atom to which R1 and R2 are joined is non-aromatic, and wherein the 5- or 6-membered monocyclic heterocyclic ring or the 9- or 10-membered bicyclic heterocyclic ring is optionally substituted by one to four groups selected from the group consisting of halogen, hydroxy, cyano, C₁₋₄alkanoyl, oxo, C₁₋₄alkyl, C₁₋₄alkoxy, haloC₁₋₄alkyl, haloC₁₋₄alkoxy, aryl, aryloxy and C₁₋₄alkoxycarbonyl;
X is indazolyl, pyrazolyl or a group 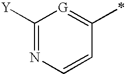
wherein
G is CH or N; and
Y is hydrogen or a group NR5R6 (wherein R5 and R6 are independently hydrogen, C₁₋₆alkyl), (CH₂)₀₋₆-phenyl (wherein the phenyl group is optionally substituted by halogen or OC₁₋₄alkyl);
and
Z is hydrogen, halogen, cyano or a 5- or 6-membered heteroaryl.

2. A compound as claimed in claim 1, wherein R1 is hydrogen.

3. A compound as claimed in claim 1, wherein R2 is arylC₁₋₆alkyl optionally substituted by one or two groups selected from the group consisting of halogen, hydroxy, C₁₋₄alkyl, C₁₋₄alkoxy, haloC₁₋₄alkyl, haloC₁₋₄alkoxy, thiadiazolyl and a group R₃R₄NSO₂ wherein R₃ and R₄ are independently hydrogen or C₁₋₄alkyl.

4. A compound as claimed in claim 1, wherein R1 and R2, together with the nitrogen atom to which they are joined, form a 6-membered monocyclic heterocyclic ring or a 10-membered bicyclic heterocyclic ring wherein at least the ring which each contains the nitrogen atom to which R1 and R2 are joined is non-aromatic, wherein the 6-membered monocyclic heterocyclic ring or 10-membered bicyclic heterocyclic ring are both optionally substituted by one or two groups selected from oxo, C₁₋₄alkyl, phenyl and C₁₋₄alkoxycarbonyl.

5. A compound as claimed in claim 1, wherein X is indazolyl or pyrazolyl.

6. A compound as claimed in claim 1, wherein X is 4-pyridinyl and Y is hydrogen.

7. A compound as claimed in claim 1, wherein X is a group: 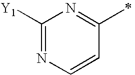
wherein Y₁ is a group NR5R6 whrein R5 and R6 are independently hydrogen or C₁₋₆alkyl.

8. A compound as claimed in claim 1 wherein Z is hydrogen or halogen.

9. A compound as claimed in claim 1, wherein said compound is selected from the group consisting of:
N-(2-phenylethyl)-5-(4-pyridinyl)-2-thiophenecarboxamide;
N-(3-methoxybenzyl)-5-(4-pyridinyl)-2-thiophenecarboxamide;
5-(4-pyridinyl)-N-(2-pyridinylmethyl)-2-thiophenecarboxamide;
N-(1-naphthylmethyl)-5-(4-pyridinyl)-2-thiophenecarboxamide;
N-(2-ethoxybenzyl)-5-(4-pyridinyl)-2-thiophenecarboxamide;
N-(2-bromobenzyl)-5-(4-pyridinyl)-2-thiophenecarboxamide;
N-(2-fluorobenzyl)-5-(4-pyridinyl)-2-thiophenecarboxamide;
N-(2-chlorobenzyl)-5-(4-pyridinyl)-2-thiophenecarboxamide;
N-(2-methylbenzyl)-5-(4-pyridinyl)-2-thiophenecarboxamide;
N-(2-trifluoromethylbenzyl)-5-(4-pyridinyl)-2-thiophenecarboxamide;
N-(2-trifluoromethoxybenzyl)-5-(4-pyridinyl)-2-thiophenecarboxamide;
N-(3-trifluoromethylbenzyl)-5-(4-pyridinyl)-2-thiophenecarboxamide;
N-(3-fluorobenzyl)-5-(4-pyridinyl)-2-thiophenecarboxamide;
N-(3-chlorobenzyl)-5-(4-pyridinyl)-2-thiophenecarboxamide;
N-(3-bromobenzyl)-5-(4-pyridinyl)-2-thiophenecarboxamide;
N-(3-iodobenzyl)-5-(4-pyridinyl)-2-thiophenecarboxamide;
N-(3-methylbenzyl)-5-(4-pyridinyl)-2-thiophenecarboxamide;
N-(3-methoxybenzyl)-5-(4-pyridinyl)-2-thiophenecarboxamide;
N-(3-trifluoromethoxybenzyl)-5-(4-pyridinyl)-2-thiophenecarboxamide;
N-(3-phenoxybenzyl)-5-(4-pyridinyl)-2-thiophenecarboxamide;
N-(4-fluorobenzyl)-5-(4-pyridinyl)-2-thiophenecarboxamide;
N-(4-bromobenzyl)-5-(4-pyridinyl)-2-thiophenecarboxamide;
N-(4-iodobenzyl)-5-(4-pyridinyl)-2-thiophenecarboxamide;
N-(4-trifluoromethylbenzyl)-5-(4-pyridinyl)-2-thiophenecarboxamide;
N-(4-methylbenzyl)-5-(4-pyridinyl)-2-thiophenecarboxamide;
N-(4-trifluoromethoxybenzyl)-5-(4-pyridinyl)-2-thiophenecarboxamide;
N-[4-(aminosulfonyl)benzyl]-5-(4-pyridinyl)-2-thiophenecarboxamide;
N-[4-(methylsulfonyl)benzyl]-5-(4-pyridinyl)-2-thiophenecarboxamide;
5-(4-pyridinyl)-N-(4-pyridinylmethyl)-2-thiophenecarboxamide;
N-benzyl-N-methyl-5-(4-pyridinyl)-2-thiophenecarboxamide;
5-(4-pyridinyl)-N-[4-(1,2,3-thiadiazol-4-yl)benzyl]-2-thiophenecarboxamide;
5-(4-pyridinyl)-N-(3-pyridinylmethyl)-2-thiophenecarboxamide;
N-[2-(2-methylphenyl)ethyl]-5-(4-pyridinyl)-2-thiophenecarboxamide;
N-[2-(3-methylphenyl)ethyl]-5-(4-pyridinyl)-2-thiophenecarboxamide;
N-[2-(4-methylphenyl)ethyl]-5-(4-pyridinyl)-2-thiophenecarboxamide;
N-[2-(2-fluorophenyl)ethyl]-5-(4-pyridinyl)-2-thiophenecarboxamide;
N-[2-(3-fluorophenyl)ethyl]-5-(4-pyridinyl)-2-thiophenecarboxamide;
N-[2-(4-fluorophenyl)ethyl]-5-(4-pyridinyl)-2-thiophenecarboxamide;
N-[2-(2-methoxyphenyl)ethyl]-5-(4-pyridinyl)-2-thiophenecarboxamide;
N-[2-(3-methoxyphenyl)ethyl]-5-(4-pyridinyl)-2-thiophenecarboxamide;
N-[2-(4-methoxyphenyl)ethyl]-5-(4-pyridinyl)-2-thiophenecarboxamide;
N-[2-(2-chlorophenyl)ethyl]-5-(4-pyridinyl)-2-thiophenecarboxamide;
N-[2-(3-chlorophenyl)ethyl]-5-(4-pyridinyl)-2-thiophenecarboxamide;
N-[2-(4-chlorophenyl)ethyl]-5-(4-pyridinyl)-2-thiophenecarboxamide;
N-[2-(2-ethoxyphenyl)ethyl]-5-(4-pyridinyl)-2-thiophenecarboxamide;
N-[2-(3-ethoxyphenyl)ethyl]-5-(4-pyridinyl)-2-thiophenecarboxamide;
N-[2-(4-ethoxyphenyl)ethyl]-5-(4-pyridinyl)-2-thiophenecarboxamide;
N-[2-(2-bromophenyl)ethyl]-5-(4-pyridinyl)-2-thiophenecarboxamide;
N-[2-(3-bromophenyl)ethyl]-5-(4-pyridinyl)-2-thiophenecarboxamide;
N-[2-(4-bromophenyl)ethyl]-5-(4-pyridinyl)-2-thiophenecarboxamide;
N-[2-(2-phenoxyphenyl)ethyl]-5-(4-pyridinyl)-2-thiophenecarboxamide;
N-[2-(4-phenoxyphenyl)ethyl]-5-(4-pyridinyl)-2-thiophenecarboxamide;
N-[2-(4-hydroxyphenyl)ethyl]-5-(4-pyridinyl)-2-thiophenecarboxamide;
N-[2-(3-trifluoromethylphenyl)ethyl]-5-(4-pyridinyl)-2-thiophenecarboxamide;
N-{2-[4-(aminosulfonyl)phenyl]ethyl}-5-(4-pyridinyl)-2-thiophenecarboxamide;
2-{[5-(4-pyridinyl)-2-thienyl]carbonyl}-1,2,3,4-tetrahydroisoquinoline;
5-(4-pyridinyl)-N-[2-(3-pyridinyl)ethyl]-2-thiophenecarboxamide;
5-(4-pyridinyl)-N-[2-(4-pyridinyl)ethyl]-2-thiophenecarboxamide;
N-(2-phenoxyethyl)-5-(4-pyridinyl)-2-thiophenecarboxamide;
N-[2-(1-piperidinyl)ethyl]-5-(4-pyridinyl)-2-thiophenecarboxamide;
N-[2-(4-morpholinyl)ethyl]-5-(4-pyridinyl)-2-thiophenecarboxamide;
1-phenyl-4-{[5-(4-pyridinyl)-2-thienyl]carbonyl}piperazine;
N-(1H-indazol-5-yl)-5-(4-pyridinyl)-2-thiophenecarboxamide;
1-phenyl-8-{[5-(4-pyridinyl)-2-thienyl]carbonyl}-1,3,8-triazaspiro[4.5]decan-4-one;
ethyl 4-({[5-(4-pyridinyl)-2-thienyl]carbonyl}amino)-1-piperidinecarboxylate;
ethyl 1-{[5-(4-pyridinyl)-2-thienyl]carbonyl}-4-piperidinecarboxylate;
N-(1H-benzimidazol-2-ylmethyl)-5-(4-pyridinyl)-2-thiophenecarboxamide;
5-(4-pyridinyl)-N-[2-(2-pyridinyl)ethyl]-2-thiophenecarboxamide;
N-[2-(3-hydroxyphenyl)ethyl]-5-(4-pyridinyl)-2-thiophenecarboxamide;
N-[(1R)-1-phenylethyl]-5-(4-pyridinyl)-2-thiophenecarboxamide;
N-[(1S)-1-phenylethyl]-5-(4-pyridinyl)-2-thiophenecarboxamide;
N-[(1R)-1-(3-methoxyphenyl)ethyl]-5-(4-pyridinyl)-2-thiophenecarboxamide;
N-[(1S)-1-(3-methoxyphenyl)ethyl]-5-(4-pyridinyl)-2-thiophenecarboxamide;
N-isopropyl-5-(4-pyridinyl)-2-thiophenecarboxamide;
1-methyl-4-{[5-(4-pyridinyl)-2-thienyl]carbonyl}piperazine;
N-phenyl-5-(4-pyridinyl)-2-thiophenecarboxamide;
N-(2-methoxyphenyl)-5-(4-pyridinyl)-2-thiophenecarboxamide;
N-(2-chlorophenyl)-5-(4-pyridinyl)-2-thiophenecarboxamide;
N-(4-methoxyphenyl)-5-(4-pyridinyl)-2-thiophenecarboxamide;
N-(4-chlorophenyl)-5-(4-pyridinyl)-2-thiophenecarboxamide;
N-benzyl-5-(4-pyridinyl)-2-thiophenecarboxamide;
5-(2-amino-4-pyrimidinyl)-N-(3-methoxybenzyl)-2-thiophenecarboxamide;
5-(2-amino-4-pyrimidinyl)-N-benzyl-2-thiophenecarboxamide;
5-(4-pyrimidinyl)-N-(3-methoxybenzyl)-2-thiophenecarboxamide;
5-(1H-indazol-5-yl)-N-(3-methoxybenzyl)-2-thiophenecarboxamide;
5-(6-amino-4-pyrimidinyl)-N-(3-methoxybenzyl)-2-thiophenecarboxamide;
N-benzyl-4-bromo-5-(4-pyridinyl)-2-thiophenecarboxamide;
N-benzyl-4,5-di(4-pyridinyl)-2-thiophenecarboxamide;
N-(3-methoxybenzyl)-4,5-di(4-pyridinyl)-2-thiophenecarboxamide;
N-benzyl-5-[2-(methylamino)-4-pyrimidinyl]-2-thiophenecarboxamide;
N-benzyl-5-(1H-pyrazol-4-yl)-2-thiophenecarboxamide; and
N-(3-methoxybenzyl)-5-(1H-pyrazol-4-yl)-2-thiophenecarboxamide;
or a salt or solvate thereof.

10-11. (canceled)

12. A method of treating a disorder in a mammal, said disorder being mediated by inappropriate ROCK-1 activity, comprising: administering to said mammal a therapeutically effective amount of a compound as defined in claim 1.

13. (canceled)

14. A pharmaceutical composition comprising a therapeutically effective amount of a compound as defined in claim 1 and one or more of pharmaceutically acceptable carriers, diluents and excipients.
